(19) United States
(12) Patent Application Publication
(10) Pub. No.: US 2004/0034192 A1
(43) Pub. Date: Feb. 19, 2004

(54) Human proteins having hyprophobic domains and dnas encoding these proteins

(76) Inventors: Seishi Kato, Sagamihara-shi (JP); Tomoko Kimura, Tsuchiura-shi (JP)

Correspondence Address: FITZPATRICK CELLA HARPER & SCINTO, 30 ROCKEFELLER PLAZA, NEW YORK, NY, 10112, US

(21) Appl. No.: 10/169,395

(22) PCT Filed: Dec. 28, 2000
(86) PCT No.: PCT/JP00/09359

(30) Foreign Application Priority Data
Jan. 6, 2000 (JP) 2000-585
Jan. 6, 2000 (JP) 2000-588
Jan. 11, 2000 (JP) 2000-2299
Feb. 3, 2000 (JP) 2000-26862
Mar. 3, 2000 (JP) 2000-58367

(51) Int. Cl. C07K 14/435; C07K 16/18; C12P 21/02; C12N 5/06; C07H 21/04

(52) U.S. Cl. 530/350; 536/23.5; 435/320.1; 435/325; 435/69.1; 530/388.1

(57) ABSTRACT

The present invention provides human proteins having hydrophobic domains, DNAs encoding these proteins, expression vectors for these DNAs, transformed eukaryotic cells expressing these DNAs and antibodies directed to these proteins.

### TECHNICAL FIELD

The present invention relates to human proteins having hydrophobic domains, DNAs encoding these proteins, expression vectors for these DNAs, eukaryotic cells expressing these DNAs and antibodies directed to these proteins. The proteins of the present invention can be employed as pharmaceuticals or as antigens for preparing antibodies directed to these proteins. The human cDNAs of the present invention can be utilized as probes for genetic diagnosis and gene sources for gene therapy. Furthermore, the cDNAs can be utilized as gene sources for producing the proteins encoded by these cDNAs in large quantities. Cells into which these genes are introduced to express secretory proteins or membrane proteins in large quantities can be utilized for detection of the corresponding receptors or ligands, screening of novel small molecule pharmaceuticals and the like. The antibodies of the present invention can be utilized for the detection, quantification, purification and the like of the proteins of the present invention.

### BACKGROUND ART

Cells secrete many proteins extracellularly. These secretory proteins play important roles in the proliferation control, the differentiation induction, the material transport, the biophylaxis, and the like of the cells. Unlike intracellular proteins, the secretory proteins exert their actions outside the cells. Therefore, they can be administered in the intracorporeal manner such as the injection or the drip, and they possess hidden potentialities as pharmaceuticals. In fact, a number of human secretory proteins such as interferons, interleukins, erythropoietin, thrombolytic agents and the like are currently employed as pharmaceuticals. In addition, secretory proteins other than those described above are undergoing clinical trials for developing their use as pharmaceuticals. It is believed that the human cells produce many unknown secretory proteins. Availability of these secretory proteins as well as genes encoding them is expected to lead to development of novel pharmaceuticals utilizing them.

On the other hand, membrane proteins play important roles, as signal receptors, ion channels, transporters and the like, in the material transport and the signal transduction through the cell membrane. Examples thereof include receptors for various cytokines, ion channels for the sodium ion, the potassium ion, the chloride ion and the like, transporters for saccharides, amino acids and the like. The genes for many of them have already been cloned. It has been clarified that abnormalities in these membrane proteins are involved in a number of previously cryptogenic diseases. Therefore, discovery of a new membrane protein is expected to lead to elucidation of the causes of many diseases, and isolation of new genes encoding the membrane proteins has been desired.

Heretofore, due to difficulty in the purification from human cells, many of these secretory proteins and membrane proteins have been isolated by genetic approaches. A general method is the so-called expression cloning method, in which a cDNA library is introduced into eukaryotic cells to express cDNAs, and the cells secreting, or expressing on the surface of membrane, the protein having the activity of interest are then screened. However, only genes for proteins with known functions can be cloned by using this method.

In general, a secretory protein or a membrane protein possesses at least one hydrophobic domain within the protein. After synthesis on ribosomes, such domain works as a secretory signal or remains in the phospholipid membrane to be entrapped in the membrane. Accordingly, if the existence of a highly hydrophobic domain is observed in the amino acid sequence of a protein encoded by a cDNA when the whole base sequence of the full-length cDNA is determined, it is considered that the cDNA encodes a secretory protein or a membrane protein.

### OBJECTS OF INVENTION

The main object of the present invention is to provide novel human proteins having hydrophobic domains, DNAs coding for these proteins, expression vectors for these DNAs, transformed eucaryotic cells that are capable of expressing these DNAs and antibodies directed to these proteins.

### SUMMARY OF INVENTION

As the result of intensive studies, the present inventors have successfully cloned cDNAs encoding proteins having hydrophobic domains from the human full-length cDNA bank, thereby completing the present invention. Thus, the present invention provides a human protein having hydrophobic domain(s), namely a protein comprising any one of amino acid sequences selected from the group consisting of SEQ ID NOS: 1 to 10, 31 to 40, 61 to 70, 91 to 100 and 121 to 130. Moreover, the present invention provides a DNA encoding said protein, exemplified by a cDNA comprising any one of base sequences selected from the group consisting of SEQ ID NOS: 11 to 30, 41 to 60, 71 to 90, 101 to 120 and 131 to 150, an expression vector that is capable of expressing said DNA by in vitro translation or in eukaryotic cells, a transformed eukaryotic cell that is capable of expressing said DNA and of producing said protein, and an antibody directed to said protein.

This object as well as other objects and advantages of the present invention will become apparent to those skilled in the art from the following description with reference to the accompanying drawings.

### BRIEF DESCRIPTION OF DRAWINGS

FIG. 1: A figure depicting the hydrophobicity/hydrophilicity profile of the protein encoded by clone HP03613.

FIG. 2: A figure depicting the hydrophobicity/hydrophilicity profile of the protein encoded by clone HP03700.

FIG. 3: A figure depicting the hydrophobicity/hydrophilicity profile of the protein encoded by clone HP03935.

FIG. 4: A figure depicting the hydrophobicity/hydrophilicity profile of the protein encoded by clone HP10755.

FIG. 5: A figure depicting the hydrophobicity/hydrophilicity profile of the protein encoded by clone HP10760.

FIG. 6: A figure depicting the hydrophobicity/hydrophilicity profile of the protein encoded by clone HP10764.

FIG. 7: A figure depicting the hydrophobicity/hydrophilicity profile of the protein encoded by clone HP10768.

FIG. 8: A figure depicting the hydrophobicity/hydrophilicity profile of the protein encoded by clone HP10769.

FIG. 9: A figure depicting the hydrophobicity/hydrophilicity profile of the protein encoded by clone HP10784.

FIG. 10: A figure depicting the hydrophobicity/hydrophilicity profile of the protein encoded by clone HP10786.

FIG. 11: A figure depicting the hydrophobicity/hydrophilicity profile of the protein encoded by clone HP03727.

FIG. 12: A figure depicting the hydrophobicity/hydrophilicity profile of the protein encoded by clone HP03801.

FIG. 13: A figure depicting the hydrophobicity/hydrophilicity profile of the protein encoded by clone HP03883.

FIG. 14: A figure depicting the hydrophobicity/hydrophilicity profile of the protein encoded by clone HP03913.

FIG. 15: A figure depicting the hydrophobicity/hydrophilicity profile of the protein encoded by clone HP10753.

FIG. 16: A figure depicting the hydrophobicity/hydrophilicity profile of the protein encoded by clone HP10758.

FIG. 17: A figure depicting the hydrophobicity/hydrophilicity profile of the protein encoded by clone HP10771.

FIG. 18: A figure depicting the hydrophobicity/hydrophilicity profile of the protein encoded by clone HP10778.

FIG. 19: A figure depicting the hydrophobicity/hydrophilicity profile of the protein encoded by clone HP10781.

FIG. 20: A figure depicting the hydrophobicity/hydrophilicity profile of the protein encoded by clone HP10785.

FIG. 21: A figure depicting the hydrophobicity/hydrophilicity profile of the protein encoded by clone HP03878.

FIG. 22: A figure depicting the hydrophobicity/hydrophilicity profile of the protein encoded by clone HP03884.

FIG. 23: A figure depicting the hydrophobicity/hydrophilicity profile of the protein encoded by clone HP03934.

FIG. 24: A figure depicting the hydrophobicity/hydrophilicity profile of the protein encoded by clone HP03949.

FIG. 25: A figure depicting the hydrophobicity/hydrophilicity profile of the protein encoded by clone HP03959.

FIG. 26: A figure depicting the hydrophobicity/hydrophilicity profile of the protein encoded by clone HP03983.

FIG. 27: A figure depicting the hydrophobicity/hydrophilicity profile of the protein encoded by clone HP10745.

FIG. 28: A figure depicting the hydrophobicity/hydrophilicity profile of the protein encoded by clone HP10775.

FIG. 29: A figure depicting the hydrophobicity/hydrophilicity profile of the protein encoded by clone HP10782.

FIG. 30: A figure depicting the hydrophobicity/hydrophilicity profile of the protein.

FIG. 31: A figure depicting the hydrophobicity/hydrophilicity profile of the protein encoded by clone HP03977.

FIG. 32: A figure depicting the hydrophobicity/hydrophilicity profile of the protein encoded by clone HP10649.

FIG. 33: A figure depicting the hydrophobicity/hydrophilicity profile of the protein encoded by clone HP10779.

FIG. 34: A figure depicting the hydrophobicity/hydrophilicity profile of the protein encoded by clone HP10790.

FIG. 35: A figure depicting the hydrophobicity/hydrophilicity profile of the protein encoded by clone HP10793.

FIG. 36: A figure depicting the hydrophobicity/hydrophilicity profile of the protein encoded by clone HP10794.

FIG. 37: A figure depicting the hydrophobicity/hydrophilicity profile of the protein encoded by clone HP10797.

FIG. 38: A figure depicting the hydrophobicity/hydrophilicity profile of the protein encoded by clone HP10798.

FIG. 39: A figure depicting the hydrophobicity/hydrophilicity profile of the protein encoded by clone HP10800.

FIG. 40: A figure depicting the hydrophobicity/hydrophilicity profile of the protein encoded by clone HP10801.

FIG. 41: A figure depicting the hydrophobicity/hydrophilicity profile of the protein encoded by clone HP03596.

FIG. 42: A figure depicting the hydrophobicity/hydrophilicity profile of the protein encoded by clone HP03882.

FIG. 43: A figure depicting the hydrophobicity/hydrophilicity profile of the protein encoded by clone HP03903.

FIG. 44: A figure depicting the hydrophobicity/hydrophilicity profile of the protein encoded by clone HP03974.

FIG. 45: A figure depicting the hydrophobicity/hydrophilicity profile of the protein encoded by clone HP03978.

FIG. 46: A figure depicting the hydrophobicity/hydrophilicity profile of the protein encoded by clone HP10735.

FIG. 47: A figure depicting the hydrophobicity/hydrophilicity profile of the protein encoded by clone HP10750.

FIG. 48: A figure depicting the hydrophobicity/hydrophilicity profile of the protein encoded by clone HP10777.

FIG. 49: A figure depicting the hydrophobicity/hydrophilicity profile of the protein encoded by clone HP10780.

FIG. 50: A figure depicting the hydrophobicity/hydrophilicity profile of the protein encoded by clone HP10795.

### DETAILED DESCRIPTION OF THE INVENTION

The proteins of the present invention can be obtained, for example, by a method for isolating proteins from human organs, cell lines or the like, a method for preparing peptides by the chemical synthesis based on the amino acid sequence of the present invention, or a method for producing proteins by the recombinant DNA technology using the DNAs encoding the hydrophobic domains of the present invention. Among these, the method for producing proteins by the recombinant DNA technology is preferably employed. For example, the proteins can be expressed in vitro by preparing an RNA by in vitro transcription from a vector having the cDNA of the present invention, and then carrying out in vitro translation using this RNA as a template. Alternatively, incorporation of the translated region into a suitable expression vector by the method known in the art may lead to expression of the encoded protein in large quantities in prokaryotic cells such as Escherichia coli and Bacillus subtilis, or eukaryotic cells such as yeasts, insect cells and mammalian cells.

In the case where the protein of the present invention is produced by expressing the DNA by in vitro translation, the protein of the present invention can be produced in vitro by incorporating the translated region of this cDNA into a vector having an RNA polymerase promoter, and then adding the vector to an in vitro translation system such as a rabbit reticulocyte lysate or a wheat germ extract, which contains an RNA polymerase corresponding to the promoter. The RNA polymerase promoters are exemplified by T7, T3, SP6 and the like. The vectors containing promoters for these RNA polymerases are exemplified by pKA1, pCDM8, pT3/T7 18, pT7/3 19, pBluescript II and the like. Furthermore, the protein of the present invention can be expressed in the secreted form or the form incorporated in the microsome membrane when a canine pancreas microsome or the like is added to the reaction system.

In the case where the protein of the present invention is produced by expressing the DNA in a microorganism such as Escherichia coli, a recombinant expression vector in which the translated region of the cDNA of the present invention is incorporated into an expression vector having an origin which is capable of replicating in the microorganism, a promoter, a ribosome-binding site, a cDNA-cloning site, a terminator and the like is constructed. After transformation of the host cells with this expression vector, the resulting transformant is cultured. Thus, the protein encoded by the cDNA can be produced in large quantities in the microorganism. In this case, a protein fragment containing any translated region can be obtained by adding an initiation codon and a termination codon in front of and behind the selected translated region and expressing the protein. Alternatively, the protein can be expressed as a fusion protein with another protein. Only the portion of the protein encoded by the cDNA can be obtained by cleaving this fusion protein with a suitable protease. The expression vectors for Escherichia coli are exemplified by the pUC series, pBluescript II, the pET expression system, the pGEX expression system and the like.

In the case where the protein of the present invention is produced by expressing the DNA in eukaryotic cells, the protein of the present invention can be produced as a secretory protein, or as a membrane protein on the surface of cell membrane, by incorporating the translated region of the cDNA into an expression vector for eukaryotic cells that has a promoter, a splicing region, a poly(A) addition site and the like, and then introducing the vector into the eukaryotic cells. The expression vectors are exemplified by pKA1, pED6dpc2, pCDM8, pSVK3, pMSG, pSVL, pBK-CMV, pBK-RSV, EBV vectors, pRS, pYES2 and the like. Examples of eukaryotic cells to be used in general include mammalian cultured cells such as monkey kidney COS7 cells and Chinese hamster ovary CHO cells, budding yeasts, fission yeasts, silkworm cells, and Xenopus oocytes. Any eukaryotic cells may be used as long as they are capable of expressing the proteins of the present invention. The expression vector can be introduced into the eukaryotic cells by using a method known in the art such as the electroporation method, the calcium phosphate method, the liposome method and the DEAE-dextran method.

After the protein of the present invention is expressed in prokaryotic cells or eukaryotic cells, the protein of interest can be isolated and purified from the culture by a combination of separation procedures known in the art. Examples of the separation procedures include treatment with a denaturing agent such as urea or a detergent, sonication, enzymatic digestion, salting-out or solvent precipitation, dialysis, centrifugation, ultrafiltration, gel filtration, SDS-PAGE, isoelectric focusing, ion-exchange chromatography, hydrophobic chromatography, affinity chromatography and reverse phase chromatography.

The proteins of the present invention also include peptide fragments (of 5 amino acid residues or more) containing any partial amino acid sequences in the amino acid sequences represented by SEQ ID NOS: 1 to 10, 31 to 40, 61 to 70, 91 to 100 and 121 to 130. These peptide fragments can be utilized as antigens for preparation of antibodies. Among the proteins of the present invention, those having the signal sequences are secreted in the form of mature proteins after the signal sequences are removed. Therefore, these mature proteins shall come within the scope of the protein of the present invention. The N-terminal amino acid sequences of the mature proteins can be easily determined by using the method for the determination of cleavage site of a signal sequence [JP-A 8-187100]. Furthermore, some membrane proteins undergo the processing on the cell surface to be converted to the secreted forms. Such proteins or peptides in the secreted forms shall also come within the scope of the protein of the present invention. In the case where sugar chain-binding sites are present in the amino acid sequences of the proteins, expression of the proteins in appropriate eukaryotic cells affords the proteins to which sugar chains are added. Accordingly, such proteins or peptides to which sugar chains are added shall also come within the scope of the protein of the present invention.

The DNAs of the present invention include all the DNAs encoding the above-mentioned proteins. These DNAs can be obtained by using a method for chemical synthesis, a method for cDNA cloning and the like.

The cDNAs of the present invention can be cloned, for example, from cDNA libraries derived from the human cells. The cDNAs are synthesized by using poly(A)⁺ RNAs extracted from human cells as templates. The human cells may be cells delivered from the human body, for example, by the operation or may be the cultured cells. The cDNAs can be synthesized by using any method such as the Okayama-Berg method [Okayama, H. and Berg, P., Mol. Cell. Biol. 2: 161-170 (1982)], the Gubler-Hoffman method [Gubler, U. and Hoffman, J., Gene 25: 263-269 (1983)] and the like. However, it is desirable to use the capping method [Kato, S. et al., Gene 150: 243-250 (1994)], as exemplified in Examples, in order to obtain a full-length clone in an effective manner. In addition, commercially available human cDNA libraries can be utilized. The cDNAs of the present invention can be cloned from the cDNA libraries by synthesizing an oligonucleotide on the basis of base sequences of any portion in the cDNA of the present invention and screening the cDNA libraries using this oligonucleotide as a probe for colony or plaque hybridization according to a method known in the art. In addition, the cDNA fragments of the present invention can be prepared from an mRNA isolated from human cells by the RT-PCR method in which oligonucleotides which hybridize with both termini of the cDNA fragment of interest are synthesized, which are then used as the primers.

The cDNAs of the present invention are characterized in that they comprise any one of the base sequences represented by SEQ ID NOS: 11 to 20, 41 to 50, 71 to 80, 101 to 110 and 131 to 140 or the base sequences represented by SEQ ID NOS: 21 to 30, 51 to 60, 81 to 90, 111 to 120 and 141 to 150. Table 1 summarizes the clone number (HP number), the cells from which the cDNA clone was obtained, the total number of bases of the cDNA, and the number of the amino acid residues of the encoded protein, for each of the cDNAs.

TABLE 1

| | | | | | Number | Number of |
|---|---|---|---|---|---|---|
| | | | | | of | amino |
| Sequence No. | | | HP No. | Cell | bases | acids |
| 1, | 11, | 21 | HP03613 | Kidney | 2865 | 578 |
| 2, | 12, | 22 | HP03700 | Kidney | 3323 | 243 |
| 3, | 13, | 23 | HP03935 | Kidney | 1585 | 461 |
| 4, | 14, | 24 | HP10755 | Kidney | 2122 | 647 |
| 5, | 15, | 25 | HP10760 | Kidney | 1775 | 446 |
| 6, | 16, | 26 | HP10764 | Kidney | 1372 | 197 |
| 7, | 17, | 27 | HP10768 | Kidney | 2074 | 540 |
| 8, | 18, | 28 | HP10769 | Kidney | 2252 | 442 |
| 9, | 19, | 29 | HP10784 | Kidney | 1461 | 262 |
| 10, | 20, | 30 | HP10786 | Kidney | 1122 | 152 |
| 31, | 41, | 51 | HP03727 | Kidney | 1617 | 335 |
| 32, | 42, | 52 | HP03801 | Umbilical cord | 1749 | 208 |
| | | | | blood | | |
| 33, | 43, | 53 | HP03883 | Kidney | 1402 | 406 |
| 34, | 44, | 54 | HP03913 | Kidney | 2474 | 618 |
| 35, | 45, | 55 | HP10753 | Umbilical cord | 3296 | 208 |
| | | | | blood | | |
| 36, | 46, | 56 | HP10758 | Kidney | 1818 | 502 |
| 37, | 47, | 57 | HP10771 | Kidney | 1646 | 336 |
| 38, | 48, | 58 | HP10778 | Kidney | 1416 | 340 |
| 39, | 49, | 59 | HP10781 | Kidney | 1927 | 223 |
| 40, | 50, | 60 | HP10785 | Kidney | 1419 | 309 |
| 61, | 71, | 81 | HP03878 | Kidney | 2016 | 599 |
| 62, | 72, | 82 | HP03884 | Kidney | 1446 | 81 |
| 63, | 73, | 83 | HP03934 | Kidney | 2467 | 654 |
| 64, | 74, | 84 | HP03949 | Kidney | 1450 | 390 |
| 65, | 75, | 85 | HP03959 | Kidney | 1897 | 452 |
| 66, | 76, | 86 | HP03983 | Kidney | 1856 | 490 |
| 67, | 77, | 87 | HP10745 | Umbilical cord | 2173 | 392 |
| | | | | blood | | |
| 68, | 78, | 88 | HP10775 | Kidney | 1934 | 538 |
| 69, | 79, | 89 | HP10782 | Kidney | 1880 | 102 |
| 70, | 80, | 90 | HP10787 | Kidney | 2295 | 442 |
| 91, | 101, | 111 | HP03977 | Kidney | 1894 | 227 |
| 92, | 102, | 112 | HP10649 | KB | 2413 | 352 |
| 93, | 103, | 113 | HP10779 | Kidney | 2376 | 130 |
| 94, | 104, | 114 | HP10790 | Kidney | 1155 | 330 |
| 95, | 105, | 115 | HP10793 | Kidney | 1329 | 350 |
| 96, | 106, | 116 | HP10794 | Kidney | 1387 | 113 |
| 97, | 107, | 117 | HP10797 | Kidney | 1158 | 189 |
| 98, | 108, | 118 | HP10798 | Kidney | 1106 | 277 |
| 99, | 109, | 119 | HP10800 | Kidney | 1907 | 274 |
| 100, | 110, | 120 | HP10801 | Kidney | 1816 | 390 |
| 121, | 131, | 141 | HP03696 | Umbilical cord | 1961 | 395 |
| | | | | blood | | |
| 122, | 132, | 142 | HP03882 | Kidney | 2194 | 550 |
| 123, | 133, | 143 | HP03903 | Kidney | 2753 | 218 |
| 124, | 134, | 144 | HP03974 | Kidney | 2085 | 596 |
| 125, | 135, | 145 | HP03978 | Kidney | 2208 | 467 |
| 126, | 136, | 146 | HP10735 | Umbilical cord | 2044 | 476 |
| | | | | blood | | |
| 127, | 137, | 147 | HP10750 | Umbilical cord | 2176 | 449 |
| | | | | blood | | |
| 128, | 138, | 148 | HP10777 | Kidney | 1363 | 105 |
| 129, | 139, | 149 | HP10780 | Kidney | 1043 | 81 |
| 130, | 140, | 150 | HP10795 | Kidney | 2435 | 552 |

The same clones as the cDNAs of the present invention can be easily obtained by screening the cDNA libraries constructed from the human cell lines or human tissues utilized in the present invention using an oligonucleotide probe synthesized on the basis of the base sequence of the cDNA provided in any one of SEQ ID NOS: 11 to 30, 41 to 60, 71 to 90, 101 to 120 and 131 to 150.

In general, the polymorphism due to the individual differences is frequently observed in human genes. Accordingly, any cDNA in which one or plural nucleotides are added, deleted and/or substituted with other nucleotides in SEQ ID NOS: 11 to 30, 41 to 60, 71 to 90, 101 to 120 and 131 to 150 shall come within the scope of the present invention.

Similarly, any protein in which one or plural amino acids are added, deleted and/or substituted with other amino acids resulting from the above-mentioned changes shall come within the scope of the present invention, as long as the protein possesses the activity of the protein having any one of the amino acid sequences represented by SEQ ID NOS: 1 to 10, 31 to 40, 61 to 70, 91 to 100 and 121 to 130.

The cDNAs of the present invention also include cDNA fragments (of 10 bp or more) containing any partial base sequence in the base sequences represented by SEQ ID NOS: 11 to 20, 41 to 50, 71 to 80, 101 to 110 and 131 to 140 or in the base sequences represented by SEQ ID NOS: 21 to 30, 51 to 60, 81 to 90, 111 to 120 and 141 to 150. Also, DNA fragments each consisting of a sense strand and an anti-sense strand shall come within this scope. These DNA fragments can be utilized as the probes for the genetic diagnosis.

The antibody of the present invention can be obtained from a serum after immunizing an animal using the protein of the present invention as an antigen. A peptide that is chemically synthesized based on the amino acid sequence of the present invention and a protein expressed in eukaryotic or prokaryotic cells can be used as an antigen. Alternatively, an antibody can be prepared by introducing the above-mentioned expression vector for eukaryotic cells into the muscle or the skin of an animal by injection or by using a gene gun and then collecting a serum therefrom [JP-A 7-313187]. Animals that can be used include a mouse, a rat, a rabbit, a goat, a chicken and the like. A monoclonal antibody directed to the protein of the present invention can be produced by fusing B cells collected from the spleen of the immunized animal with myelomas to generate hybridomas.

In addition to the activities and uses described above, the polynucleotides and proteins of the present invention may exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E.e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J. E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6-Nordan, R. In Current Protocols in Immunology. J. E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11 -Bennett, F., Giannotti, J., Clark, S. C. and Turner, K. J. In Current Protocols in Immunology. J. E.e.a. Coligan eds. Vol 1 pp. 6.15.1 John Wiley and Sons, Toronto. 1991; Measurement of mouse and human Interleukin 9 -Ciarletta, A., Giannotti, J., Clark, S. C. and Turner, K. J. In Current Protocols in Immunology. J. E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as , for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complementary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium ).

Assays for wound healing activity include, without limitation, those described in: Winter, Epidermal Wound Healing, pps. 71-112 (Maibach, H I and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke)).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or cardiac cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### EXAMPLES

The present invention is specifically illustrated in more detail by the following Examples, but Examples are not intended to restrict the present invention. The basic procedures with regard to the recombinant DNA and the enzymatic reactions were carried out according to the literature [“Molecular Cloning. A Laboratory Manual”, Cold Spring Harbor Laboratory, 1989]. Unless otherwise stated, restriction enzymes and various modifying enzymes to be used were those available from Takara Shuzo. The buffer compositions and the reaction conditions for each of the enzyme reactions were as described in the attached instructions. The cDNA synthesis was carried out according to the literature [Kato, S. et al., Gene 150: 243-250 (1994)].

(1) Selection of cDNAs Encoding Proteins Having Hydrophobic Domains

The cDNA library of epidermoid carcinoma cell line KB (WO98/11217), and the cDNA libraries constructed from human kidney mRNA (Clontech) and human umbilical cord blood mRNA were used as cDNA libraries.

Full-length cDNA clones were selected from the respective libraries and the whole base sequences thereof were determined to construct a homo-protein cDNA bank consisting of the full-length cDNA clones. The hydrophobicity/hydrophilicity profiles were determined for the proteins encoded by the full-length cDNA clones registered in the homo-protein cDNA bank by the Kyte-Doolittle method [Kyte, J. & Doolittle, R. F., J. Mol. Biol. 157: 105-132 (1982)] to examine the presence or absence of a hydrophobic domain. A clone that has a hydrophobic region being assumed as a secretory signal or a transmembrane domain in the amino acid sequence of the encoded protein was selected as a clone candidate.

(2) Protein Synthesis by in vitro Translation

The plasmid vector bearing the cDNA of the present invention was used for in vitro transcription/translation with a T_{N}T rabbit reticulocyte lysate kit (Promega). In this case, [³⁵S]methionine was added to label the expression product with a radioisotope. Each of the reactions was carried out according to the protocols attached to the kit. Two micrograms of the plasmid was subjected to the reaction at 30° C. for 90 minutes in the reaction solution of a total volume of 25 μl containing 12.5 μl μ of T_{N}T rabbit reticulocyte lysate, 0.5 μl of a buffer solution (attached to the kit), 2 μl of an amino acid mixture (without methionine), 2 μl of [³⁵S]methionine (Amersham) (0.37 MBq/μl), 0.5 μl of T7 RNA polymerase, and 20 U of RNasin. The experiment in the presence of a membrane system was carried out by adding 2.5 μl of a canine pancreas microsome fraction (Promega) to the reaction system. 2 μl of the SDS sampling buffer (125 mM Tris-hydrochloride buffer, pH 6.8, 120 mM 2-mercaptoethanol, 2% SDS solution, 0.025% Bromophenol Blue and 20% glycerol) was added to 3 μl of the reaction solution. The resulting mixture was heated at 95° C. for 3 minutes and then subjected to SDS-polyacrylamide gel electrophoresis. The molecular weight of the translation product was determined by carrying out the autoradiography.

(3) Expression in COS7

Escherichia coli cells harboring the expression vector for the protein of the present invention were cultured at 37° C. for 2 hours in 2 ml of the 2×YT culture medium containing 100 μg/ml of ampicillin, the helper phage M13KO7 (50 μl) was added thereto, and the cells were then cultured at 37° C. overnight. Single-stranded phage particles were obtained by polyethylene glycol precipitation from a supernatant separated by centrifugation. The particles were suspended in 100 μl of 1 mM Tris-0.1 mM EDTA, pH 8 (TE).

The cultured cells derived from monkey kidney, COS7, were cultured at 37° C. in the presence of 5% CO₂ in the Dulbecco's modified Eagle's medium (DMEM) containing 10% fetal calf serum. 1×10⁵ COS7 cells were inoculated into a 6-well plate (Nunc, well diameter: 3 cm) and cultured at 37° C. for 22 hours in the presence of 5% CO₂. After the medium was removed, the cell surface was washed with a phosphate buffer solution followed by DMEM containing 50 mM Tris-hydrochloride (pH 7.5) (TDMEM). A suspension containing 1 μl of the single-stranded phage suspension, 0.6 ml of the DMEM medium and 3 μl of TRANSFECTAM™ (IBF) was added to the cells and the cells were cultured at 37° C. for 3 hours in the presence of 5% CO₂. After the sample solution was removed, the cell surface was washed with TDMEM, 2 ml per well of DMEM containing 10% fetal calf serum was added, and the cells were cultured at 37° C. for 2 days in the presence of 5% CO₂. After the medium was exchanged for a medium containing [³⁵S]cysteine or [³⁵S]methionine, the cells were cultured for one hour. After the medium and the cells were separated each other by centrifugation, proteins in the medium fraction and the cell membrane fraction were subjected to SDS-PAGE.

(4) Preparation of Antibodies

A plasmid vector containing the cDNA of the present invention was dissolved in a phosphate buffer solution (PBS: 145 mM NaCl, 2.68 mM KCl, 8.09 mM Na₂HPO₄, 2 mM KH2PO4, pH 7.2) at a concentration of 2 μg/μl. 25 μl each (a total of 50 μl) of the thus prepared plasmid solution in PBS was injected into the right and left musculi quadriceps femoris of three mice (ICR line) using a 26 guage needle. After similar injections were repeated for one month at intervals of one week, blood was collected. The collected blood was stored at 4° C. overnight to coagulate the blood, and then centrifuged at 8,000×g for five minutes to obtain a supernatant. NaN₃ was added to the supernatant to a concentration of 0.01% and the mixture was then stored at 4° C. The generation of an antibody was confirmed by immunostaining of COS7 cells into which the corresponding vector had been introduced, or by Western blotting using a cell lysate or a secreted product.

(5) Clone Examples

<HP03613> (SEQ ID NOS: 1, 11, and 21)

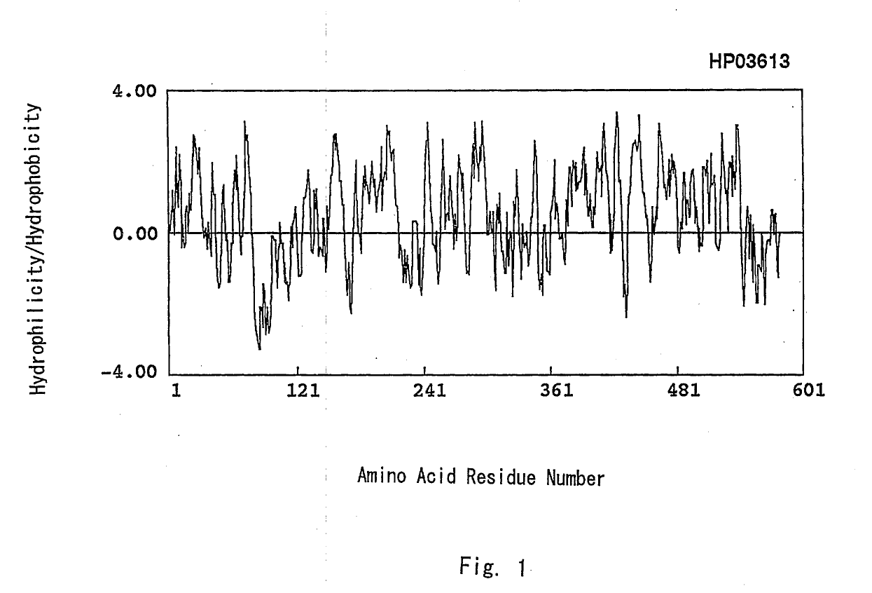

Determination of the whole base sequence of the cDNA insert of clone HP03613 obtained from cDNA library of human kidney revealed the structure consisting of a 337-bp 5′-untranslated region, a 1737-bp ORF, and a 791-bp 3′-untranslated region. The ORF encodes a protein consisting of 578 amino acid residues and there existed eleven putative transmembrane domains. FIG. 1 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to mouse organic cation transporter-like protein (Accession No. BAA23875). Table 2 shows the comparison between amino acid sequences of the human protein of the present invention (HP) and mouse organic cation transporter-like protein (MT). Therein, the marks of -, *, and . represent a gap, an amino acid residue identical with that of the protein of the present invention, and an amino acid residue similar to that of the protein of the present invention, respectively. The both proteins shared a homology of 70.4% in the entire region.

TABLE 2

```
HP MAFSELLDLVGGLGRFQVLQTMALMVSIMWLCTQSMLENFSAAVPSHRCWAPLLDNSTAQ
      ***.**** ********..**.**...*.*. **.********** ****.*******.*
MT MAFPELLDRVGGLGRFQLFQTVALVTPILWVTTQNMLENFSAAVPHHRCWVPLLDNSTSQ
HP ASILGSLSPEALLAISIPPGPNQRPHQCRRFRQPQWQLLDPNATATSWSEADTEPCVDGW
      *** *.*.*..***.******.*.**** ********* ..*****.**.*.**** ***
MT ASIPGDLGPDVLLAVSIPPGPDQQPHQCLRFRQPQWQLTESNATATNWSDAATEPCEDGW
HP VYDRSIFTSTIVAKWNLVCDSHALKPMAWSIYLAGILVGAAACGPASDRFGRRLVLTWSY
      ***.*.* ****..*.***.*.**.******.*********.**.******** ******
MT VYDHSTFRSTIVTTWDLVCNSQALRPMAQSIFLAGILVGAAVCGHASDRFGRRRVLTWSY
HP LQMAVMGTAAAFAPAFPVYCLFRFLLAFAVAGVMMNTGTLRRSLTWRHAGGLHAGSRAEP
      * ..* ****** *.**.********* *********..
MT LLVSVSGTAAFMPTFPLYCLFRFLLASAVAGVMMNTAS----------------------
HP LGLLAVMEWTAARARPLVMTLNSLGFSFGHGLTAAVAYGVRDWTLLQLVVSVPFFLCFLY
                .****.*...*******.******. **..******.* .***.**.**** *.*
MT ----LLMEWTSAQGSPLVMTLNALGFSFGQVLTGSVAYGVRSWRMLQLAVSAPFFLFFVY
HP SWWLAESARWLLTTGRLDWGLQELWRVAAINGKGAVQDTLTPEVLLSAMREELSMGQPPA
      ****.******.*.*.** ***** ****.*. * **** *** *** ** *.... *
MT SWWLPESARWLITVGKLDQGLQELQRVAAVNRRKAEGDTLTMEVLRSAMEEEPSRDKAGA
HP SLGTLLRMPGLRFRTCISTLCWFAFGFTFFGLALDLQALGSNIFLLQMFIGVVDIPAKMG
      ******. **** ** ** **********.***************** .**.**.*.* *
MT SLGTLLGTPGLRGRTIISMLCWFAFGFTFYGLALDLQALGSNIFLLQALIGIVDFPVKTG
HP ALLLLSHLGRRPTLAASLLLAGLCILANTLVPHEMGALRSALAVLGLGGVGAAFTCITLY
      .***.*.**** .. *.*.*****.*.****.**.*********** .*.*******.
MT SLLLISRLGRRLCQVSFLVLPGLCILSNILVPHGMGVLRSALAVLGLGCLGGAFTCITIF
HP SSELFPTVLRMTAVGLGQMAARGGAILGPLVRLLGVHGPWLPLLVYGTVPVLSGLAALLL
      ********.******* *.******.**********.*.*.******.************
MT SSELFPTVIRMTAVGLCQVAARGGAMLGPLVRLLGVYGSWMPLLVYGVVPVLSGLAALLL
HP PETQSLPLPDTIQDVQNQAVKKATHGTLGNSVLKSTQF
      ***..*********.*.*.***.**.*..*.*.**..
MT PETKNLPLPDTIQKIQKQSVKKVTHDTPDGSILMSTRL
```

The search of the GenBank using the base sequences of the present cDNA has revealed the registration of sequences that shared a homology of 90% or more (for example, Accession No. AI792236). However, since they are partial sequences, it can not be judged whether or not they encode the same protein as the protein of the present invention.

<HP03700> (SEQ ID NOS: 2, 12, and 22)

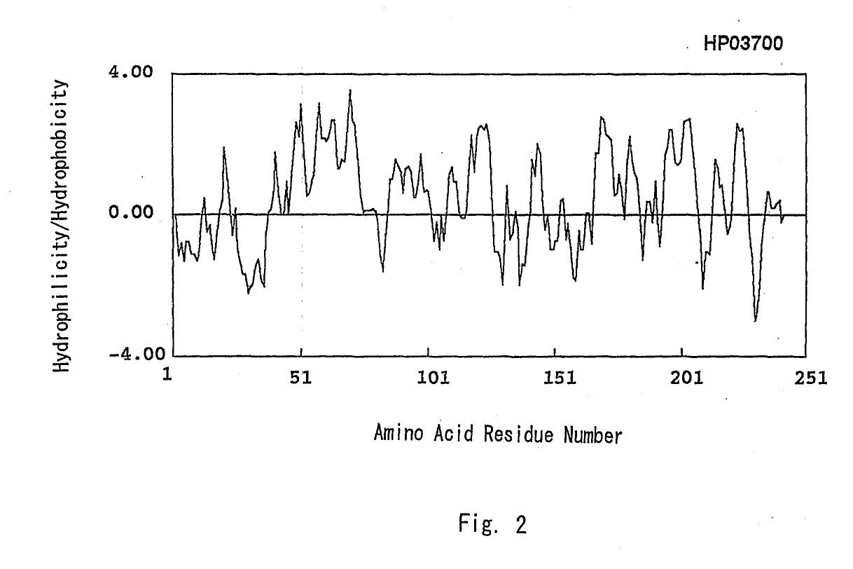

Determination of the whole base sequence of the cDNA insert of clone HP03700 obtained from cDNA library of human kidney revealed the structure consisting of a 45-bp 5′-untranslated region, a 732-bp ORF, and a 2546-bp 3′-untranslated region. The ORF encodes a protein consisting of 243 amino acid residues and there existed three putative transmembrane domains. FIG. 2 depicts the hydrophobicity/hydrophilicity profile, obtained by the Kyte-Doolittle method, of the present protein. In vitro translation resulted in formation of a translation product of 27 kDa that was somewhat larger than the molecular weight of 25,561 predicted from the ORF.

The search of the protein database using the amino acid sequence of the present protein revealed that the protein was similar to mouse yolk sac permease-like molecule 1 (Accession No. AAA92292). Table 3 shows the comparison between amino acid sequences of the human protein of the present invention (HP) and mouse yolk sac permease-like molecule 1 (MY). Therein, the marks of -, *, and . represent a gap, an amino acid residue identical with that of the protein of the present invention, and an amino acid residue similar to that of the protein of the present invention, respectively. The both proteins shared a homology of 74.5% in the N-terminal region of 231 amino acid residues.

TABLE 3

```
HP MSRSPLNPSQLRSVGSQDALAPLPP--PAPQNPSTHSWDP-LCGSLPWGLSCLLALQHVL
      ******.* .* * * **. ***** *. ****..**.. . *. .*********** *
MY MSRSPLHPIPLLSEGYQDTPAPLPPLLPPLQNPSSRSWASRVFGPSTWGLSCLLALQHFL
HP VMASLLCVSHLLLLCSLSPGGLSYSPSQLLASSFFSCGMSTILQTWMGSRLPLVQAPSLE
      *.**** .****** .*.******.*.***********.**.***********.******
MY VLASLLWASHLLLLHGLPPGGLSYPPAQLLASSFFSCGLSTVLQTWMGSRLPLIQAPSLE
HP FLJPALVLTSQKLPRA IQTPGNSSLMLHLCR-GPSCHGLGHWNTSLQEVSGAVVVSGLLQ
      *********.**** ...****.** *.**. ..*****. *****.*************
MY FLIPALVLTNQKLPLTTKTPGNASLSLPLCSLTRSCHGLELWNTSLREVSGAVVVSGLLQ
HP GMMGLLGSPGHVFPHCGPLVLAPSLVVAGLSAHREVAQFCFTHWGLALLYVJSPERRGMVP
      * .**** **.***.******************.******.******* .
MY GTIGLLGVPGRVFPYCGPLVLAPSLVVAGLSAHKEVAQFCSAHWGLALLLILLMVVCSQH
HP SGGVWGD
MY LGSCQIPLCSWRPSSTSTHICIPVFRLLSVLAPVACVWFISAFVGTSVIPLQLSEPSDAP
```

The search of the GenBank using the base sequences of the present cDNA has revealed the registration of sequences that shared a homology of 90% or more (for example, Accession No. AW167520). However, since they are partial sequences, it can not be judged whether or not they encode the same protein as the protein of the present invention.

<HP03935> (SEQ ID NOS: 3, 13, and 23)

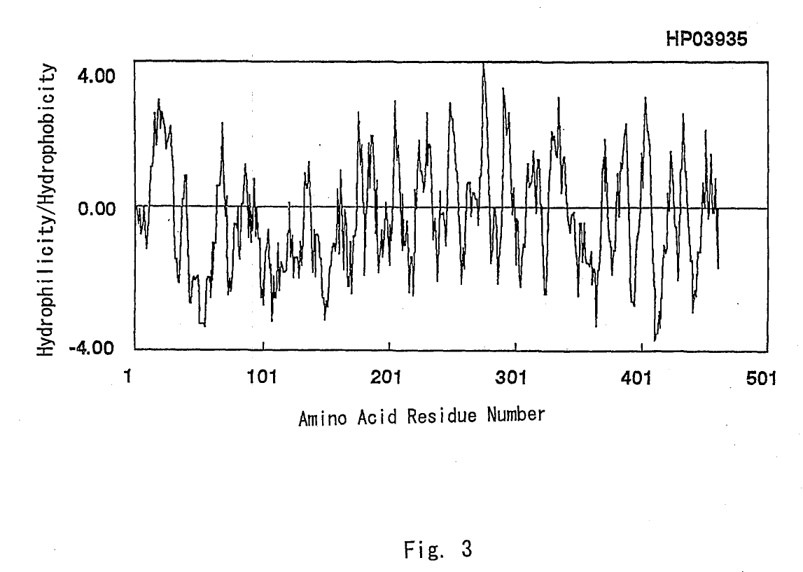

Determination of the whole base sequence of the cDNA insert of clone HP03935 obtained from cDNA library of human kidney revealed the structure consisting of a 72-bp 5′-untranslated region, a 1386-bp ORF, and a 127-bp 3′-untranslated region. The ORF encodes a protein consisting of 461 amino acid residues and there existed a putative secretory signal at the N-terminus. FIG. 3 depicts the hydrophobicity/hydrophilicity profile, obtained by the Kyte-Doolittle method, of the present protein. In vitro translation resulted in formation of a translation product of 56 kDa that was somewhat larger than the molecular weight of 52,052 predicted from the ORF. In this case, the addition of a microsome led to the formation of a product of 61 kDa. In addition, there exists in the amino acid sequence of this protein two sites at which N-glycosylation may occur (Asn-Ser-Ser at position 193 and Asn-Ser-Thr at position 236). Application of the (-3,-1) rule, a method for predicting the cleavage site of the secretory signal sequence, allows to expect that the mature protein starts from histidine at position 32.

The search of the protein database using the amino acid sequence of the present protein revealed that the protein was similar to Arabidopsis thaliana hypothetical protein (Accession No. CAB41318). Table 4 shows the comparison between amino acid sequences of the human protein of the present invention (HP) and Arabidopsis thaliana hypothetical protein (AT). Therein, the marks of -, *, and . represent a gap, an amino acid residue identical with that of the protein of the present invention, and an amino acid residue similar to that of the protein of the present invention, respectively. The both proteins shared a homology of 30.8% in the intermediate region of 214 amino acid residues.

TABLE 4

```
HP MAPQSPLSSRMAPLGMLLGLLMAACFTFCLSHQNLKEFALTNPEKSSTKETERKETKAEE
HP ELDAEVLEVFHPTHEWQALQPGQAVPAGSHVRLNLQTGEREAKLQYEDKFRNNLKGKRLD
AT MPTIFFFRYVFLLVVISLVGFSIAEKVNSSGGMVWSSVRDEAELVEDSGVVIGEQDQ
HP INTNTYTSQDLKSALAKFKEGAEMESSKEDKARQAEVKRLFRPIEELKKDFDELNVVIET
                                          . *..... .* .. . .. * .. ***. ..*. . .
AT IDGGFSSLDGMLHWAIGHSDPATLKEAAKDAEKMS-LDELQKRQLELKELVEKLK--MPS
HP DMQIMVRLINKFNSSSSSLEEKIAALFDLEYYVHQMDNAQDLLSFGGLQVVINGLNSTEP
      . ..* *...*.** ***.. ** .* *...***.** . ***.** ..** ...
AT NAKLMQIAIDDLNNSSLSLEDRHRALQELLILVEPIDNANDLSKSGGLRVVAGELNHDDT
HP LVKEYAAFVLGAAFSSNPKVQVEAIEGGALQKLLVILATEQPLTAKKKVLFALCSLLRHF
        *.. **.*** * .** ** ...* *** . *. .... . .. *.***...*.*.
AT EVRKLAAWVLGKASQNNPFVQEQVLELGALTT-LIKMVNSSSTEEAVKALFAVSALIRNN
HP PYAQRQFLKLGGLQVLRTLVQEKGTEV-LAVRVVTLLTYLVTEKMFAEEEAELTQEMSPE
          .* *. * .**..... . .. * ..* *. **..... ..*..**
AT IAGQDLFFAAHGYIMLRDVMNNGSLDMKLRRKAVFLVGDLAESQLQNTEKDELPIFKDRL
HP KLQQYRQVHLLPGLWEQGWCEITAHLLALPEHDAREKVLQTLGVLLTTCRDRYRQDPQLG
AT FLKSVVKLIVVLDLDLQEKALTAIQTLLQLKSIEPQVLKESCGLEEALERMKLQLEESMA
HP RTLASLQAEYQVLASLELQDGEDEGYFQELLGSVNSLLKELR
AT DEYKRDYAADVESIRGEVELIFRQKLGLL
```

The search of the GenBank using the base sequences of the present cDNA has revealed the registration of sequences that shared a homology of 90% or more (for example, Accession No. AW025017) among ESTs. However, since they are partial sequences, it can not be judged whether or not they encode the same protein as the protein of the present invention.

<HP10755> (SEQ ID NOS: 4, 14, and 24)

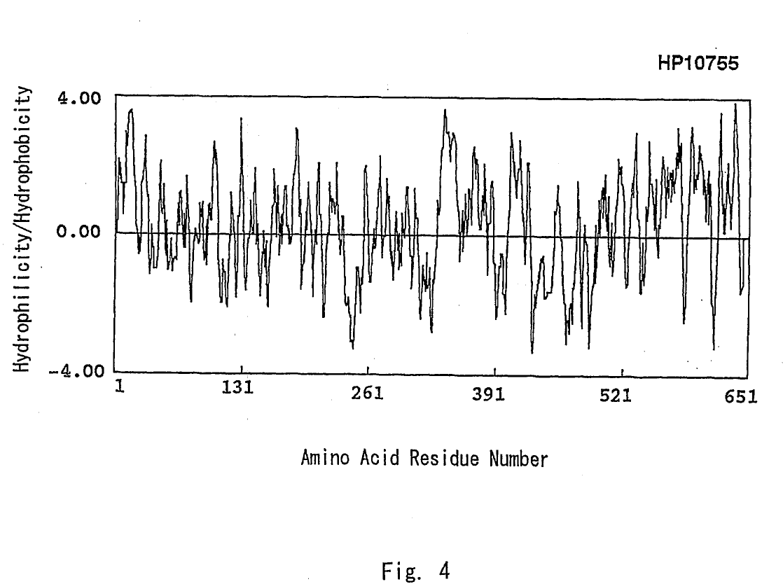

Determination of the whole base sequence of the cDNA insert of clone HP10755 obtained from cDNA library of human kidney revealed the structure consisting of a 55-bp 5′-untranslated region, a 1944-bp ORF, and a 123-bp 3′-untranslated region. The ORF encodes a protein consisting of 647 amino acid residues and there existed eight putative transmembrane domains. FIG. 4 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to human hypothetical protein KIAA0062 (Accession No. BAA06685). Table 5 shows the comparison between amino acid sequences of the human protein of the present invention (HP) and human hypothetical protein KIAA0062 (KI). Therein, the marks of -, *, and . represent a gap, an amino acid residue identical with that of the protein of the present invention, and an amino acid residue similar to that of the protein of the present invention, respectively. The both proteins shared a homology of 30.6% in the C-terminal region of 408 amino acid residues.

TABLE 5

```
HP MASLVSLELGLLAVLVVTATASPPAGLLSLLTSGQGALDQEALGGLLNTLADRVHCTNG
HP PCGKCLSVEDALGLGEPEGSGLPPGPVLEARYVARLSAAAVLYLSNPEGTCEDTRAGLWA
HP SHADHLLALLESPKALTPGLSWLLQRMQARAAGQTPKTACVDIPQLLEEAVGAGAPGSAG
KI RVYADAPAKLLLPPPAAWDLAVRLRGAEAASERQVYSVTM
HP GVLAALLDHVRSGSCFHALPSPQYFVDFVFQQHSSEVPMTLAELSALMQRLGVGREAHSD
KI KLLLLHPAFQSCLLLTLLGLWRTTPEAHASSLGAPAISAASFLQDLIHRYGEGDSLTLQQ
HP HSHRHRGASSRDPVPLISSSNSSSVWDTVCLSARDVMAAYGLSEQAGVTPEAWAQLSPAL
                                                            ..*.*. *...*...***. ..... ....*..
KI LKALLNHLDVGVGRGNVTQHVQGHRNLSTCFSSGDLFTAHNFSEQSRIGSSELQEFCPTI
HP LQQQLSGACTSQSRPPVQDQLSQSER------YLYGSLATLLICLCAVFGLLLLTCTGCR
      *** * ****... ... ....* . ** * . .*.**...* ... .
KI LQQLDSRACTSENQENEENEQTEEGRPSAVEVWGYGLLCVTVISLCSLLGASVVPFMK-K
HP GVAHYILQ2TFLSLAVGALTGDAVLHLTPKVLGLHTHSEEGLSPQPTWRLLAMLAGLYAFF
      . . .* *..**.*.* ..*...*.*...*... *. .... ....*.* **
KI TFYKRLLLYFIALAIGTLYSNALFQLIPEAFGFNPL-EDYYVSKSA----VVFGGFYLFF
HP LFENLFNLLL-PRDPEDLEDGPCGHSS-HSHGGHSHGVSLQLAPSELRQPKPPH----EG
      . *.....** ... .. ...... .* .*. ....** .*. ..* . *.* .
KI FTEKELKILLKQKNEHHHGHSHYASESLPSKKDQEEGVMEKLQNGDLDHMIPQHCSSELD
HP SRADLVAE-------ESPELLNPE-----PRRLS-PELRLLPYMITLGDAVHNFADGLAV
      ..* .*.* . ..* ... . .. ... *..****.*..*** ****.
KI GKAPMVDEKVIVGSLSVQDLQASQSACYWLKGVRYSDIGTLAWMITLSDGLHNFIDGLAI
HP GAAFASSWKTGLATSLAVFCHELPHELGDFAALLHAGLSVRQALLLNLASALTAFAGLYV
      **.*. * *..**.*..*.*.*******. **.**.*..***..*. ** . **
KI GASFTVSVFQGISTSVAILCEEFPHELGDFVILLNAGMSIQQALFFNFLSACCCYLGLAF
HP ALAVGVSEESEAWILAVATGLFLYVALCDMLPAMLKV-----RDPRPWLLFLLHNVGLLG
      .. .* *. *.. **.*.*.*.***..* **.*.* .* *. . . *...*.***.
KI GILAG-SHFSANWIFALAGGMFLYISLADMFPEMNEVCQEDERKGSILIPFIIQNLGLLT
HP GWTVLLLLSLYEDDITF
      *.*....*..*...*
KI GFTIMVVLTMYSGQIQIG
```

Furthermore, the search of the GenBank using the base sequences of the present cDNA has revealed the registration of sequences that shared a homology of 90% or more (for example, Accession No. AA42490) among ESTs. However, since they are partial sequences, it can not be judged whether or not they encode the same protein as the protein of the present invention.

<HP10760> (SEQ ID NOS: 5, 15, and 25)

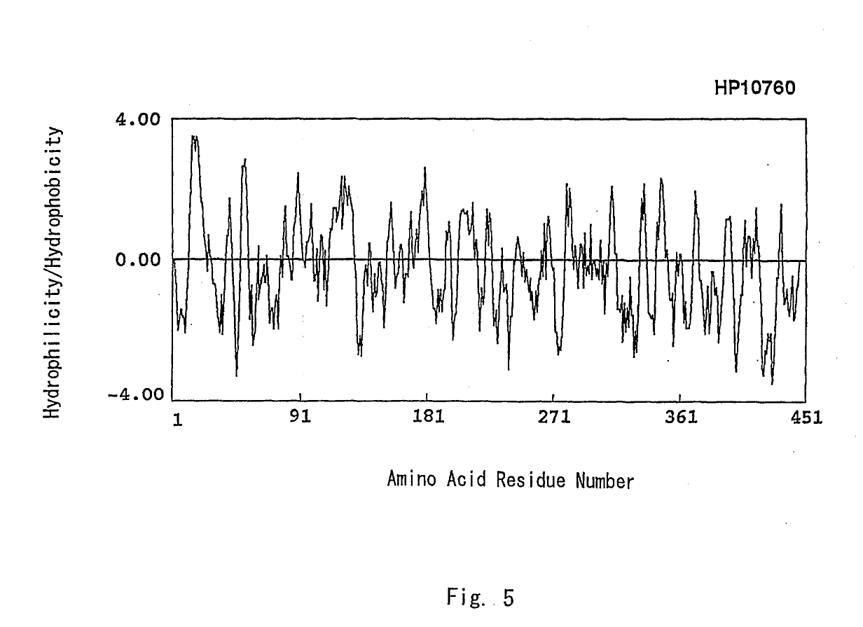

Determination of the whole base sequence of the cDNA insert of clone HP10760 obtained from cDNA library of human kidney revealed the structure consisting of a 61-bp 5′-untranslated region, a 1341-bp ORF, and a 373-bp 3′-untranslated region. The ORF encodes a protein consisting of 446 amino acid residues and there existed a putative secretory signal at the N-terminus. FIG. 5 depicts the hydrophobicity/hydrophilicity profile, obtained by the Kyte-Doolittle method, of the present protein. In vitro translation resulted in formation of a translation product of 48 kDa that was somewhat smaller than the molecular weight of 49,468 predicted from the ORF. In this case, the addition of a microsome led to the formation of a product of 50 kDa. In addition, there exists in the amino acid sequence of this protein two sites at which N-glycosylation may occur (Asn-Ala-Thr at position 144 and Asn-Ile-Ser at position 243). Application of the (-3,-1) rule, a method for predicting the cleavage site of the secretory signal sequence, allows to expect that the mature protein starts from glutamic acid at position 27.

The search of the protein database using the amino acid sequence of the present protein revealed that the protein was similar to human 25 kDa trypsin inhibitor (Accession No. BAA25066). Table 6 shows the comparison between amino acid sequences of the human protein of the present invention (HP) and human 25 kDa trypsin inhibitor (TI). Therein, the marks of -, *, and . represent a gap, an amino acid residue identical with that of the protein of the present invention, and an amino acid residue similar to that of the protein of the present invention, respectively. The both proteins shared a homology of 33.5% in the intermediate region of 185 amino acid residues.

TABLE 6

```
HP MLHPETSPGRGHLLAVLLALLGTAWAEVWPPQLQEQAPMAG
TI MIAISAVSSALLFSLLCEASTVVLLNSTDSSPPTNNFTDIEAALKAQLDSADIPKARRKR
HP ALNRKESFLLLSLHNRLRSWVQPPAADMRRLDWSDSLAQLAQARAALCGIPTPSLASGLW
        ..... . .*. **..*. * ****.* . *...**. *.*.** * * ... .* *
TI YISQNDMIAILDYHNQVRGKVFPPAANMEYMVWDENLAKSAEAWAATC-IWDHG-PSYLL
HP RTLQVGWNMQLLPAGLASFVEVVSLWFAEGQRYSHA-AGEC-----AR--NATCTHYTQL
TI RFLGQN--LSVRTGRYRSILQLVKPWYDEVKDYAFPYPQDCNPRCPMRCFGPMCTHYTQM
HP VWATSSQLGCGRHLCSAGQA--AI---EAF-VCAYSPGGNWEVNGHTIIPYKKGAWCSLC
      *****...**. * * . .. .. ... **.*.* *** *.. *** *. ** *
TI VWATSNRIGCAIGTCQNMNVWGSVWRRAVYLVCNYAPHGNW--IGEA--PYKVGVPCSSC
HP TASVSGCFKAWDHAGGLCEVPRNPCRMSCQNHGRLNISTCHCHCPPGYTGRYCQVRCSLQ
      ..* .*
TI PPSYGGSCTDNLCFPGVTSNYLYWFK
```

The search of the GenBank using the base sequences of the present cDNA has revealed the registration of sequences that shared a homology of 90% or more (for example, Accession No. AI792411) among ESTs. However, since they are partial sequences, it can not be judged whether or not they encode the same protein as the protein of the present invention.

<HP10764> (SEQ ID NOS: 6, 16, and 26)

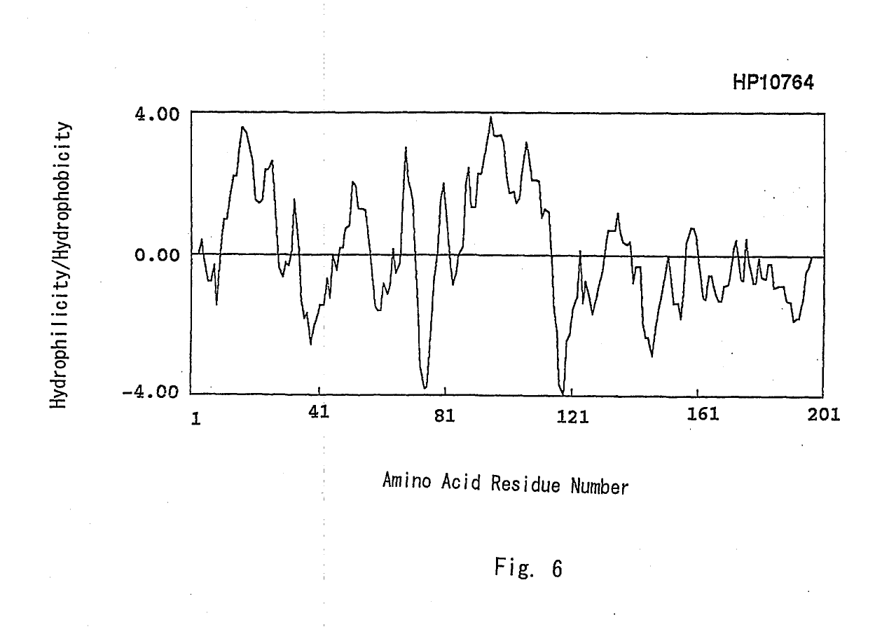

Determination of the whole base sequence of the cDNA insert of clone HP10764 obtained from cDNA library of human kidney revealed the structure consisting of a 326-bp 5′-untranslated region, a 594-bp ORF, and a 452-bp 3′-untranslated region. The ORF encodes a protein consisting of 197 amino acid residues and there existed two putative transmembrane domains. FIG. 6 depicts the hydrophobicity/hydrophilicity profile, obtained by the Kyte-Doolittle method, of the present protein. In vitro translation resulted in formation of a translation product of 25 kDa that was somewhat larger than the molecular weight of 21,508 predicted from the ORF.

The search of the GenBank using the base sequences of the present cDNA has revealed the registration of sequences that shared a homology of 90% or more (for example, Accession No. H45965) among ESTs. However, since they are partial sequences, it can not be judged whether or not they encode the same protein as the protein of the present invention.

<HP10768> (SEQ ID NOS: 7, 17, and 27)

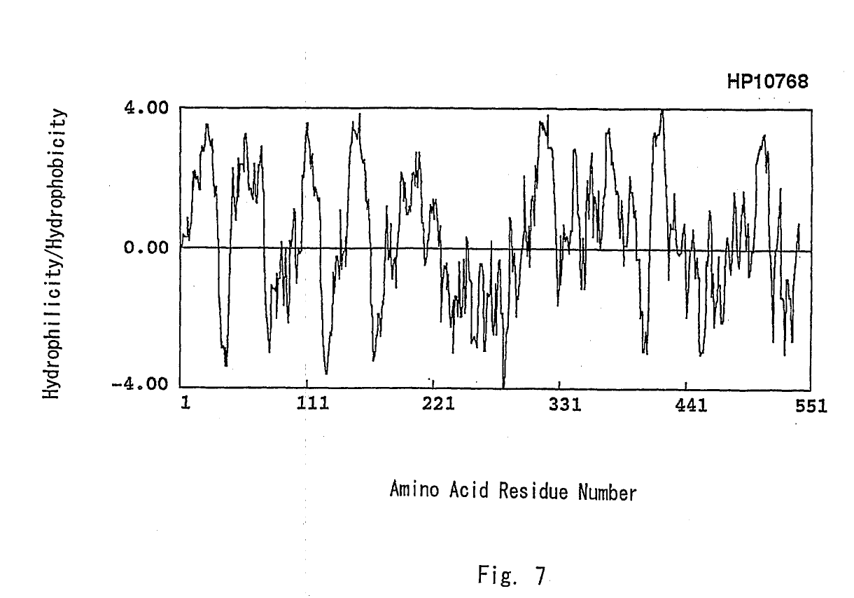

Determination of the whole base sequence of the cDNA insert of clone HP10768 obtained from cDNA library of human kidney revealed the structure consisting of a 100-bp 5′-untranslated region, a 1623-bp ORF, and a 351-bp 3′-untranslated region. The ORF encodes a protein consisting of 540 amino acid residues and there existed nine putative transmembrane domains. FIG. 7 depicts the hydrophobicity/hydrophilicity profile, obtained by the Kyte-Doolittle method, of the present protein. In vitro translation resulted in formation of a translation product of high molecular weight.

The search of the GenBank using the base sequences of the present cDNA has revealed the registration of sequences that shared a homology of 90% or more (for example, Accession No. AA459236) among ESTs. However, since they are partial sequences, it can not be judged whether or not they encode the same protein as the protein of the present invention.

<HP10769> (SEQ ID NOS: 8, 18, and 28)

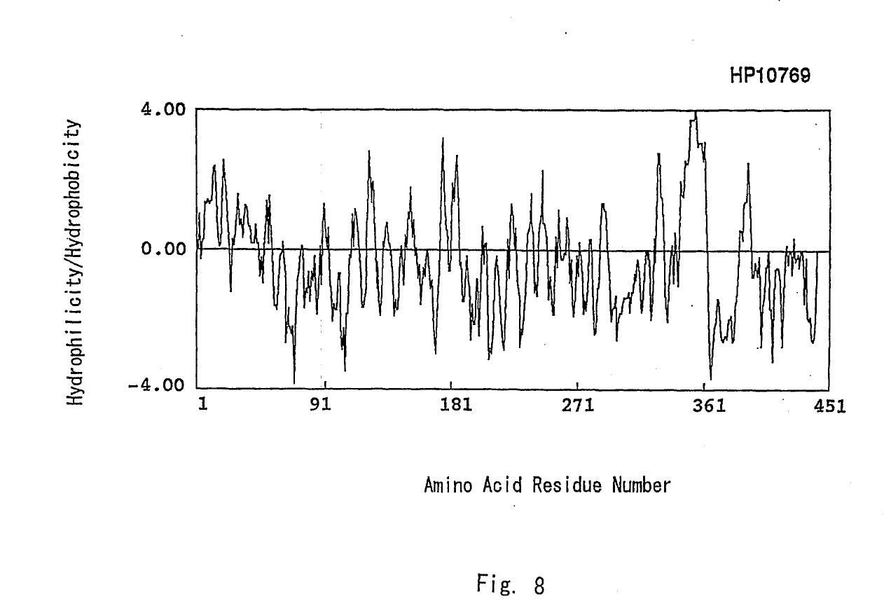

Determination of the whole base sequence of the cDNA insert of clone HP10769 obtained from cDNA library of human kidney revealed the structure consisting of a 11-bp 5′-untranslated region, a 1329-bp ORF, and a 912-bp 3′-untranslated region. The ORF encodes a protein consisting of 442 amino acid residues and there existed two putative transmembrane domains. FIG. 8 depicts the hydrophobicity/hydrophilicity profile, obtained by the Kyte-Doolittle method, of the present protein. In vitro translation resulted in formation of a translation product of 52 kDa that was somewhat larger than the molecular weight of 49,101 predicted from the ORF.

The search of the GenBank using the base sequences of the present cDNA has revealed the registration of sequences that shared a homology of 90% or more (for example, Accession No. AI625881) among ESTs. However, since they are partial sequences, it can not be judged whether or not they encode the same protein as the protein of the present invention.

<HP10784> (SEQ ID NOS: 9, 19, and 29)

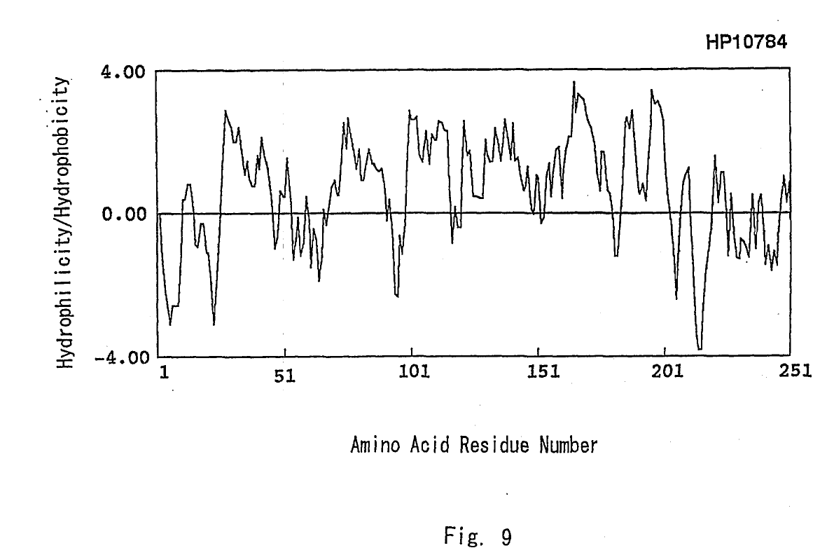

Determination of the whole base sequence of the cDNA insert of clone HP10784 obtained from cDNA library of human kidney revealed the structure consisting of a 60-bp 5′-untranslated region, a 789-bp ORF, and a 612-bp 3′-untranslated region. The ORF encodes a protein consisting of 262 amino acid residues and there existed six putative transmembrane domains. FIG. 9 depicts the hydrophobicity/hydrophilicity profile, obtained by the Kyte-Doolittle method, of the present protein. In vitro translation resulted in formation of a translation product of 28 kDa that was almost identical with the molecular weight of 27,551 predicted from the ORF.

The search of the protein database using the amino acid sequence of the present protein revealed that the protein was similar to rice (Oryza sativa) hypothetical protein (Accession No. AAD39600). Table 7 shows the comparison between amino acid sequences of the human protein of the present invention (HP) and rice hypothetical protein (OS). Therein, the marks of -, *, and . represent a gap, an amino acid residue identical with that of the protein of the present invention, and an amino acid residue similar to that of the protein of the present invention, respectively. The both proteins shared a homology of 40.0% in the intermediate region of 195 amino acid residues.

TABLE 7

```
HP MTPEDPEETQPLLGPPGGSAPRGR
OS MSFRGEESGGEDGGRTASASDLRKPFLGTGSWYKNSSAGGGGGMGSRLGSSAYSLRDSSV
HP RVFLAAFAAALGPLSFGFALGYSSPAIPSLQRAAPPAPRLDDAAASWFGAVVTLGAAAGG
            * .. .****. ***. *.***. .. .. *. .. * **.. ..** .*.
OS SAVLCTLIVALGPIQFGFTCGFSSPTQDAI----ISDLGLTLSEFSLFGSLSNVGAMVGA
HP VLGGWLVDRAGRKLSLLLCSVPFVAGFAVITAAQDVWMLLGGRLLTGLACGVASLVAPVY
      . .* ... *** **.. ..* . *. .*. *.* .*. ****.*.. ** * *.***
OS IASGQIAEYIGRKGSLMIAAIPNIIGWLAISFAKDSSFLFMGRLLEGFGVGVISYVVPVY
HP ISEIAYPAVRGLLGSCVQLMVVVGILLAYLAGWVLEWRWLAVLGCVPPSLMLLLMCFMPE
      *.*** ...** *** ** *..******* * . ** *.*** .* *... . *.**
OS IAEIAPQTMRGALGSVNQLSVTIGILLAYLLGMFVPWRILSVLGILPCSILIPGLFFIPE
HP TPRFLLTQHRRQEAAPGLVRCGHGVQHECLRRLLQADPGWPWQLLARGHLGACLCTAC
      .**.* . .... ...*
OS SPRWLAKMGKMEDFESSLQVLRGFETDIAVEVNEIKRSVQSSRRRTTIRFADIKQKRYSV
```

The search of the GenBank using the base sequences of the present cDNA has revealed the registration of sequences that shared a homology of 90% or more (for example, Accession No. AW028826) among ESTs. However, since they are partial sequences, it can not be judged whether or not they encode the same protein as the protein of the present invention.

<HP10786> (SEQ ID NOS: 10, 20, and 30)

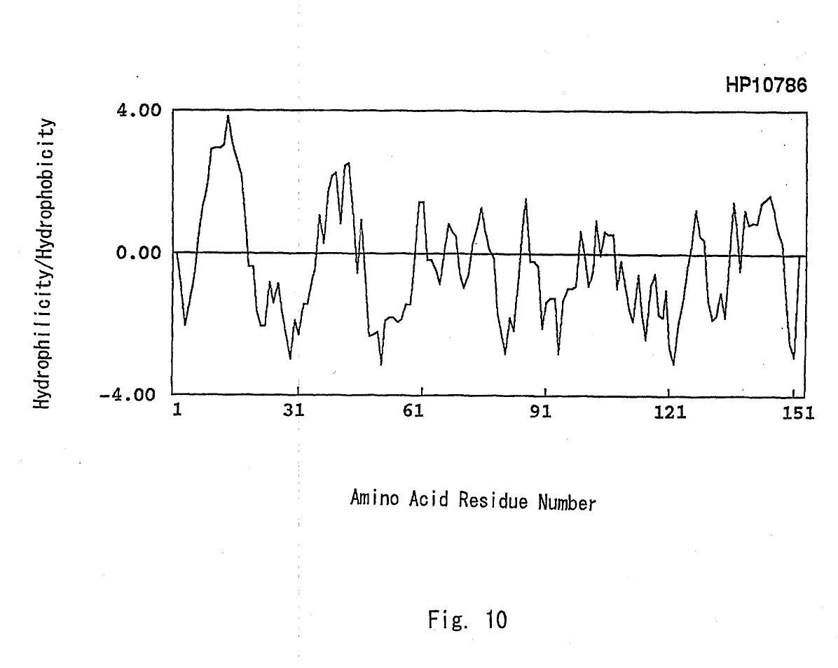

Determination of the whole base sequence of the cDNA insert of clone HP10786 obtained from cDNA library of human kidney revealed the structure consisting of a 78-bp 5′-untranslated region, a 459-bp ORF, and a 585-bp 3′-untranslated region. The ORF encodes a protein consisting of 152 amino acid residues and there existed a putative secretory signal at the N-terminus. FIG. 10 depicts the hydrophobicity/hydrophilicity profile, obtained by the Kyte-Doolittle method, of the present protein. In vitro translation resulted in formation of a translation product of 17 kDa that was almost identical with the molecular weight of 16,904 predicted from the ORF.

Furthermore, the search of the GenBank using the base sequences of the present cDNA has revealed the registration of sequences that shared a homology of 90% or more (for example, Accession No. AW052022) among ESTs. However, since they are partial sequences, it can not be judged whether or not they encode the same protein as the protein of the present invention.

<HP03727> (SEQ ID NOS: 31, 41, and 51)

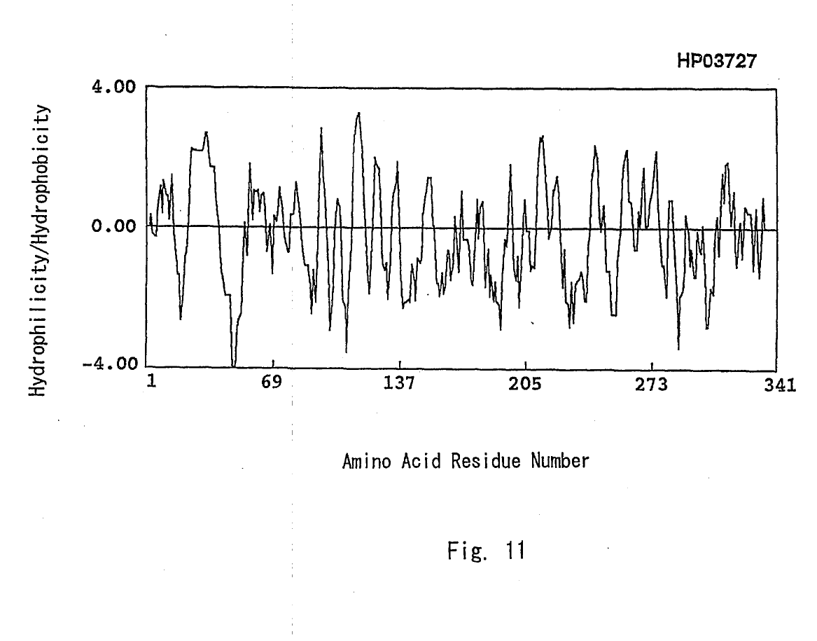

Determination of the whole base sequence of the cDNA insert of clone HP03727 obtained from cDNA library of human kidney revealed the structure consisting of a 254-bp 5′-untranslated region, a 1008-bp ORF, and a 355-bp 3′-untranslated region. The ORF encodes a protein consisting of 335 amino acid residues and there existed one putative transmembrane domain. FIG. 11 depicts the hydrophobicity/hydrophilicity profile, obtained by the Kyte-Doolittle method, of the present protein. In vitro translation resulted in formation of a translation product of 41 kDa that was somewhat larger than the molecular weight of 37,999 predicted from the ORF.

The search of the protein database using the amino acid sequence of the present protein revealed that the protein was similar to protein MG87 from diabetic rat kidney (Accession No. AAC64190). Table 8 shows the comparison between amino acid sequences of the human protein of the present invention (HP) and protein MG87 from diabetic rat kidney (RD). Therein, the marks of -, *, and . represent a gap, an amino acid residue identical with that of the protein of the present invention, and an amino acid residue similar to that of the protein of the present invention, respectively. The both proteins shared a homology of 74.2% in the entire region.

TABLE 8

```
HPMGASSSSALARLGLPARPWPRWLGVAALGLAAVALGTVAWRRAWPRRRRRLQQVGTVAKL
      **.***.********..*.. **********************.*****.*******.*.
RD MGSSSSTALARLGLPGQPRSTWLGVAALGLAAVALGTVAWRRARPRRRRQLQQVGTVSKV
HP WIYPVKSCKGVPVSEAECTAMGLRSGNLRDRFWLVIKEDGHMVTARQEPRLVLISIIYEN
      ****.******.*.*.***.****.*..*****.*.******.**********..*. **
RD WIYPIKSCKGVSVCETECTDMGLRCGKVRDRFWMVVKEDGHMITARQEPRLVLVTITLEN
HP NCLIFRAPDMDQLVLRSKQPSSNKLHNCRIFGLDIKGRDCGNEAAKWFTNFLKTEAYRLV
      *.*.. **.*..*** * *****.*.**.***********.*.*.***..***.*****
RD NYLMLEAPGMEPIVLPIKLPSSNKIHDCRLFGLDIKGRDCGDEVARWFTSYLKTQAYRLV
HP QFETNMKGRTSRKLLPTLD--QNFQVAYPDYCPLLIMTDASLVDLNTRMEKKMKMENFEP
      **.*.*****..** *. . **..*****..*. ....*********..**.*** ***
RD QFDTKMKGRTTKKLYPSESYLQNYEVAYPDCSPIHLISEASLVDLNTRLQKKVKMEYFRP
HP NIVVTGCDAFEEDTWDELLIGSVEVKKVMACPRCILTTVDPDTGVIDRKQPLDTLKSYRL
      ****.**.*************.**.*.*..****.*********.****.**.*******
RD NIVVSGCEAFEEDTWDELLIGDVEMKRVLSCPRCVLTTVDPDTGIIDRKEPLETLKSYRL
HP CDPSERELYKLSPLFGIYYSVEKIGSLRVGDPVYRMV
      **** ..**. *****.*.******************
RD CDPSVKSLYQSSPLFGMYFSVEKIGSLRVGDPVYRMVD
```

The search of the GenBank using the base sequences of the present cDNA has revealed the registration of sequences that shared a homology of 90% or more (for example, Accession No. AI912794) among ESTs. However, since they are partial sequences, it can not be judged whether or not they encode the same protein as the protein of the present invention.

<HP03801> (SEQ ID NOS: 32, 42, and 52)

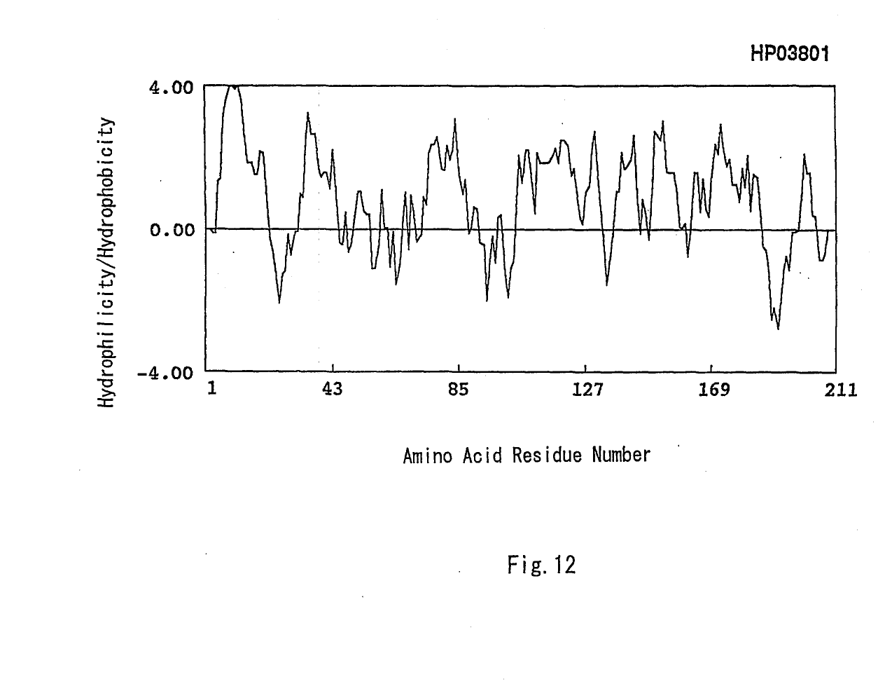

Determination of the whole base sequence of the cDNA insert of clone HP03801 obtained from cDNA library of human umbilical cord blood revealed the structure consisting of a 158-bp 5′-untranslated region, a 627-bp ORF, and a 964-bp 3′-untranslated region. The ORF encodes a protein consisting of 208 amino acid residues and there existed six putative transmembrane domains. FIG. 12 depicts the hydrophobicity/hydrophilicity profile, obtained by the Kyte-Doolittle method, of the present protein. In vitro translation resulted in formation of a translation product of 23 kDa that was almost identical with the molecular weight of 22,526 predicted from the ORF.

The search of the protein database using the amino acid sequence of the present protein revealed that the protein was similar to human hypothetical protein CGI-15 (Accession No. AAD27724). Table 9 shows the comparison between amino acid sequences of the human protein of the present invention (HP) and human hypothetical protein CGI-15 (CP). Therein, the marks of -, *, and . represent a gap, an amino acid residue identical with that of the protein of the present invention, and an amino acid residue similar to that of the protein of the present invention, respectively. The amino acid sequences of the two proteins were completely different each other in the N-terminal, intermediate and C-terminal regions although partial match was observed.

```
TABLE 9
HP MELRAALVLVVLLIAGGLFMFTYKSTQFNVEGFALVLGASFJGGIRW
                                    ********************************* *.. ..
CP VLFIL TFSL TFKLEELRAALVLVVLLIAGGLFMFTYKSTQFNVEGFAWCWGPRSSVAFAG
HP TLTQMLLQKAELGLQNPIDTMFHLQPLMFLGLFPLFAVFEGLHLSTSEKIFRFQDTGLLL
      . .. .. . . . * .. . ******************************
CP PSPRCSCRRLNSASRJPSTPCSTCSHSCSWGLFPLFAVFEGLHLSTSEKJFRFQDTGLLL
HP RVLGSLFLGGILAFGLGFSEFLLVSRTSSLTLSIAGIFKEVCTLLLAAHLLGDQISLLNW
      ************************************************************
CP RVLGSLFLGGILAFGLGFSEFLLVSRTSSLTLSIAGIFKEVCTLLLAAHLLGDQISLLNW
HP LGFALCLSGJSLHVALKALHSRGNPESLPEASVFCSSPCDS
      ****
CP LGFASASREYPSTLPSKPCIPEVMVAPRP
```

Furthermore, the search of the GenBank using the base sequences of the present cDNA has revealed the registration of sequences that shared a homology of 90% or more (for example, Accession No. AI741613) among ESTs. However, since they are partial sequences, it can not be judged whether or not they encode the same protein as the protein of the present invention.

<HP03883> (SEQ ID NOS: 33, 43, and 53)

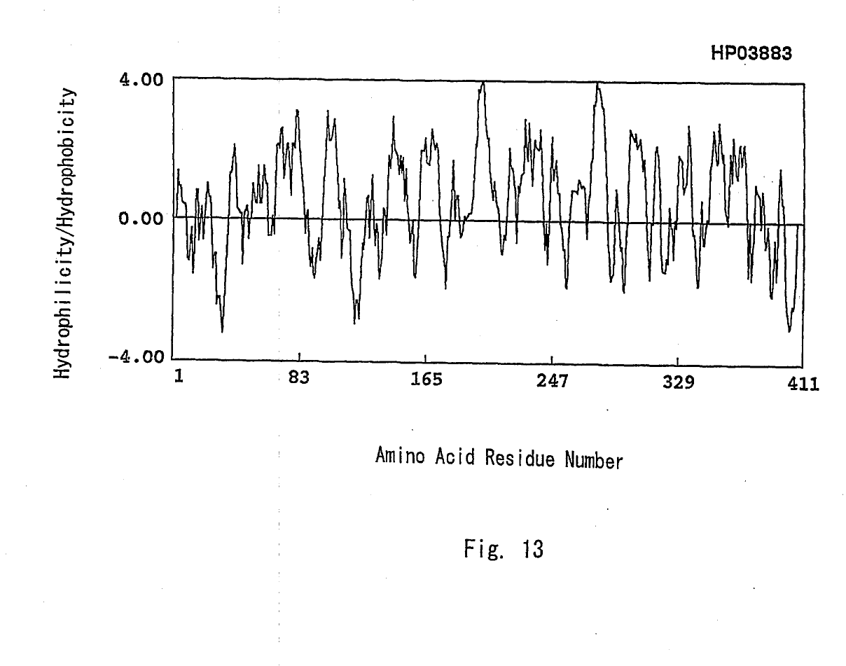

Determination of the whole base sequence of the cDNA insert of clone HP03883 obtained from cDNA library of human kidney revealed the structure consisting of a 59-bp 5′-untranslated region, a 1221-bp ORF, and a 122-bp 3′-untranslated region. The ORF encodes a protein consisting of 406 amino acid residues and there existed eight putative transmembrane domains. FIG. 13 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to human choline/ethanolamine phosphotransferase (Accession No. NP_{—}006081). Table 10 shows the comparison between amino acid sequences of the human protein of the present invention (HP) and human choline/ethanolamine phosphotransferase (CE). Therein, the marks of -, *, and . represent a gap, an amino acid residue identical with that of the protein of the present invention, and an amino acid residue similar to that of the protein of the present invention, respectively. The both proteins shared a homology of 66.8% in the entire region. In addition, the amino acid sequence from position 70 to position 311 of the present protein shared a homology of 98.3% with human AAPT1-like protein (Accession No. AAD44019).

TABLE 10

```
HP  MAAGAGAGSAPRWLRALSEPLSAAQLRRLEEHRYSAAG
                                                                                          *** **.******* .**
CE MSGHRSTRKRCGDSHPESPVGFGHMSTTGCVLNKLFQLPTPPLSRHQLKRLEEHRYQSAG
HP VSLLEPPLQLYWTWLLQWIPLWMAPNSITLLGLAVNVVTTLVLISYCPTATEEAPYWTYL
        ***** .* **.**....* *.*** **..**..*. **..*. *******.** *.*.
CE RSLLEPLMQGYWEWLVRRVPSWIAPNLITIIGLSINICTTILLVFYCPTATEQAPLWAYI
HP LCALGLFIYQSLDAIDGKQARRTNSCSPLGELFDHGCDSLSTVFMAVGASIAARLGTYPD
        ** *********************.******************...*..**..*** **
CE ACACGLFIYQSLDAIDGKQARRTNSSSPLGELFDHGCDSLSTVFVVLGTCIAVQLGTNPD
HP WFFFCSFIGMFVFYCAHWQTYVSGMLRFGKVDVTEIQIALVIVFVLSAFGGATMWDYTIP
      *.***.* * *.************ **** . ****. ** ..*. .*...**...*. **
CE WMFFCCFAGTFMFYCAHWQTYVSGTLRFGIIDVTEVQIFIIIMHLLAVIGGPPFWQSMIP
HP ILEIKLKILPVLGFLGGVIFSCSNYFHVILHGGVGKNGSTIAGTSVLSPGLHIGLIIILA
      .*.*..**.*.* ..*.****.***.**. ****************** **** .*.**
CE VLNIQMKIFPALCTVAGTIFSCTNYFRVIFTGGVGKNGSTIAGTSVLSPFLHIGSVITLA
HP IMIYKKSATDVFEKHPCLYILMFGCVFAKVSQKLVVAHMTKSELYLQDTVFLGPGLLFLD
      *******...********** ** * **...***********..*.**.*.**.*****
CE AMIYKKSAVQLFEKHPCLYILTFGFVSAKITNKLVVAHMTKSEMHLHDTAFIGPALLFLD
HP QYFNNFIDEYVVLWMAMVISSFDMVIYFSALCLQISRHLHLNIFKTACHQAPEQVQVLSS
      ****.*****.***.*.*.* **.. * ..* **..***...*.
CE QYFNSFIDEYIVLWIALVFSFFDLIRYCVSVCNQIASHLHIHVFRIKVSTAHSNHH
```

The search of the GenBank using the base sequences of the present cDNA has revealed the registration of sequences that shared a homology of 90% or more (for example, Accession No. AI816449) among ESTs. However, since they are partial sequences, it can not be judged whether or not they encode the same protein as the protein of the present invention.

<HP03913> (SEQ ID NOS: 34, 44, and 54)

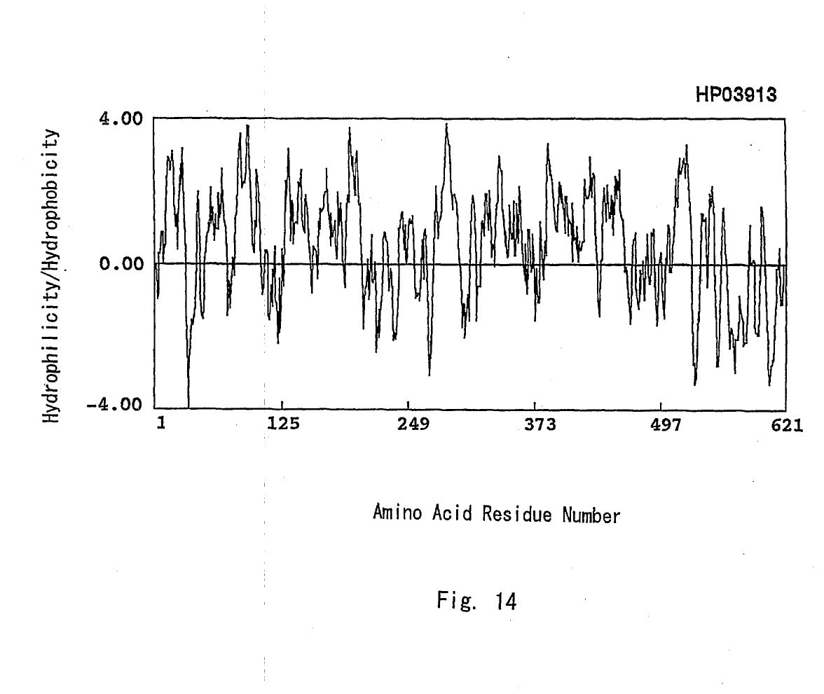

Determination of the whole base sequence of the cDNA insert of clone HP03913 obtained from cDNA library of human kidney revealed the structure consisting of a 344-bp 5′-untranslated region, a 1857-bp ORF, and a 273-bp 3′-untranslated region. The ORF encodes a protein consisting of 618 amino acid residues and there existed thirteen putative transmembrane domains. FIG. 14 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to human solute carrier family 5 (Accession No. NP_{—}000444). Table 11 shows the comparison between amino acid sequences of the human protein of the present invention (HP) and human solute carrier family 5 (SC). Therein, the marks of -, *, and . represent a gap, an amino acid residue identical with that of the protein of the present invention, and an amino acid residue similar to that of the protein of the present invention, respectively. The both proteins shared a homology of 48.3% in the entire region.

TABLE 11

```
HP MEVKNFAVWDYVVFAALFFISSGIGVFFAIKERKKATSREFLVGGRQMSFGPVG
                    * .*..*** ***....*.***.. .. . .. .*..***... ***
SC MEAVETGERPTFGAWDYGVFALMLLVSTGIGLWVGLARGGQRSAEDFFTGGRRLAALPVG
HP LSLTASFMSAVTVLGTPSEVYRFGASFLVFFIAYLFVILLTSELFLPVFYRSGITSTYEY
      ***.******* ***.***.**.* .** . .. *. .**. **.***** *.******
SC LSLSASFMSAVQVLGVPSEAYRYGLKFLWMCLGQLLNSVLTALLFMPVFYRLGLTSTYEY
HP LQLRFNKPVRYAATVIYIVQTILYTGVVVYAPALALNQVTGFDLWGSVFATGIVCTFYCT
      *..**...** .*. ***.*.****.*.***** ******.*.*.*...***.**** .
SC LEMRFSRAVRLCGTLQYIVATMLYTGIVIYAPALILNQVTGLDIWASLLSTGIICTFYTA
HP LGGLKAVVWTDAFQMVVMIVGFLTVLIQGSTHAGGFHNVLEQSTNGSRLHIFDFDVDPLR
      .**.*******.**.***. ** . ** .* .** ..**. . * **....**. ** .
SC VGGMKAVVWTDVFQVVVMLSGFWVVLARGVMLVGGPRQVLTLAQNHSRINLMDFNPDPRS
HP RHTFWTITVGGTFTWLGIYGVNQSTIQRCISCKTEKHAKLALYFNLLGLWIILVCAVFSG
      *.****..****..**..*****. .**...*.***.***** .* . **..*. .*. .*
SC RYTFWTFVVGGTLVWLSMYGVNQAQVQRYVACRTEKQAKLALLINQVGLFLIVSSAACCG
HP LIMYSHFKDCDPWTSGIISAPDQLMPYFVMEIFATMPGLPGLFVACAFSGTLSTVASSIN
      ..*. ..**** * ****** ** .*..**...**.****.***.******...***
SC IVMFVFYTDCDPLLLGRISAPDQYMPLLVLDIFEDLPGVPGLFLACAYSGTLSTASTSIN
HP ALATVTFEDFVKSCFPHLSDKLSTWISKGLCLLFGVMCTSMAVAASVM-GGVVQASLSIH
      *.*.** **..*. .. *. . . *****.*..* * ..*. .*.. ***.*.*...
SC AMAAVTVEDLIKPRLRSLAPRKLVIISKGLSLIYGSACLTVAALSSLLGGGVLQGFTVM
HP GMCGGPMLGLFSLGIVFPFVNWKGALGGLLTGITLSFWNAIGAFIYPAPASKTWPLPLST
      *. .**.** * **. .* * *.*.** .*..**.***.** .**... . . ** *.
SC GVISGPLLGAFILGMFLPACNTPGVLAGLGAGLALSLWVALGATLYPPSEQTMRVLPSSA
HP DQCIKSNVTATG---PPVL--------------SSRPGIADTWYSISYLYYSAVGCLGCI
      ..*. .*.*.* *..* .***..**..*.******.*.* *. .
SC ARCVALSVNASGLLDPALLPANDSSRAPSSGMDASRPALADSFYAISYLYYGALGTLTTV
HP VAGVIISLITGRQRGEDIQPLLIRPVCNLFCFWSKKYKTLCWCGVQHDSGTEQENLENGS
      . *..** .**. . ....* *..
SC LCGALISCLTGPTKRSTLAPGLLWWDLARQTASVAPKEEVAILDDNLVKGPEELPTGNKK
HP ARKQGAESVLQNGLRRESLVHVPGYDPKDKSYNNMAFETTHF
SC PPGFLPTNEDRLFFLGQKELEGAGSWTPCVGHDGGRDQQETNL
```

The search of the GenBank using the base sequences of the present cDNA has revealed the registration of sequences that shared a homology of 90% or more (for example, Accession No. AI733508) among ESTs. However, since they are partial sequences, it can not be judged whether or not they encode the same protein as the protein of the present invention.

<HP10753> (SEQ ID NOS: 35, 45, and 55)

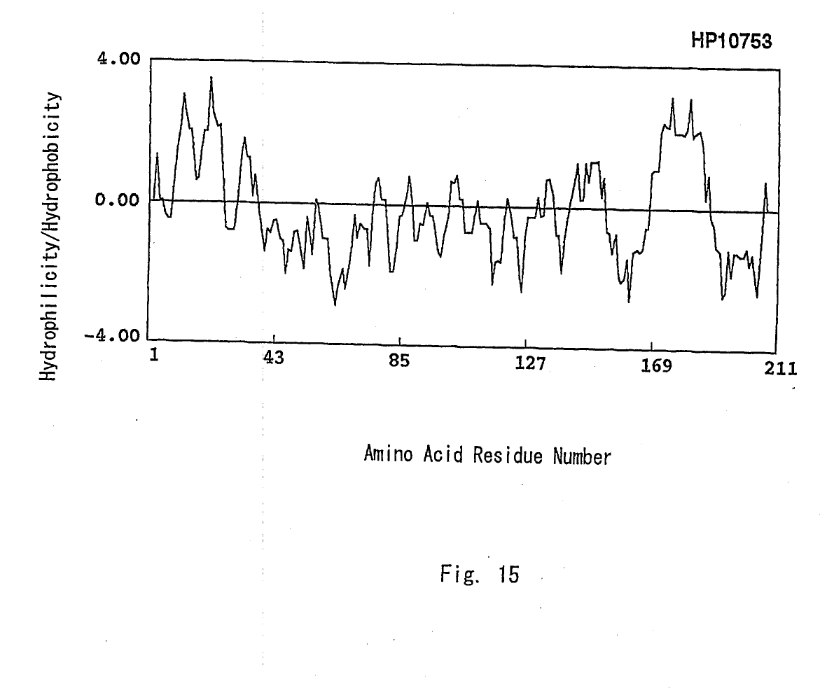

Determination of the whole base sequence of the cDNA insert of clone HP10753 obtained from cDNA library of human umbilical cord blood revealed the structure consisting of a 141-bp 5′-untranslated region, a 627-bp ORF, and a 2528-bp 3′-untranslated region. The ORF encodes a protein consisting of 208 amino acid residues and there existed a putative secretory signal at the N-terminus and one putative transmembrane domain in the inner portion. FIG. 15 depicts the hydrophobicity/hydrophilicity profile, obtained by the Kyte-Doolittle method, of the present protein. In vitro translation resulted in formation of a translation product of 28 kDa that was somewhat larger than the molecular weight of 21,518 predicted from the ORF. Application of the (-3,-1) rule, a method for predicting the cleavage site of the secretory signal sequence, allows to expect that the mature protein starts from methionine at position 32.

The search of the GenBank using the base sequences of the present cDNA has revealed the registration of sequences that shared a homology of 90% or more (for example, Accession No. AW162064) among ESTs. However, since they are partial sequences, it can not be judged whether or not they encode the same protein as the protein of the present invention.

<HP10758> (SEQ ID NOS: 36, 46, and 56)

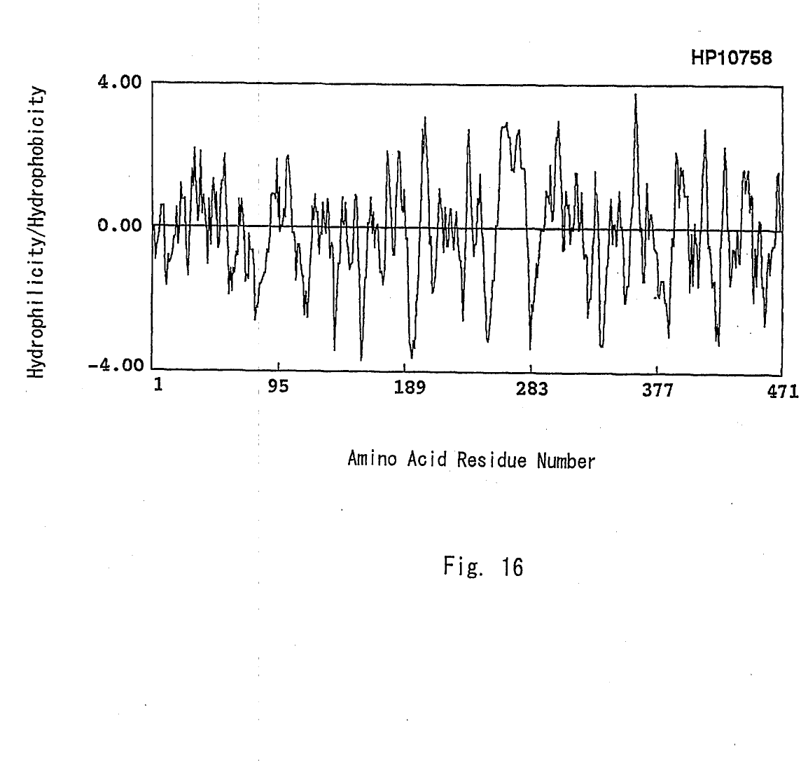

Determination of the whole base sequence of the cDNA insert of clone HP10758 obtained from cDNA library of human kidney revealed the structure consisting of a 25-bp 5′-untranslated region, a 1509-bp ORF, -and a 284-bp 3′-untranslated region. The ORF encodes a protein consisting of 502 amino acid residues and there existed a putative secretory signal at the N-terminus and one putative transmembrane domain in the inner portion. FIG. 16 depicts the hydrophobicity/hydrophilicity profile, obtained by the Kyte-Doolittle method, of the present protein. In vitro translation resulted in formation of a translation product of 60 kDa that was somewhat larger than the molecular weight of 55,848 predicted from the ORF. In this case, the addition of a microsome led to the formation of a product of 66 kDa. In addition, there exists in the amino acid sequence of this protein six sites at which N-glycosylation may occur (Asn-Val-Ser at position 67, Asn-Tyr-Thr at position 103, Asn-Phe-Thr at position 156, Asn-Ile-Thr at position 183, Asn-Phe-Thr at position 197 and Asn-Lys-Ser at position 283). Application of the (-3,-1) rule, a method for predicting the cleavage site of the secretory signal sequence, allows to expect that the mature protein starts from alanine at position 15.

The search of the GenBank using the base sequences of the present cDNA has revealed the registration of sequences that shared a homology of 90% or more (for example, Accession No. T96740) among ESTs. However, since they are partial sequences, it can not be judged whether or not they encode the same protein as the protein of the present invention.

<HP10771> (SEQ ID NOS: 37, 47, and 57)

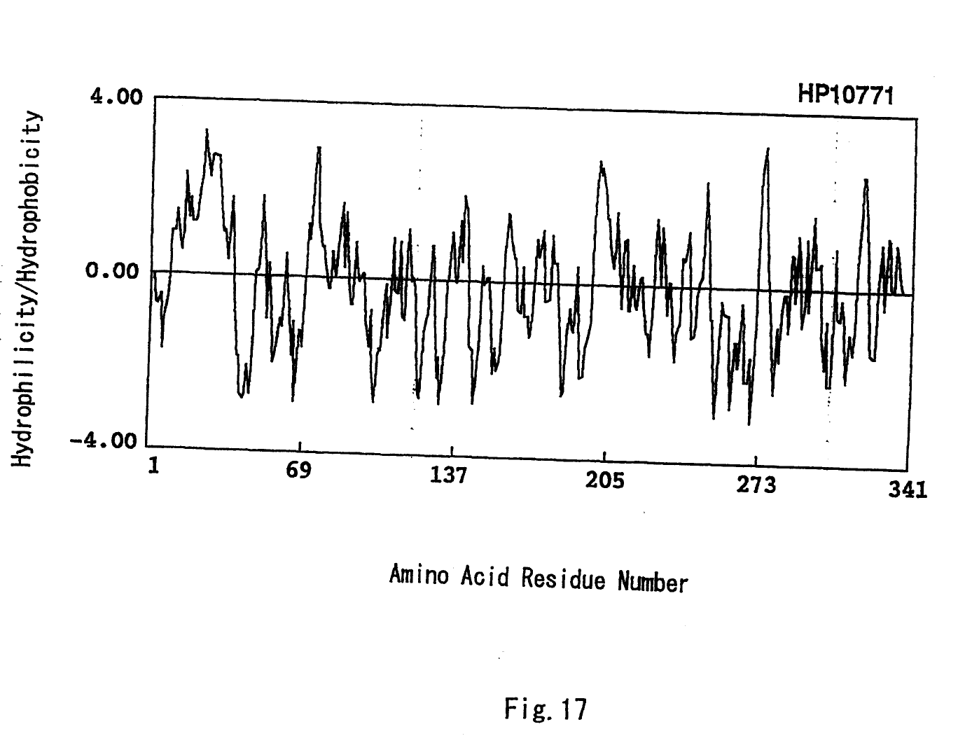

Determination of the whole base sequence of the cDNA insert of clone HP10771 obtained from cDNA library of human kidney revealed the structure consisting of a 36-bp 5′-untranslated region, a 1011-bp ORF, and a 599-bp 3′-untranslated region. The ORF encodes a protein consisting of 336 amino acid residues and there existed one putative transmembrane domain at the N-terminus. FIG. 17 depicts the hydrophobicity/hydrophilicity profile, obtained by the Kyte-Doolittle method, of the present protein. In vitro translation resulted in formation of a translation product of 41 kDa that was somewhat larger than the molecular weight of 37,924 predicted from the ORF.

The search of the protein database using the amino acid sequence of the present protein revealed that the protein was similar to human interferon-α induced protein (Accession No. AR053364). The C-terminal portion downstream from methionine at position 51 of the protein of the present invention matched with the C-terminal portion downstream from methionine at position 12 of human interferon-α induced protein. However, the putative transmembrane domain at the N-terminus observed for the protein of the present invention was not present in human interferon-α induced protein.

The search of the GenBank using,the base sequences of the present cDNA has revealed the registration of sequences that shared a homology of 90% or more (for example, Accession No. AA452543) among ESTs. However, since they are partial sequences, it can not be judged whether or not they encode the same protein as the protein of the present invention.

<HP10778> (SEQ ID NOS: 38, 48, and 58)

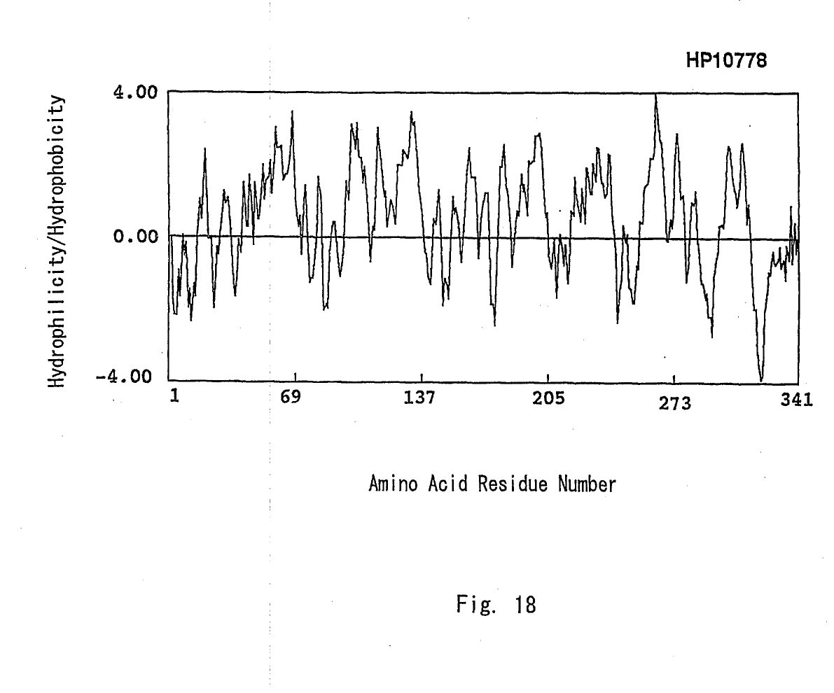

Determination of the whole base sequence of the cDNA insert of clone HP10778 obtained from cDNA library of human kidney revealed the structure consisting of a 173-bp 5′-untranslated region, a 1023-bp ORF, and a 220-bp 3′-untranslated region. The ORF encodes a protein consisting of 340 amino acid residues and there existed six putative transmembrane domains. FIG. 18 depicts the hydrophobicity/hydrophilicity profile, obtained by the Kyte-Doolittle method, of the present protein. In vitro translation resulted in formation of a translation product of high molecular weight.

The search of the GenBank using the base sequences of the present cDNA has revealed the registration of sequences that shared a homology of 90% or more (for example, Accession No. AA429745) among ESTs. However, since they are partial sequences, it can not be judged whether or not they encode the same protein as the protein of the present invention.

<HP10781> (SEQ ID NOS: 39, 49, and 59)

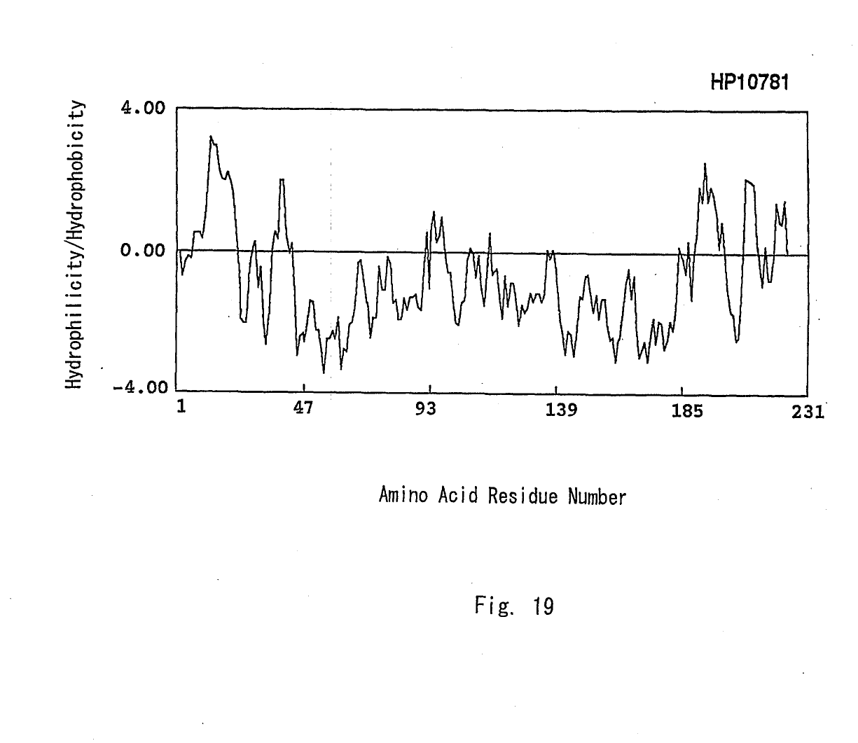

Determination of the whole base sequence of the cDNA insert of clone HP10781 obtained from cDNA library of human kidney revealed the structure consisting of a 88-bp 5′-untranslated region, a 672-bp ORF, and a 1167-bp 3′-untranslated region. The ORF encodes a protein consisting of 223 amino acid residues and there existed a putative secretory signal at the N-terminus. FIG. 19 depicts the hydrophobicity/hydrophilicity profile, obtained by the Kyte-Doolittle method, of the present protein. In vitro translation resulted in formation of a translation product of 31 kDa that was larger than the molecular weight of 24,239 predicted from the ORF. In this case, the addition of a microsome led to the formation of a product of 33 kDa. In addition, there exists in the amino acid sequence of this protein two sites at which N-glycosylation may occur (Asn-Asn-Thr at position 70 and Asn-Thr-Ser at position 71). Application of the (-3,-1) rule, a method for predicting the cleavage site of the secretory signal sequence, allows to expect that the mature protein starts from gluthamine at position 23.

The search of the GenBank using the base sequences of the present cDNA has revealed the registration of sequences that shared a homology of 90% or more (for example, Accession No. AA334609) among ESTs. However, since they are partial sequences, it can not be judged whether or not they encode the same protein as the protein of the present invention.

<HP10785> (SEQ ID NOS: 40, 50, and 60)

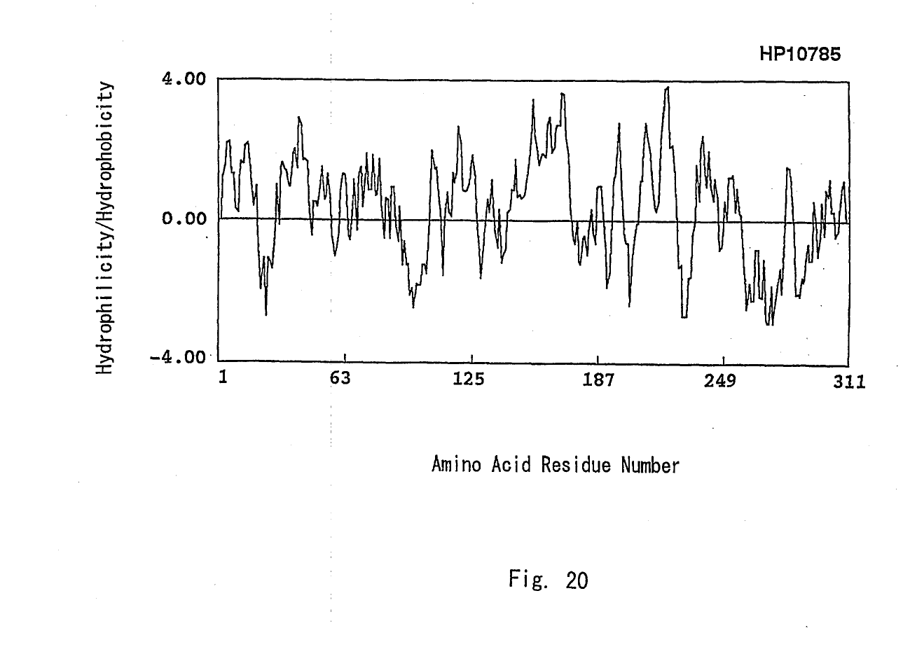

Determination of the whole base sequence of the cDNA insert of clone HP10785 obtained from cDNA library of human kidney revealed the structure consisting of a 171-bp 5′-untranslated region, a 930-bp ORF, and a 318-bp 3′-untranslated region. The ORF encodes a protein consisting of 309 amino acid residues and there existed six putative transmembrane domains. FIG. 20 depicts the hydrophobicity/hydrophilicity profile, obtained by the Kyte-Doolittle method, of the present protein. In vitro translation resulted in formation of a translation product of high molecular weight.

Furthermore, the search of the GenBank using the base sequences of the present cDNA has revealed the registration of sequences that shared a homology of 90% or more (for example, Accession No. AI822041) among ESTs. However, since they are partial sequences, it can not be judged whether or not they encode the same protein as the protein of the present invention.

<HP03878>(SEQ ID NOS: 61, 71, and 81)

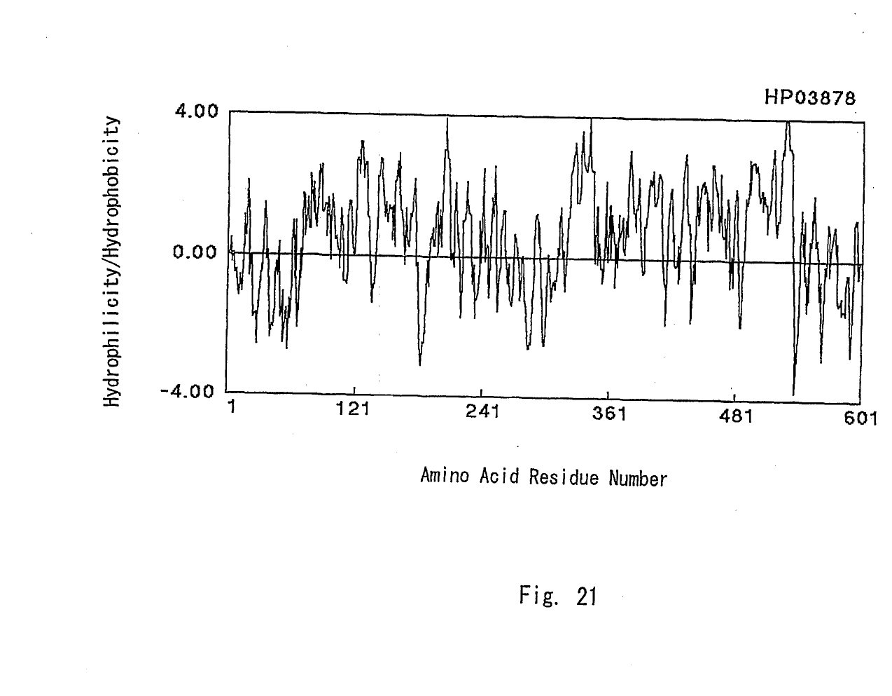

Determination of the whole base sequence of the cDNA insert of clone HP03878 obtained from cDNA library of human kidney revealed the structure consisting of a 77-bp 5′-untranslated region, a 1800-bp ORF, and a 139-bp 3′-untranslated region. The ORF encodes a protein consisting of 599 amino acid residues and there existed ten putative transmembrane domains. FIG. 21 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to flounder (Pseudopleuronectes americanus) Na/Pi cotransport system protein (Accession No. AAB16821). Table 12 shows the comparison between amino acid sequences of the human protein of the present invention (HP) and flounder Na/Pi cotransport system protein (PN). Therein, the marks of -, *, and . represent a gap, an amino acid residue identical with that of the protein of the present invention, and an amino acid residue similar to that of the protein of the present invention, respectively. The both proteins shared a homology of 57.1% in the region of 545 amino acid residues other than the N-terminal and C-terminal regions.

TABLE 12

| | |
|---|---|
| HP | MPSSLPGSQVPHPTLDAVDLVEKTLRNEGTSSSAPVLEEGDTDPWTLPQLKDTSQPWKEL |
| | * .. *.***. *.* *.. *.** |
| PN | MAPRQKVGTNSSPKPALDDDAPVGNIPPAYSTLDLVSDDPDADPYNAPELIDNGVKWSEL |
| HP | RVAGRLRRVAGSVLKACGLLGSLYFFICSLDVLSSAFQLLGSKVAGDIFKDNVVLSNPVA |
| | . *... ** ...** . *** ***************.*.*.********.**.**** |
| PN | DTKGKMMRVLTGLLKLVALLGLLYFFICSLDVLSSAFQLVGGKAAGDIFKDNAVLANPVA |
| HP | GLVIGVLVTALVQSSSTSSSIVVSMVAAKLLTVRVSVPIIMGVNVGTSITSTLYSMAQSG |
| | *********..***************.. **.*. .******.*.***.*.*.*.* *.* |
| PN | GLYIGVLVTVMVQSSSTSSSIVVSMVSSGLLDVQSAVPIIMGANIGTSVTNTIVAMMQAG |
| HP | DRDEFQRAFSGSAVHGIFNWLTVLVLLPLESATALLERLSELALGAASLRPRAQAPDILK |
| | **.**.***.*..**..****.**.***** **..* .*..* ... .. ..***.*. |
| PN | DRNEFRRAFAGATVHDFFNWLAVLILLPLEVATGVLYKLTHLIIESFNIQGGEDAPDLLN |
| HP | VLTKPLTHLIVQLDSDMI--MSSATGNATNSSLIKHWCGTTGQPT---QENSSCGAFGPC |
| | *.*.***. *****...* .......*.* ****.** *... * .. ..*.* . * |
| PN | VITDPLTDSIVQLKDNVISLIATNDEAAVNMSLIKEWCKTKTNVTFWNATVENCTAGALC |
| HP | TEKNSTA--------PADRLPCRHLFAGTELTDLAVGCILLAGSLLVLCGCLVLIVKLLN |
| | *... . .. .*.*.**.*.*.***** **** **.***.**.******* |
| PN | WEEGNLTWTMLNKTWIINQERCKHIFANTTLPDLAVGLILLALSLFVLCTCLILIVKLLN |
| HP | SVLRGRAQVVRTVINADFPFPLGSVLGGYLAVLAGAGLTFALQSSSVFTAAVVPLMGVGV |
| | *.*.*.** *.. ***.*****. *..**.*...***.** .*******.*..**.*.** |
| PN | SMLKGQVAVVIKRVINTDFPFPFCIVTGYIAIFVGAGMTFIVQSSSVFTSAITPLVGIGY |
| HP | ISLDRAYPLLLGSNIGTTTTALLAALASPADRMLSALQVALIHFFFNLAGILLWYLVPAL |
| | ***.***** **********.***. ****... ..**.** *****. ****.* .* |
| PN | ISLERAYPLTLGSNIGTTTTAILAAMASPAEKLKESLQIALCHFFFNVMGILLFYPIPFT |
| HP | RLPIPLARHFGVVTARYRWVAGVYLLLGFLLLPLAAFGLSLAGGMVLAAVGGPLVGLVLL |
| | *.**.*** .* **.*** **.*** **..**..****.** **..** *.* *... |
| PN | RVPIRLARGLGNHTAKYRWFAGLYLVLCFLVFPLTVFGLSMAGWQVLVGVGVPFVVLIVF |
| HP | VILVTVLQRRRPAWLPVRLRSWAWLPVWLHSLEPWDRLVTRCCPCNVCSPPKATTKEAYC |
| | **.*.*.*.* .** *..*..** ***..*** .** |
| PN | VIVVNVMQSRCPRFLPKVLQDWDFLPRPLHSMAPWDTVVTSALGFCGKYCCCCCKCCKKT |
| HP | YENPEILASQQL |
| PN | EDENMMKNNTKSLEMYDNPSMLKDEDTKEASKATHL |

The search of the GenBank using the base sequences of the present cDNA has revealed the registration of sequences that shared a homology of 90% or more (for example, Accession No. AI792826) among ESTs. However, since they are partial sequences, it can not be judged whether or not they encode the same protein as the protein of the present invention.

<HP03884> (SEQ ID NOS: 62, 72, and 82)

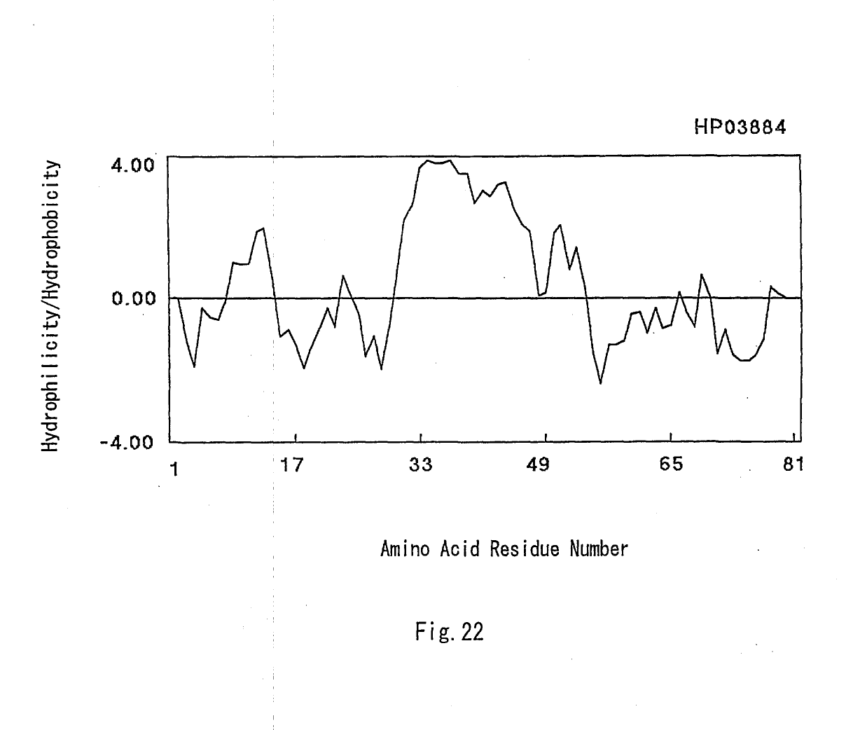

Determination of the whole base sequence of the cDNA insert of clone HP03884 obtained from cDNA library of human kidney revealed the structure consisting of a 336-bp 5′-untranslated region, a 246-bp ORF, and a 864-bp 3′-untranslated region. The ORF encodes a protein consisting of 81 amino acid residues and there existed one putative transmembrane domain. FIG. 22 depicts the hydrophobicity/hydrophilicity profile, obtained by the Kyte-Doolittle method, of the present protein. In vitro translation resulted in formation of a translation product of 10 kDa that was almost identical with the molecular weight of 8,928 predicted from the ORF.

The search of the protein database using the amino acid sequence of the present protein revealed that the protein was similar to rat cortexin (Accession No. P41237). Table 13 shows the comparison between amino acid sequences of the human protein of the present invention (HP) and rat cortexin (RC). Therein, the marks of -, *, and . represent a gap, an amino acid residue identical with that of the protein of the present invention, and an amino acid residue similar to that of the protein of the present invention, respectively. The both proteins shared a homology of 47.9% in the entire region.

TABLE 13

```
HP MDGGQPIPSSLVPLGNESADSSMSLEQKMTFVFVILLFIFLGILIV
                      *..* * .. ........**. .*.**.*.*.*.*  *.
RC MSAPWTLSPEPLPPSTGPPVGAGLDVEQRTVFAFVLCLLVVLVLLMV
HP RCFRILLDPYRSMPTSTIVABGLEGLEKGQFDHALA
      **.****.** *.. * .*.*** *******..*
RC RCVRILLDPYSRMPASSWTDHKEALERGQFDYALV
```

Furthermore, the search of the GenBank using the base sequences of the present cDNA has revealed the registration of sequences that shared a homology of 90% or more (for example, Accession No. AI791379). among ESTs. However, since they are partial sequences, it can not be judged whether or not they encode the same protein as the protein of the present invention.

<HP03934> (SEQ ID NOS: 63, 73, and 83)

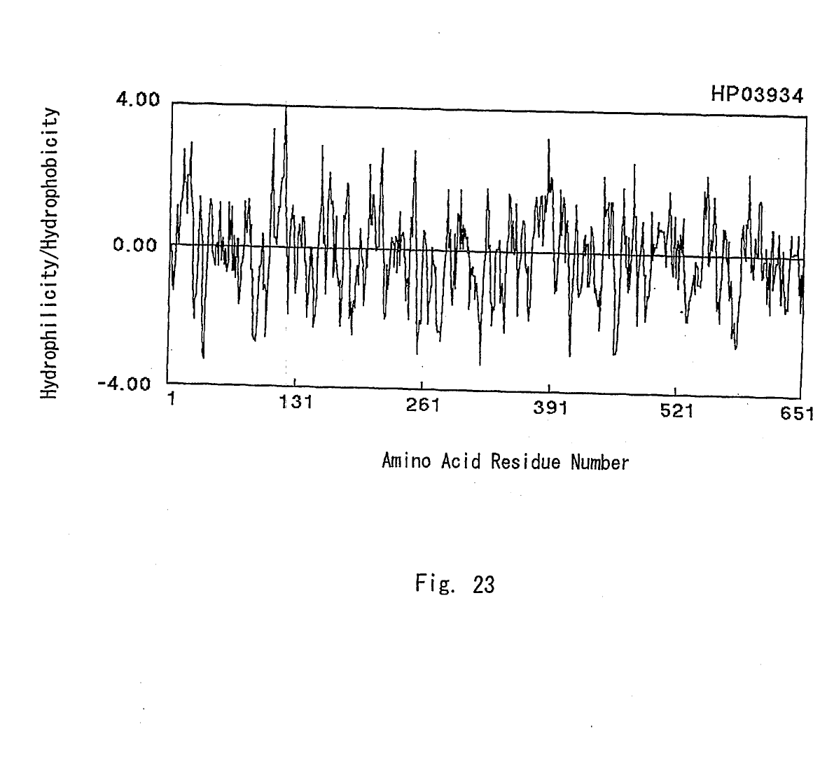

Determination of the whole base sequence of the cDNA insert of clone HP03934 obtained from cDNA library of human kidney revealed the structure consisting of a 39-bp 5′-untranslated region, a 1965-bp ORF, and a 463-bp 3′-untranslated region. The ORF encodes a protein consisting of 654 amino acid residues and there existed a putative secretory signal at the N-terminus. FIG. 23 depicts the hydrophobicity/hydrophilicity profile, obtained by the Kyte-Doolittle method, of the present protein. In vitro translation resulted in formation of a translation product of 80 kDa that was larger than the molecular weight of 74,110 predicted from the ORF. Application of the (-3,-1) rule, a method for predicting the cleavage site of the secretory signal sequence, allows to expect that the mature protein starts from arginine at position 28.

The search of the protein database using the amino acid sequence of the present protein revealed that the protein was similar to human β-galactosidase (Accession No. AAC12775). Table 14 shows the comparison between amino acid sequences of the human protein of the present invention (HP) and human β-galactosidase (BG). Therein, the marks of -, *, and . represent a gap, an amino acid residue identical with that of the protein of the present invention, and an amino acid residue similar to that of the protein of the present invention, respectively. The both proteins shared a homology of 54.6% in the entire region.

TABLE 14

| | |
|---|---|
| HP | MAPKKLSCLRSLLLPLSLTLL-----LPQADTRSFVVDRGHDRFLLDGAPFRYVSGSLHY |
| | .* *** * * ** *..*. * * .* ..*.** **.****.***.** |
| GB | MPGFLVRILLLLLVLLLLGPTRGLRNATQRMFEIDYSRDSFLKDGQPFRYISGSIHY |
| HP | FRVPRVLWADRLLKMRWSGLNAIQFYVPWNYHEPQPGVYNFNGSRDLIAFLNEAALANLL |
| | **** * ******. .****** *****.*** ** *.*....*. **. * .** |
| GB | SRVPRFYWKDRLLKMKMAGLNAIQTYVPWNFHEPWPGQYQFSEDHDVEYFLRLAHELGLL |
| HP | VILRPGPYICAEWEMGGLPSWLLRKPEIHLRTSDPDFLAAVDSWFKVLLPKIYPWLYHNG |
| | *******************.*** * .* **.****.*****.*. *****. * **.** |
| BG | VILRPGPYICAEWEMGGLPAWLLEKESILLRSSDPDYLAAVDKWLGVLLPKMKPLLYQNG |
| HP | GNIISIQVENEYGSYRACDFSYMRHLAGLFRALLGEKILLFTTDGPE--GLKCGSLRGLY |
| | * .*..********* ****.*.* *. ..** **....*****.. ****.*.*** |
| BG | GPVITVQVENEYGSYFACDFDYLRFLQKRFRHHLGDDVVLFTTDGAHKTFLKCGALQGLY |
| HP | TTVDFGPADNMTKIFTLLRKYEPHGPLVNSEYYTGWLDYWGQNHSTRSVSAVTKGLENML |
| | *****...*.*. * **.**.***.***.******.*** *** ...**...* ..* |
| BG | TTVDFGTGSNITDAFLSQRKCEPKGPLINSEFYTGWLDHWGQPHSTIKTEAVASSLYDIL |
| HP | KLGASVNMYMFHGGTNFGYWNGADKKGRFLPITTSYDYDAPISEAGDPTPKLFALRDVIS |
| | *****.*** *****.*****. ... . .********.***** * * ****..* |
| BG | ARGASVNLYMFIGGTNFAYWNGAN--SPYAAQPTSYDYDAPLSEAGDLTEKYFALRNIIQ |
| HP | KPQEVPLGPLPPPSPKMMLGPVTLHLVGHLLAFLDLLCPRGPIRSILPMTFEAVKQDHGF |
| | **..** **.**..**. * ***. . . * **.***.***.*. *.** .***..** |
| BG | KFEKVPEGPIPPSTPKFAYGKVTLEKLKTVGAALDILCPSQPIKSLYPLTFIQVKQHYGF |
| HP | MLYRTYMTHTIFEPTPFWVPNNGVHDRAYVMVDGVFQGVVERNMRDKLFLTGKLGSKLDI |
| | .**** .... .*.*. * ********* ***. ***.*** .* .*** *..**. |
| BG | VLYRTTLPQDCSNPAPLSSPLNGVHDRAYYAVDGIPQGVLERNNYITLNITGKAGATLDL |
| HP | LVENMGRLSFGSNSSDFKGLLKPPILGQTILTQWMMFPLKIDNLVK-----W--W-FPLQ |
| | *******...*. .*****.. .*. .***.* .***.... *. * . . |
| BG | LVENMGRVNYGAYINDFKGLVSNLTLSSNILTDWTIFPLDTEDAVRSKHGGWGHRDSGHH |
| HG | LPKWPYPQAP-SGPTFYSKTFPILGSVGD-----TFLYLPGTKGQVWINGFNLGRYWTKQ |
| | *.. . . *.** .*.* ... * **. .******************. . |
| BG | DEAWAHNSSNYTLPAFYMGNFSIPSGIPDLPQDTFIQFPGWTKGQVWINGFNLGRYWPAR |
| HP | GPQQTLYVPRFLLFPRGALNKITLLELE------DVPLQPQVQFLDKPILNSTSTLHRTH |
| | *** **.**. .*..... *.**.**** * * .* *.*.*...*. * .. |
| BG | GPQLTLFVPQHILMTSAP0NTITVLELEWAPCSSDDPELCAVTFVDRPVIGSSVTYDHPS |
| HP | INSLSADTLSASBPMELSGH |
| BG | KPVEKRLMPPPPQKNKDSWLDHV |

The search of the GenBank using the base sequences of the present cDNA has revealed the registration of sequences that shared a homology of 90% or more (for example, Accession No. AI907720) among ESTs. However, since they are partial sequences, it can not be judged whether or not they encode the same protein as the protein of the present invention.

<HP03949> (SEQ ID NOS: 64, 74, and 84)

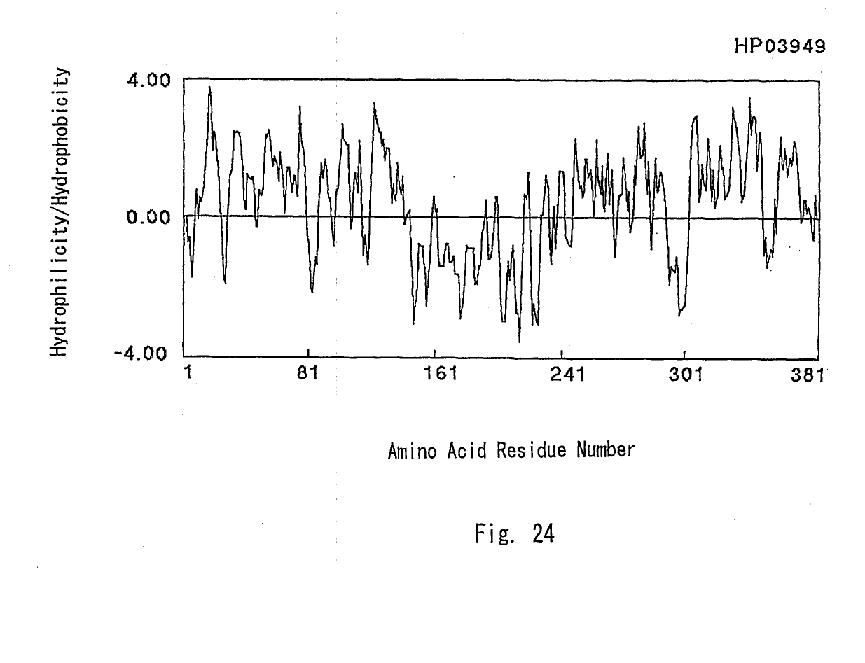

Determination of the whole base sequence of the cDNA insert of clone HP03949 obtained from cDNA library of human kidney revealed the structure consisting of a 244-bp 5′-untranslated region, a 1173-bp ORF, and a 33-bp 3′-untranslated region. The ORF encodes a protein consisting of 390 amino acid residues and there existed ten putative transmembrane domains. FIG. 24 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to human solute carrier family 16 (Accession No. NM_{—}004696). Table 15 shows the comparison between amino acid sequences of the human protein of the present invention (HP) and human solute carrier family 16 (HS). Therein, the marks of -, *, and . represent a gap, an amino acid residue identical with that of the protein of the present invention, and an amino acid residue similar to that of the protein of the present invention, respectively. The both proteins shared a homology of 98.7% in the region other than the N-terminal and C-terminal regions.

TABLE 15

| | |
|---|---|
| HP | MGMDDCDSFFPGPLVAIICDILGEKTTSILGAFVVTGGYLISSWATSIPFLCVTMGLL |
| | * .*********************************************** |
| HS | WIGSIMSSLRFCAGPLVAIICDILGEKTTSILGAFVVTGGYLISSWATSIPFLCVTMGLL |
| HP | PGLGSIWLYQVAAVVTTKYFKKRLAISTAIARSGMGLTFLLAPFTKFLIDLYDWTGAIIL |
| | ************************************************************ |
| HS | PGLGSAFLYQYAAVVTTKYFKKRLALSTALARSGMGLTFLLAPFTKFLIDLYDWTGALIL |
| HP | FGAIALNLVPSSMLLRPIHIKSENNSGIKDKGSSLSAHGPEAHATETHCHETEESTIKDS |
| | ************************************************************ |
| HS | FGAIALNLVPSSMLLRPIHIKSENNSGIKDKGSSLSAHGPEAHATETHCHETEESTIKDS |
| HP | TTQKAGLPSKNLTVSQNQSEEFYNGPNRNRILLKSDBESDKVISWSCKQLFDISLFRNPF |
| | ************************************************************ |
| HS | TTQKAGLPSKNLTVSQNQSEEFYNGPNRNRLLLKSDEESDKVISWSCKQLFDISLFRNPF |
| HP | FYIFTWSFLLSQLAYFIPTFHLVARAKTLGIDIMDASYLVSVAGILETVSQIISGWVADQ |
| | ************************************************************ |
| HS | FYIFTWSFLLSQLAYFIPTFHLVARAKTLGIDIMDASYLVSVAGILETVSQIISGWVADQ |
| HP | NWIKKYHYHKSYLILCGITNLLAPLATTFPLLMTYTICFAIFAGGYLALILPVLVDLCRN |
| | ************************************************************ |
| HS | NWIKKYHYHKSYLILCGITNLLAPLATTFPLLMTYTICFAIFAGGYLALILPVLVDLCRN |
| HP | STVNRFLGLASFFAGMAVLSGPPIAGNTFTTF |
| | ************************** . |
| HS | STVNRFLGLASFFAGMAVLSGPPIAGWLYDYTQTYNGSFYFSGICYLLSSVSFFFVPLAE |

The search of the GenBank using the base sequences of the present cDNA has revealed the registration of sequences that shared a homology of 90% or more (for example, Accession No. AW239415) among ESTs. However, since they are partial sequences, it can not be judged whether or not they encode the same protein as the protein of the present invention.

<HP03959> (SEQ ID NOS: 65, 75, and 85)

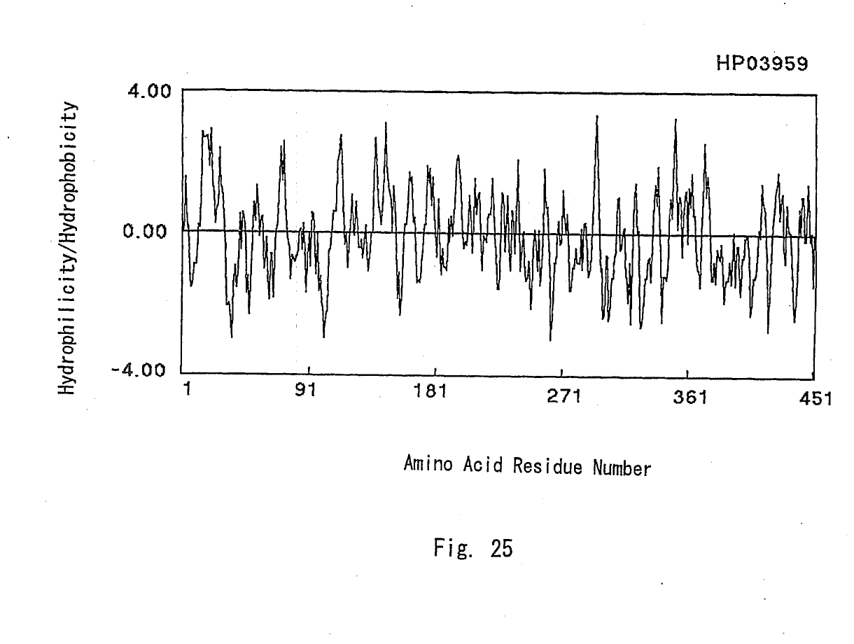

Determination of the whole base sequence of the cDNA insert of clone HP03959 obtained from cDNA library of human kidney revealed the structure consisting of a 7-bp 5′-untranslated region, a 1359-bp ORF, and a 531-bp 3′-untranslated region. The ORF encodes a protein consisting of 452 amino acid residues and there existed a putative secretory signal at the N-terminus. FIG. 25 depicts the hydrophobicity/hydrophilicity profile, obtained by the Kyte-Doolittle method, of the present protein. In vitro translation resulted in formation of a translation product of 53 kDa that was somewhat larger than the molecular weight of 50,798 predicted from the ORF. In this case, the addition of a microsome led to the formation of a product of 55 kDa. In addition, there exists in the amino acid sequence of this protein three sites at which N-glycosylation may occur (Asn-Phe-Ser at position 64, Asn-Gly-Ser at position 126 and Asn-Val-Thr at position 362). Application of the (-3,-1) rule, a method for predicting the cleavage site of the secretory signal sequence, allows to expect that the mature protein starts from alanine at position 27.

The search of the protein database using the amino acid sequence of the present protein revealed that the protein was similar to Arabidopsis thaliana putative carboxypeptidase (Accession No. AAD21510). Table 16 shows the comparison between amino acid sequences of the human protein of the present invention (HP) and Arabidopsis thaliana putative carboxypeptidase (AC). Therein, the marks of -, *, and . represent a gap, an amino acid residue identical with that of the protein of the present invention, and an amino acid residue similar to that of the protein of the present invention, respectively. The both proteins shared a homology of 44.3% in the region of 323 amino acid residues other than the N-terminal and C-terminal regions.

TABLE 16

| | |
|---|---|
| HP | MELALRRSPVPRWLLLLPLLLGLNAGAVIDWPTEEGKEVWDYVTVRKDAYMFWWLYYATN |
| AC | MDPKLGDTSKLDQHTCFGGIIKV |
| HP | SCKNFSELPLVMWLQGGPGGSSTGFGNFEEIGPLDSDLKPRKTTWLQAASLLFVDNPVGT |
| | *. *.*..*.***.*.****. ****..***. *.*****.***. |
| AC | HIELKILPSHGLSSSGSKGASGVGIGNFQEVGPLDTFLKPRNSTWLKKADLLFVDSPVGA |
| HP | GFSYVNGS--GAYAKDLAMVASDMMVLLKTFFSCHKEFQTVPFYIFSESYGGKMAGGIGL |
| | *.*.*.*. . *.*. . .* *. **. .*. ..... *..* .******.*. .** |
| AC | GYSFVEGNQKDLYVKSDEEAAQDLTKLLQQLFNKNQTLNQSPLFIVAESYGGKIAVKLGL |
| HP | ELYKAIQRGTIKCNFAGVALGDSWISPVDSVLSWGPYLYSMSLLEDKGLAEVSKVAEQVL |
| | .. .*.*.*..* ...** ******** * *.**** * .* *.*.**.. ...**.. |
| AC | SVIDAVQSGKLKLHLGGVILGDSWISPEDFVFSWCPLLKHVSRLDDNGLDSSNSLAEKIK |
| HP | NAVNKGLYREATELWGKAEMIIEGNTDGVNFYN-ILTKSTPTSTMESSLEFTQSHLV--- |
| | .....* * .**. * . * .*. ... *.***.*... . ....**...... . |
| AC | TQIKNGEYVGATQTWMDLENLISSKSNFVDFYNFLLDTGMDPVSLTTSLKIKKEEKIKKY |
| HP | __CLCQ-RHVRHLQR--DALSQLMNGPIRKKLKIIPEDQSWGGQATNVFVNMEEDFMKPV |
| | .* . * ..... ..*..**** *.*******.* **.....**.**..***** |
| AC | SRYLNDMRSLSDVEDVEGDLDKLMNGVIKKKLKIIPNDLIWGNNSDDVFTAMEAAFMKPV |
| HP | ISIVDELLEAGINVTVYNGQLDLIVDTMGQEAWVRKLKWPELPKFSQLKWKALYSDPKSL |
| | *. *****..*..**.******.* .* * ****.**. |
| AC | IEDVDELLATGVDVTIYNGQLDVICSTSGTEAWVHKLR |

The search of the GenBank using the base sequences of the present cDNA has revealed the registration of sequences that shared a homology of 90% or more (for example, Accession No. T59065) among ESTs. However, since they are partial sequences, it can not be judged whether or not they encode the same protein as the protein of the present invention.

<HP03983> (SEQ ID NOS: 66, 76, and 86)

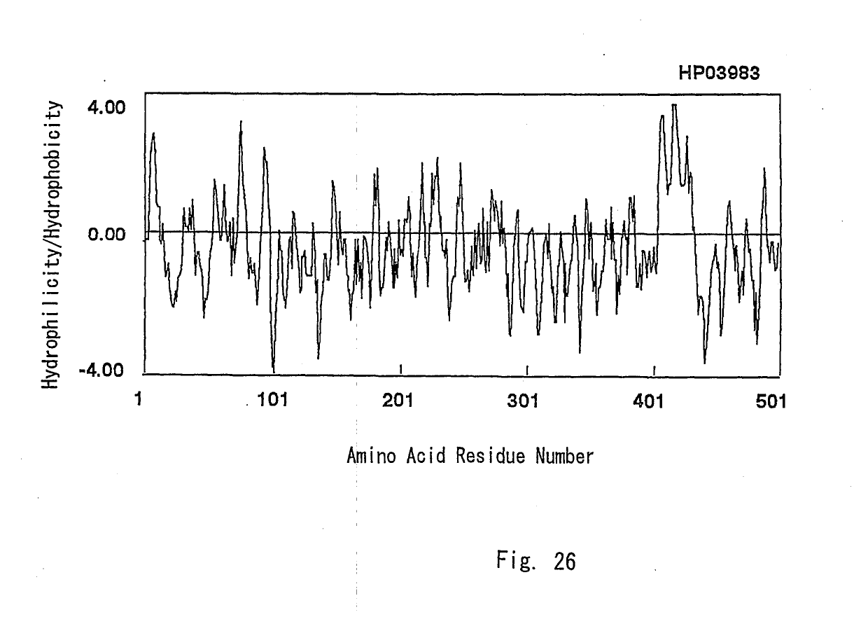

Determination of the whole base sequence of the cDNA insert of clone HP03983 obtained from cDNA library of human kidney revealed the structure consisting of a 42-bp 5′-untranslated region, a 1473-bp ORF, and a 341-bp 3′-untranslated region. The ORF encodes a protein consisting of 490 amino acid residues and there existed a putative secretory signal at the N-terminus and one putative transmembrane domain at the C-terminus. FIG. 26 depicts the hydrophobicity/hydrophilicity profile, obtained by the Kyte-Doolittle method, of the present protein. Application of the (-3,-1) rule, a method for predicting the cleavage site of the secretory signal sequence, allows to expect that the mature protein starts from glutamic acid at position 22.

The search of the protein database using the amino acid sequence of the present protein revealed that the protein was similar to human C1qR protein (Accession No. AAB53110). Table 17 shows the comparison between amino acid sequences of the human protein of the present invention (HP) and human C1qR protein (HC). Therein, the marks of -, *, and . represent a gap, an amino acid residue identical with that of the protein of the present invention, and an amino acid residue similar to that of the protein of the present invention, respectively. The both proteins shared a homology of 25.8% in the N-terminal region of 310 amino acid residues. Since the positions of 17 cysteine residues are conserved, in particular, the two proteins are considered to assume similar higher-order structures.

TABLE 17

| | |
|---|---|
| HP | MRPAFALCLLWQALWPGPGGGEHPTADRAGCSASGACYSLHHATMKRQAAEEACILRGGA |
| | * ....* ** * . **.*. .... . * ...***. * .... ..*.. * .**. |
| HC | MATSMGLLLLLLLLLTQPGAGTGADTEAVVC-VGTACYTAHSGKLSAAEAQNHCNQNGGN |
| HP | LSTRAGAELRAVLALL---RAGPGPGGGSKDLLFWVALERRRSHCTLENEPLRGFSWLSS |
| | *.**.. .* . * .* .... ... **..*.* ...* . **.****. |
| HC | LATVKSKEEAQHVQRVLAQLLRREAALTARMSKFWIGLQREKGKCLDPSLPLKGFSWV-- |
| HP | DPGGLESDTLQWVEEPQRSCTARRC--AVLQATGGVEP---AGWKEMRC------HLRAN |
| | ** .. .* .* ..**...** .*. . . * . *.* .* . . |
| HC | -GGGEDTPYSNWHKELRNSCISKRCVSLLLDLSQPLLPNRLPKWSEQPCGSPGSPGSNIE |
| HP | GYLCKYQPBVLCPAPRPGAASNLSYRAFFQLHSAALDFSPPGTEVSALC----RGQLPIS |
| | *..**. *. .*.. *......* .*** *..*. * ...... * ... . |
| HC | GFVCKFSFKGMCRPLALGGPGQVTYTTPFQTTSSSLEAVPFASAANVACGEGDKDETQSH |
| HP | -VTCIADEIGA-RWDKLSGDVLCPCP--GRYLRAGKCAELPNCLD-DLGGFACECATGFE |
| | * ... .. *. *.. ** .* * ...* * . .*.. . *.* *.* .** |
| HC | YFLCKEKAPDVFDWG--SSGPLCVSPKYGCNFNNGGCHQ--DCFEGGDGSFLCGCRPGFR |
| HP | LGKDGRSCVTSGEGQPTLGGTGVPTRRPPATATSPVPQRTWPIRVDEKLGETPLVPEQDN |
| | * .* .*.. |
| HC | LLDDLVTCASRNPCSSSPCRGGATCVLGPHGKNYTCRCPQGYQLDSSQLDCVDVDECQDS |
| HP | SVTSIPEIPRWGSQSTMSTLQMSLQAESKATITPSGSVISKFNSTTSSATPQAFDSSSAV |
| HC | PCAQECVNTPGGFRCECYVGYEPGGPGEGACQ1WDECALGRSPCAQGCTNTDGSFHCSCE |
| HP | VPIFVSTAVVYLVILTMTVLGLVKLCFHESPSSQPRKESMGPPGLESDPEPAALGSSSAH |
| HC | BGYVLAGEDGTQCQDVDECVGPGGPLCDSLCFNTQGSFHCGCLPGWVLAPNGVSCTMGPV |
| HP | CTNNGVKYGDCDLRDRAEGALLAESPLGSSDA |
| HC | SLGPPSGPPDEEDKGEKEGSTVPRAATASPTRGPEGTPKATPTTSRPSLSSDAPITSAPL |

The search of the GenBank using the base sequences of the present cDNA has revealed the registration of sequences that shared a homology of 90% or more (for example, Accession No. R51653) among ESTs. However, since they are partial sequences, it can not be judged whether or not they encode the same protein as the protein of the present invention.

<HP10745> (SEQ ID NOS: 67, 77, and 87)

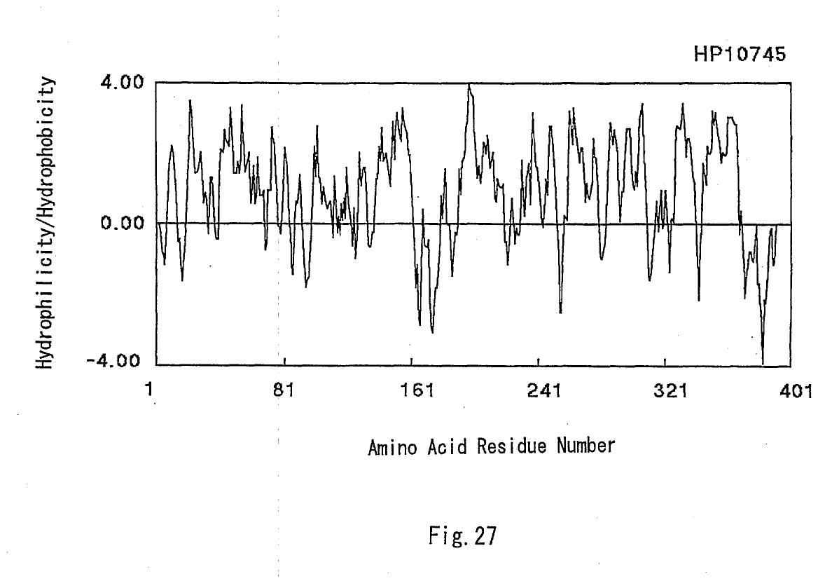

Determination of the whole base sequence of the cDNA insert of clone HP10745 obtained from cDNA library of human umbilical cord blood revealed the structure consisting of a 261-bp 5′-untranslated region, a 1179-bp ORF, and a 733-bp 3′-untranslated region. The ORF encodes a protein consisting of 392 amino acid residues and there existed nine putative transmembrane domains. FIG. 27 depicts the hydrophobicity/hydrophilicity profile, obtained by the Kyte-Doolittle method, of the present protein.

The search of the GenBank using the base sequences of the present cDNA has revealed the registration of sequences that shared a homology of 90% or more (for example, Accession No. R59881) among ESTs. However, since they are partial sequences, it can not be judged whether or not they encode the same protein as the protein of the present invention.

<HP10775> (SEQ ID NOS: 68, 78, and 88)

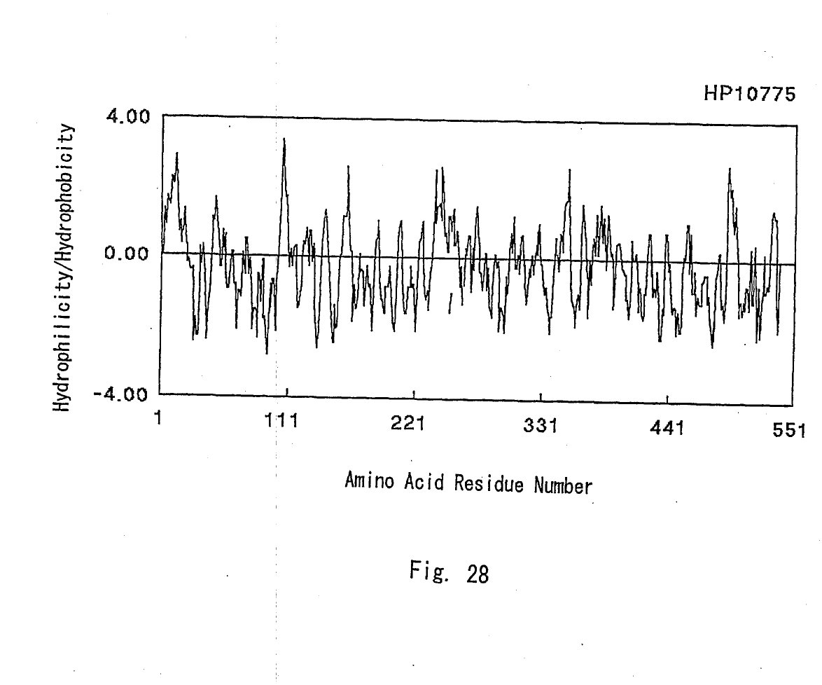

Determination of the whole base sequence of the cDNA insert of clone HP10775 obtained from cDNA library of human kidney revealed the structure consisting of a 30-bp 5′-untranslated region, a 1617-bp ORF, and a 287-bp 3′-untranslated region. The ORF encodes a protein consisting of 538 amino acid residues and there existed a putative secretory signal at the N-terminus. FIG. 28 depicts the hydrophobicity/hydrophilicity profile, obtained by the Kyte-Doolittle method, of the present protein. In vitro translation resulted in formation of a translation product of 66 kDa that was larger than the molecular weight of 55,133 predicted from the ORF. Application of the (-3,-1) rule, a method for predicting the cleavage site of the secretory signal sequence, allows to expect that the mature protein starts from serine at position 23.

The search of the GenBank using the base sequences of the present cDNA has revealed the registration of sequences that shared a homology of 90% or more (for example, Accession No. AA366320) among ESTs. However, since they are partial sequences, it can not be judged whether or not they encode the same protein as the protein of the present invention.

<HP10782> (SEQ ID NOS: 69, 79, and 89)

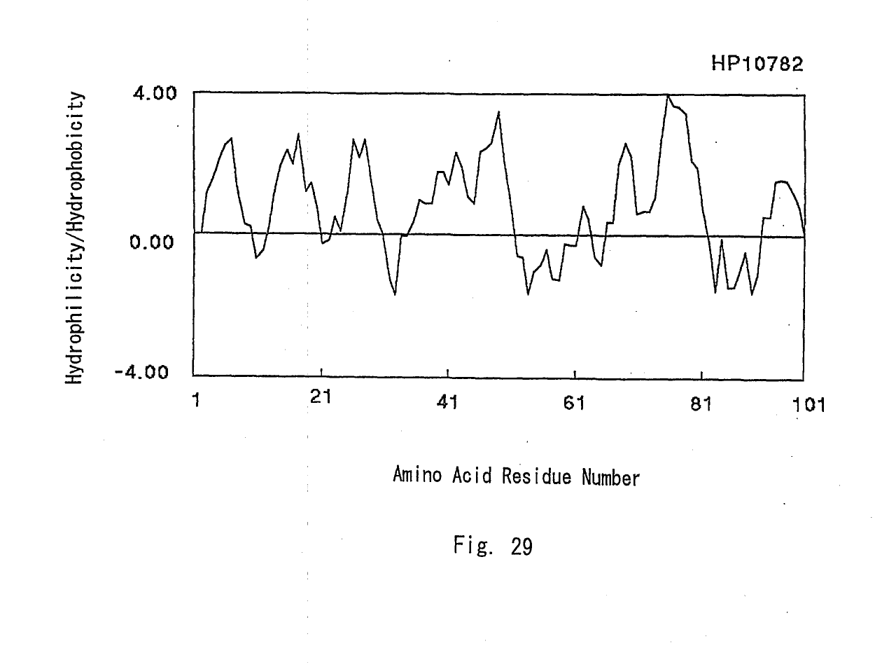

Determination of the whole base sequence of the cDNA insert of clone HP10782 obtained from cDNA library of human kidney revealed the structure consisting of a 70-bp 5′-untranslated region, a 309-bp ORF, and a 1501-bp 3′-untranslated region. The ORF encodes a protein consisting of 102 amino acid residues and there existed three putative transmembrane domains. FIG. 29 depicts the hydrophobicity/hydrophilicity profile, obtained by the Kyte-Doolittle method, of the present protein.

The search of the GenBank using the base sequences of the present cDNA has revealed the registration of sequences that shared a homology of 90% or more (for example, Accession No. AI815463) among ESTs. However, since they are partial sequences, it can not be judged whether or not they encode the same protein as the protein of the present invention.

<HP10787> (SEQ ID NOS: 70, 80, and 90)

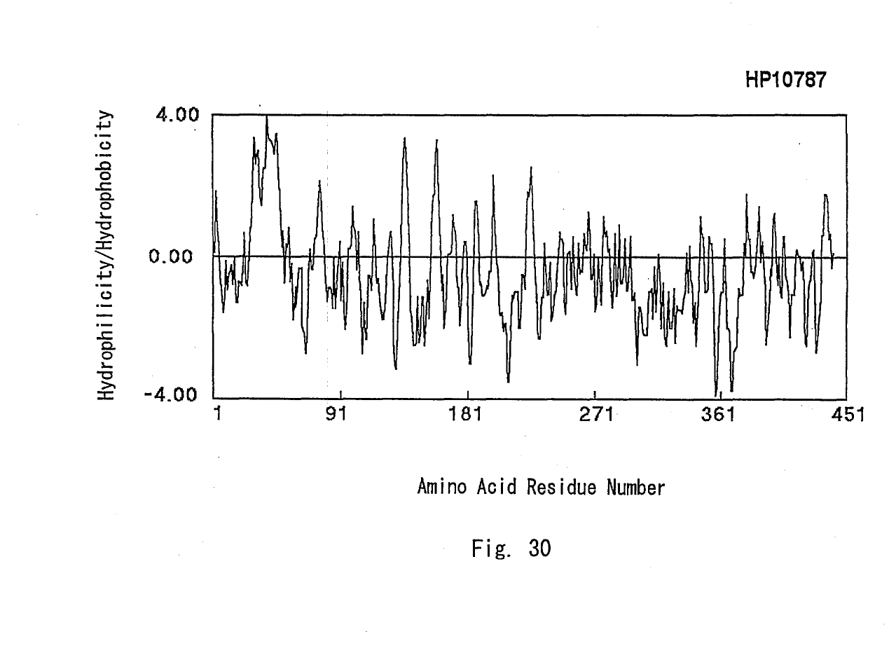

Determination of the whole base sequence of the cDNA insert of clone HP10787 obtained from cDNA library of human kidney revealed the structure consisting of a 54-bp 5′-untranslated region, a 1329-bp ORF, and a 912-bp 3′-untranslated region. The ORF encodes a protein consisting of 442 amino acid residues and there existed one putative transmembrane domain at the N-terminus. FIG. 30 depicts the hydrophobicity/hydrophilicity profile, obtained by the Kyte-Doolittle method, of the present protein. In vitro translation resulted in formation of a translation product of 50 kDa that was almost identical with the molecular weight of 50,562 predicted from the ORF. In this case, the addition of a microsome led to the formation of a product of 56 kDa. In addition, there exists in the amino acid sequence of this protein four sites at which N-glycosylation may occur (Asn-Leu-Thr at position 83, Asn-Phe-Thr at position 89, Asn-Ala-Ser at position 113 and Asn-Lys-Ser at position 151).

The search of the protein database using the amino acid sequence of the present protein revealed that the protein was similar to rat PV-1 (Accession No. AAD41524). Table 18 shows the comparison between amino acid sequences of the human protein of the present invention (HP) and rat PV-1 (RP). Therein, the marks of -, *, and . represent a gap, an amino acid residue identical with that of the protein of the present invention, and an amino acid residue similar to that of the protein of the present invention, respectively. The both proteins shared a homology of 61.1% in the entire region.

TABLE 18

| | |
|---|---|
| HP | MGLAMEHGGSYARAGGSSRGCWYYLRYFFLFVSLIQFLIILGLVLFMVYGNVHVSTESNL |
| | ***.*.. ..*.*.*...*****************************.*****..***.* |
| RP | MGLSMDR-SPYSRTGDRDRGCWYYLRYFFLFVSLIQFLIILGLVLFMIYGNVHATTESSL |
| HP | QATERRAEGLYSQLLGLTASQSNLTKELNFTTRAKDAIMQMWLNARRDLDRINASFRQCQ |
| | .*** **..****..**.*.*.**.*.**..* .**..** *..**...********** |
| RP | RATEIRADNLYSQVVGLSAAQANLSKQLNISTLVKDTVMQQLLTTRREVERINASFRQCQ |
| HP | GDRVIYTNNQRYMAAIILSEKQCRDQFKDMNKSCDALLFMLNQKVKTLEVEIAKEKTICT |
| | ** ..*.* .*..**********..*.*. **.*.****.*..******.*..***..*. |
| RP | GDLITYINYNRFIAAIILSEKQCQEQLKEGNKTCEALLFKLGEKVKTLEMEVVKEKAVCS |
| HP | KDKBSVLLNKRVAEEQLVECVKTRELQHQERQLAKEQLQKVQALCLPLDKDKFEMDLRNL |
| | ***.*.* .** ** * .* *.** *.*..*...***.***.******..**. *. *. |
| RP | KDKDSLLAGKRQAEMQQEACGKAREQQKQDQQVTEEQLRKVQSLCLPLDQEKFQADVLNY |
| HP | WRDSIIPRSLDNLGYNLYHPLGSELASIRRACDHMPSLMSSKVEELARSLRADIERVARE |
| | ****.. *****.**. * .* .*..*.**.*. .*..*..*******.***.****.** |
| RP | WRDSLVYRSLDNIGYN-Y-SLMPEFSSLRRTCESLPGIMTTKVEELARGLRAGIERVTRE |
| HP | NSDLQRQKLEAQQGLRASQEAKQKVEKEAQAREAKLQAECSRQTQLALEEKAVLRKERDN |
| | *..*.***** ........**. ....******..*..**.***********.**..**. |
| RP | NGELRRQKLELERAIQGEREARTRAGTEAQARETQLRTECARQTQLALEEKAALRTQRDD |
| HP | LAKELEEKKREAEQLRMELAIRNSALDTGIKTKSQPMMPVSRPMGPVPNPQPIDPASLEE |
| | *...**..*** **** *...* ******.*.** * .. .* ** ***.********* |
| RP | LERQLEARKRELEQLRTEVDVRISALDTCVKAKSLPAIQ-PRLPGPPPNPPPIDPASLEE |
| HP | FKRKILESQRPPAGLPVAPSSG |
| | **..******** *..*.** |
| RP | FKKRILESQRPPLVNPAVPPSG |

Furthermore, the search of the GenBank using the base sequences of the present cDNA has revealed the registration of sequences that shared a homology of 90% or more (for example, Accession No. AL041217) among ESTs. However, since they are partial sequences, it can not be judged whether or not they encode the same protein as the protein of the present invention.

<HP03977> (SEQ ID NOS: 91, 101, and 111)

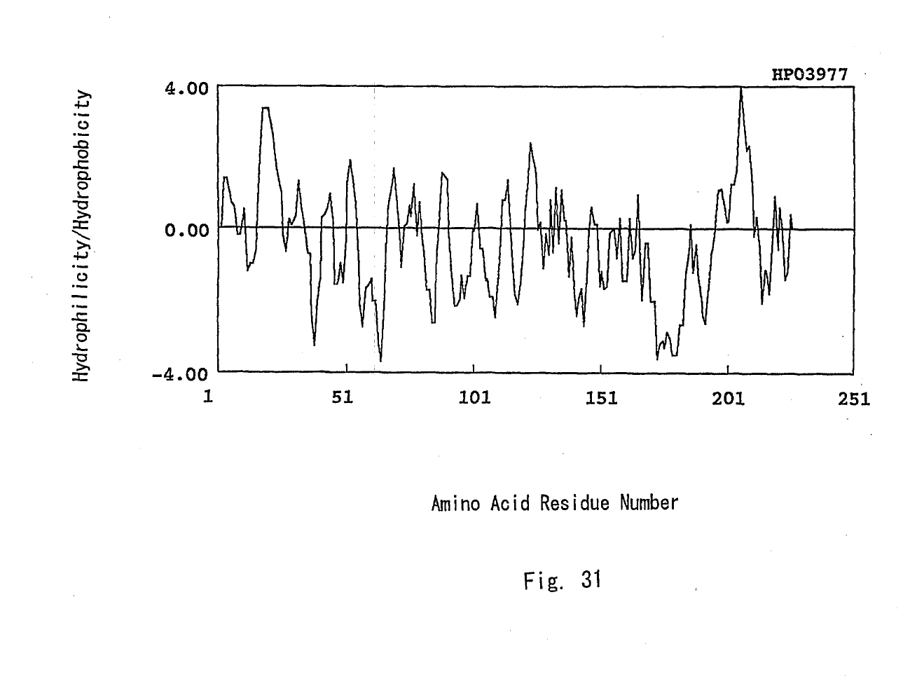

Determination of the whole base sequence of the cDNA insert of clone HP03977 obtained from cDNA library of human kidney revealed the structure consisting of a 35-bp 5′-untranslated region, a 684-bp ORF, and a 1175-bp 3′-untranslated region. The ORF encodes a protein consisting of 227 amino acid residues and there existed a putative secretory signal at the N-terminus and one putative transmembrane domain at the C-terminus. FIG. 31 depicts the hydrophobicity/hydrophilicity profile, obtained by the Kyte-Doolittle method, of the present protein. In vitro translation resulted in formation of a translation product of 29 kDa that was larger than the molecular weight of 25,926 predicted from the ORF. Application of the (-3,-1) rule, a method for predicting the cleavage site of the secretory signal sequence, allows to expect that the mature protein starts from leucine at position 30.

The search of the protein database using the amino acid sequence of the present protein revealed that the protein was similar to human gp25L2 (Accession No. CAA62380). Table 19 shows the comparison between amino acid sequences of the human protein of the present invention (HP) and human gp25L2 (GP). Therein, the marks of -, *, and . represent a gap, an amino acid residue identical with that of the protein of the present invention, and an amino acid residue similar to that of the protein of the present invention, respectively. The both proteins shared a homology of 78.5% in the region other than the N-terminal region.

TABLE 19

| | |
|---|---|
| HP | MAGVGAGPLRAMGRQALLLLALCATGAQGLYFHIGETEKRCFIEEIPDETMVIGNYRTQM |
| | * **. * * . *. .**********.*******************. |
| GP | MRTLLLVLWLATRGS-ALYFHIGETEKKCFIEEIPDETMVIGNYRTQL |
| HP | WDKQKEVFLPSTPGLGMHVEVKDPDGKVVLSRQYGSEGRFTFTSHTPGDHQICLHSNSTR |
| | .***.* . *.***.** ******..**.*.*.***************.**********. |
| GP | YDKQREEYQPATPGFGMCVEVKDPEDKVILAREYGSEGRFTFTSHTPGEHQICLHSNSTK |
| HP | MALFAGGKLRVHLDIQVGEHANNYPEIAAKDKLTELQLRARQLLDQVEQIQKEQDYQRYR |
| | ..*****.**************.*.**.*****.*****.***..*********.***.* |
| GP | FSLFAGGMLRVHLDIQVGEHANDYAEIPAKDKLSELQLRVRQLVEQVEQIQKEQNYQRWR |
| HP | EERFRLTSESTNQRVLWWSIAQTVILILTGIWQMRHLKSFFEAKKLV |
| | ***** ************** **.**. .*.**************** |
| GP | EERFRQTSESTNQRVLWWSILQTLILVAIGVWQMRHLKSFFEAKKLV |

The search of the GenBank using the base sequences of the present cDNA has revealed the registration of sequences that shared a homology of 90% or more (for example Accession No. AR052481, U.S. Pat. No. 5,831,052) in patent data. However, since they are partial sequences, it can not be judged whether or not they encode the same protein as the protein of the present invention.

<HP10649> (SEQ ID NOS: 92, 102, and 112)

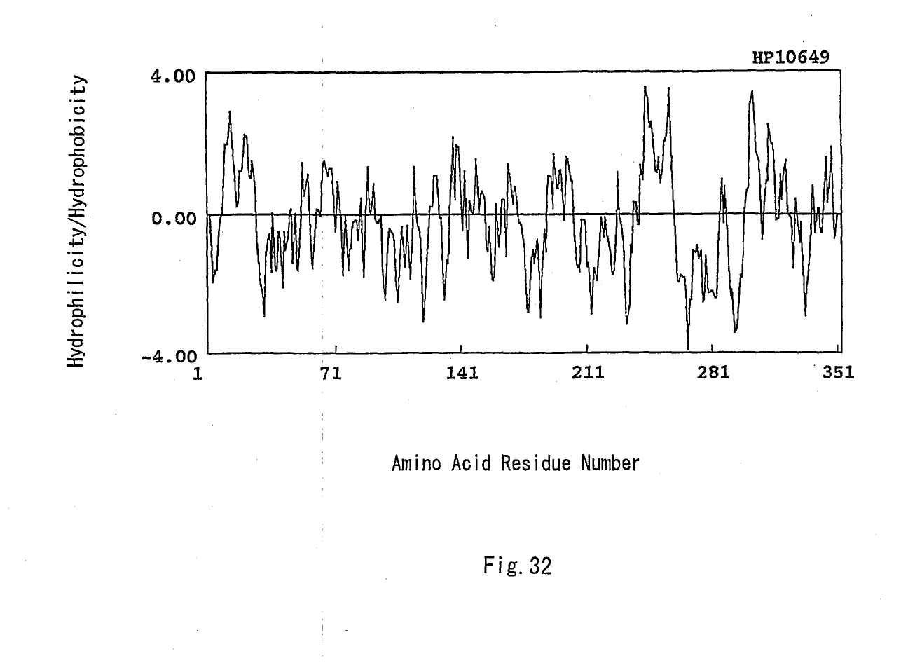

Determination of the whole base sequence of the cDNA insert of clone HP10649 obtained from cDNA library of human epidermoid carcinoma cell line KB revealed the structure consisting of a 114-bp 5′-untranslated region, a 1059-bp ORF, and a 1240-bp 3′-untranslated region. The ORF encodes a protein consisting of 352 amino acid residues and there existed one putative transmembrane domain. FIG. 32 depicts the hydrophobicity/hydrophilicity profile, obtained by the Kyte-Doolittle method, of the present protein. In vitro translation resulted in formation of a translation product of 40 kDa that was almost identical with the molecular weight of 39,774 predicted from the ORF.

The search of the protein database using the amino acid sequence of the present protein revealed that the protein was similar to Epiphyas postvittana nucleopolyhedrovirus apoptosis inhibitor iap-1 (Accession No. AAD19698). Table 20 shows the comparison between amino acid sequences of the human protein of the present invention (HP) and Epiphyas postvittana nucleopolyhedrovirus apoptosis inhibitor iap-1 (EP). Therein, the marks of -, *, and . represent a gap, an amino acid residue identical with that of the protein of the present invention, and an amino acid residue similar to that of the protein of the present invention, respectively. The both proteins shared a homology of 40.8% in the C-terminal region of 49 amino acid residues.

TABLE 20

| | |
|---|---|
| HP | MESGGRPSLCQFILLGTTSVVTAALYSVYRQKARVSQELKGAKKVHLGEDLKSILSEAPG |
| HP | KCVPYAVIEGAVRSVKETLNSQFVENCKGVIQRLTLQEHKMVWNRTTHLWNDCSKIIHQR |
| EP | MSATSPLYIINVCENAHEVSAEHVFNYLIERHNSFENYPIDNVAFVNSLIINGF |
| HP | TNTVPFDLVPHBDGVDVAVRVLKPLDSVDLGLETVYEKFHPSIQSFTDVIGHYISQERPK |
| EP | RYQNVDDAVMCEYCSAVIKNWHEDDCVEFVHATLSPYCVYANKIAQNENFANNLSTNAFL |
| HP | GIQETEEMLKVGATLTGVGELVLDNNSVRLQPPKQGMQYYLSSQDFDSLLQRQESSVRLW |
| EP | VTPGKPICVYSRLTHTNAEKSTFEDYWPAALQHLVANISEAGMFHTKLGDETACFFCDCR |
| HP | KVLALVFGFATCATLFFILRKQYLQRQERLRLKQMQEEFQEHEAQLLSRAKPEDRESLKS |
| EP | VRDWLPNDDPWQRHAIANPQCYFVVCIKGDEFCNAVRQRDELAPLQSVVALEHVSNDENM |
| HP | ACVVCLSSFKSCVFLECGHVCSCTECYRALPEPKKCPICRQAITRVIPLYNS |
| | * .**.. .. *.* * * * * .** ** .***.***..* . .. |
| EP | ECKICLERQRDTVLLPCRHFCVCMQCYFAL--DNKCPTCRQDVTDFVKIFVV |

Furthermore, the search of the GenBank using the base sequences of the present cDNA has revealed the registration of sequences that shared a homology of 90% or more (for example, Accession No. T50032) among ESTs. However, since they are partial sequences, it can not be judged whether or not they encode the same protein as the protein of the present invention.

<HP10779> (SEQ ID NOS: 93, 103, and 113)

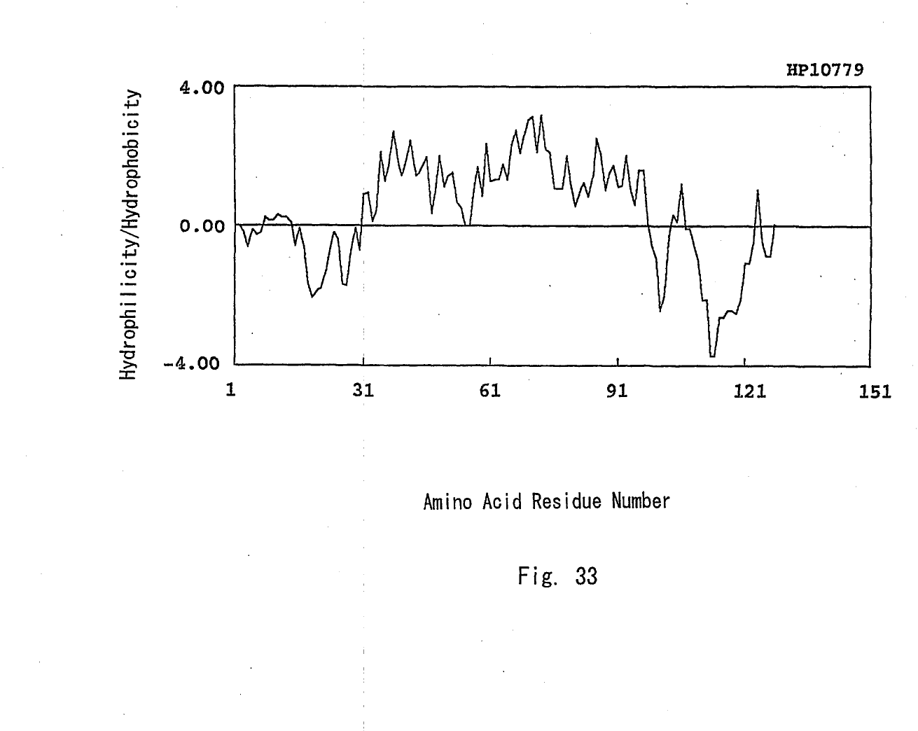

Determination of the whole base sequence of the cDNA insert of clone HP10779 obtained from cDNA library of human kidney revealed the structure consisting of a 34-bp 5′-untranslated region, a 393-bp ORF, and a 1949-bp 3′-untranslated region. The ORF encodes a protein consisting of 130 amino acid residues and there existed two putative transmembrane domains. FIG. 33 depicts the hydrophobicity/hydrophilicity profile, obtained by the Kyte-Doolittle method, of the present protein.

Furthermore, the search of the GenBank using the base sequences of the present cDNA has revealed the registration of sequences that shared a homology of 90% or more (for example, Accession No. AL042495) among ESTs. However, since they are partial sequences, it can not be judged whether or not they encode the same protein as the protein of the present invention. In addition, this gene was mapped on chromosome 9q34 (Accession No. AC001644).

<HP10790> (SEQ ID NOS: 94, 104, and 114)

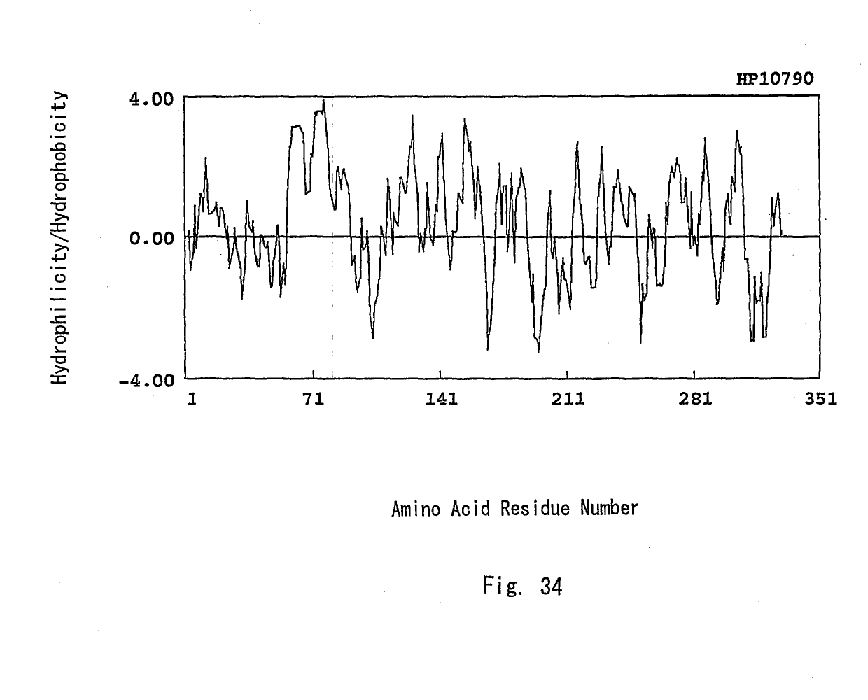

Determination of the whole base sequence of the cDNA insert of clone HP10790 obtained from cDNA library of human kidney revealed the structure consisting of a 109-bp 5′-untranslated region, a 993-bp ORF, and a 53-bp 3′-untranslated region. The ORF encodes a protein consisting of 330 amino acid residues and there existed one putative transmembrane domain. FIG. 34 depicts the hydrophobicity/hydrophilicity profile, obtained by the Kyte-Doolittle method, of the present protein. In vitro translation resulted in formation of a translation product of 34 kDa that was smaller than the molecular weight of 36,642 predicted from the ORF.

The search of the GenBank using the base sequences of the present cDNA has revealed the registration of sequences that shared a homology of 90% or more (for example, Accession No. AW241940) among ESTs. However, since they are partial sequences, it can not be judged whether or not they encode the same protein as the protein of the present invention.

<HP10793> (SEQ ID NOS: 95, 105, and 115)

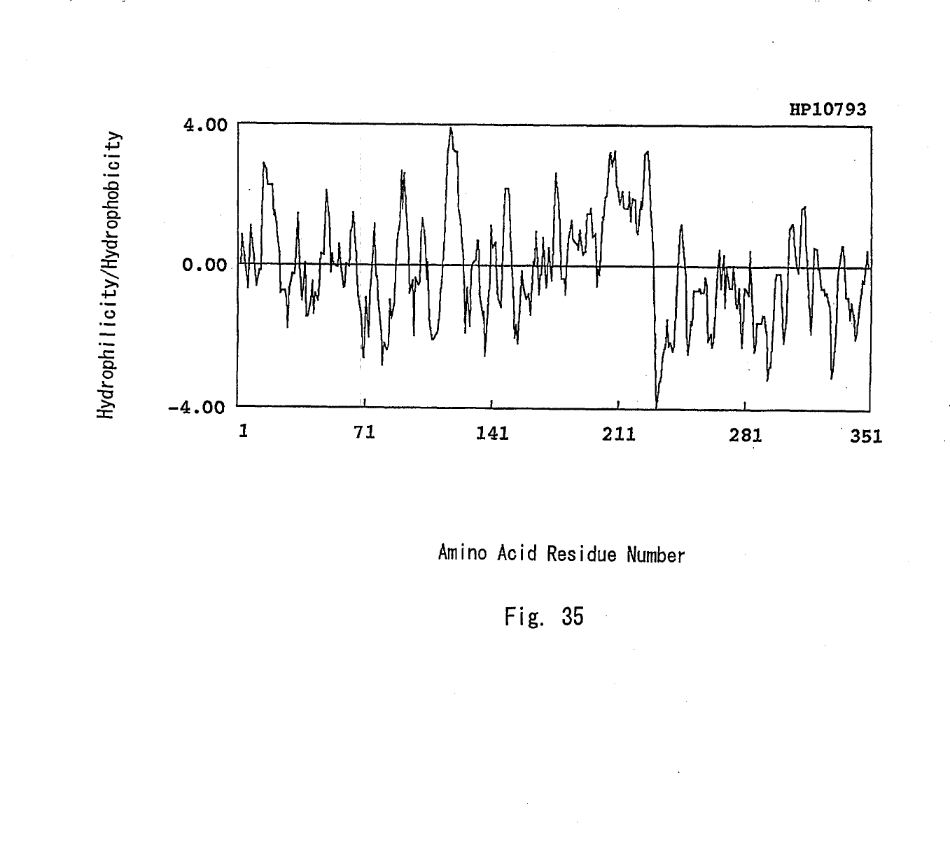

Determination of the whole base sequence of the cDNA insert of clone HP10793 obtained from cDNA library of human kidney revealed the structure consisting of a 70-bp 5′-untranslated region, a 1053-bp ORF, and a 206-bp 3′-untranslated region. The ORF encodes a protein consisting of 350 amino acid residues and there existed a putative secretory signal at the N-terminus and one putative transmembrane domain in the inner portion. FIG. 35 depicts the hydrophobicity/hydrophilicity profile, obtained by the Kyte-Doolittle method, of the present protein. In vitro translation resulted in formation of a translation product of 40 kDa that was somewhat larger than the molecular weight of 37,134 predicted from the ORF. Application of the (-3,-1) rule, a method for predicting the cleavage site of the secretory signal sequence, allows to expect that the mature protein starts from glycine-at position 25.

The search of the GenBank using the base sequences of the present cDNA has revealed the registration of sequences that shared a homology of 90% or more (for example, Accession No. AA326569) among ESTs. However, since they are partial sequences, it can not be judged whether or not they encode the same protein as the protein of the present invention.

<HP10794> (SEQ ID NOS: 96, 106, and 116)

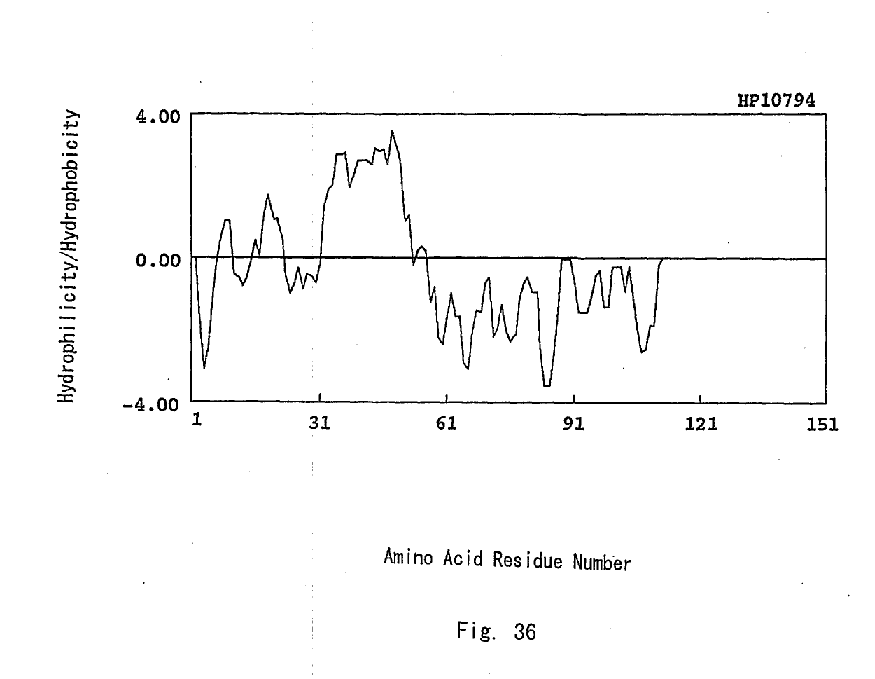

Determination of the whole base sequence of the cDNA insert of clone HP10794 obtained from cDNA library of human kidney revealed the structure consisting of a 146-bp 5′-untranslated region, a 342-bp ORF, and a 899-bp 3′-untranslated region. The ORF encodes a protein consisting of 113 amino acid residues and there existed one putative transmembrane domain. FIG. 36 depicts the hydrophobicity/hydrophilicity profile, obtained by the Kyte-Doolittle method, of the present protein. In vitro translation resulted in formation of a translation product of 14 kDa that was almost identical with the molecular weight of 12,017 predicted from the ORF.

The search of the GenBank using the base sequences of the present cDNA has revealed the registration of sequences that shared a homology of 90% or more (for example, Accession No. AI346561) among ESTs. However, since they are partial sequences, it can not be judged whether or not they encode the same protein as the protein of the present invention.

<HP10797> (SEQ ID NOS: 97, 107, and 117)

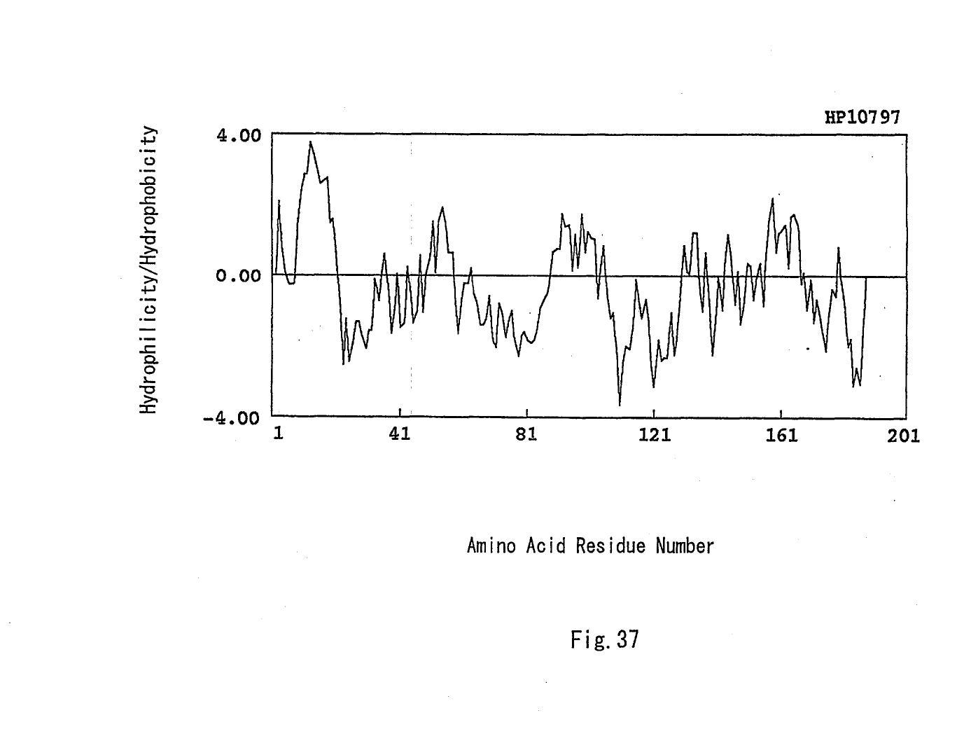

Determination of the whole base sequence of the cDNA insert of clone HP10797 obtained from cDNA library of human kidney revealed the structure consisting of a 129-bp 5′-untranslated region, a 570-bp ORF, and a 459-bp 3′-untranslated region. The ORF encodes a protein consisting of 189 amino acid residues and there existed a putative secretory signal at the N-terminus. FIG. 37 depicts the hydrophobicity/hydrophilicity profile, obtained by the Kyte-Doolittle method, of the present protein. In vitro translation resulted in formation of a translation product of 22 kDa that was almost identical with the molecular weight of 21,053 predicted from the ORF. Application of the (-3,-1) rule, a method for predicting the cleavage site of the secretory signal sequence, allows to expect that the mature protein starts from glutamine at position 23.

The search of the GenBank using the base sequences of the present cDNA has revealed the registration of sequences that shared a homology of 90% or more (for example, Accession No. AA356938) among ESTs. However, since they are partial sequences, it can not be judged whether or not they encode the same protein as the protein of the present invention. In addition, this gene was mapped on chromosome 4 (Accession No. AC004067).

<HP10798> (SEQ ID NOS: 98, 108, and 118)

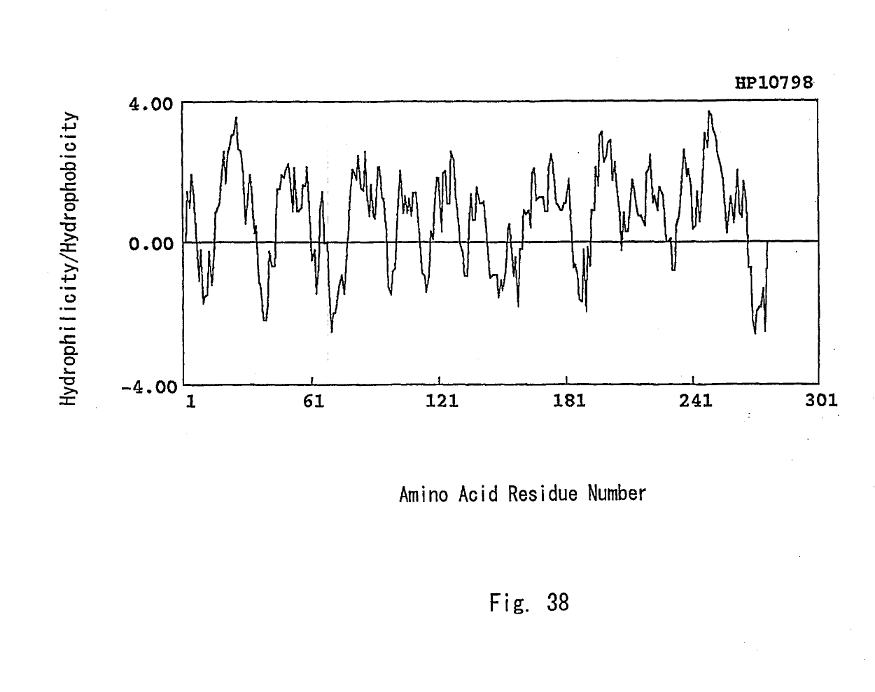

Determination of the whole base sequence of the cDNA insert of clone HP10798 obtained from cDNA library of human kidney revealed the structure consisting of a 25-bp 5′-untranslated region, a 834-bp ORF, and a 247-bp 3′-untranslated region. The ORF encodes a protein consisting of 277 amino acid residues and there existed seven putative transmembrane domains. FIG. 38 depicts the hydrophobicity/hydrophilicity profile, obtained by the Kyte-Doolittle method, of the present protein. In vitro translation resulted in formation of a translation product of 27 kDa that was smaller than the molecular weight of 30,685 predicted from the ORF.

The search of the GenBank using the base sequences of the present cDNA has revealed the registration of sequences that shared a homology of 90% or more (for example, Accession No. H92084) among ESTs. However, since they are partial sequences, it can not be judged whether or not they encode the same protein as the protein of the present invention.

<HP10800> (SEQ ID NOS: 99, 109, and 119)

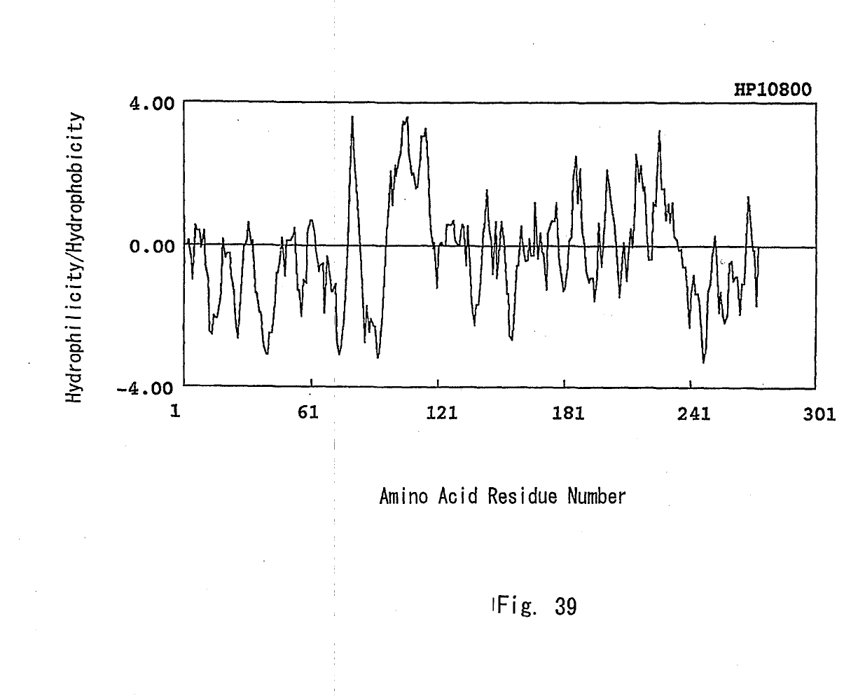

Determination of the whole base sequence of the cDNA insert of clone HP10800 obtained from cDNA library of human kidney revealed the structure consisting of a 158-bp 5′-untranslated region, a 825-bp ORF, and a 924-bp 3′-untranslated region. The ORF encodes a protein consisting of 274 amino acid residues and there existed one putative transmembrane domain. FIG. 39 depicts the hydrophobicity/hydrophilicity profile, obtained by the Kyte-Doolittle method, of the present protein. In vitro translation resulted in formation of a translation product of 33 kDa that was somewhat larger than the molecular weight of 31,108 predicted from the ORF. In this case, the addition of a microsome led to the formation of a product of 45 kDa. In addition, there exists in the amino acid sequence of this protein five sites at which N-glycosylation may occur (Asn-Ile-Thr at position 145, Asn-Ile-Thr at position 151, Asn-Ile-Thr at position 164, Asn-Ile-Thr at position 183, and Asn-Thr-Thr at position 256).

The search of the GenBank using the base sequences of the present cDNA has revealed the registration of sequences that shared a homology of 90% or more (for example, Accession No. AA729308) among ESTs. However, since they are partial sequences, it can not be judged whether or not they encode the same protein as the protein of the present invention.

<HP10801> (SEQ ID NOS: 100, 110, and 120)

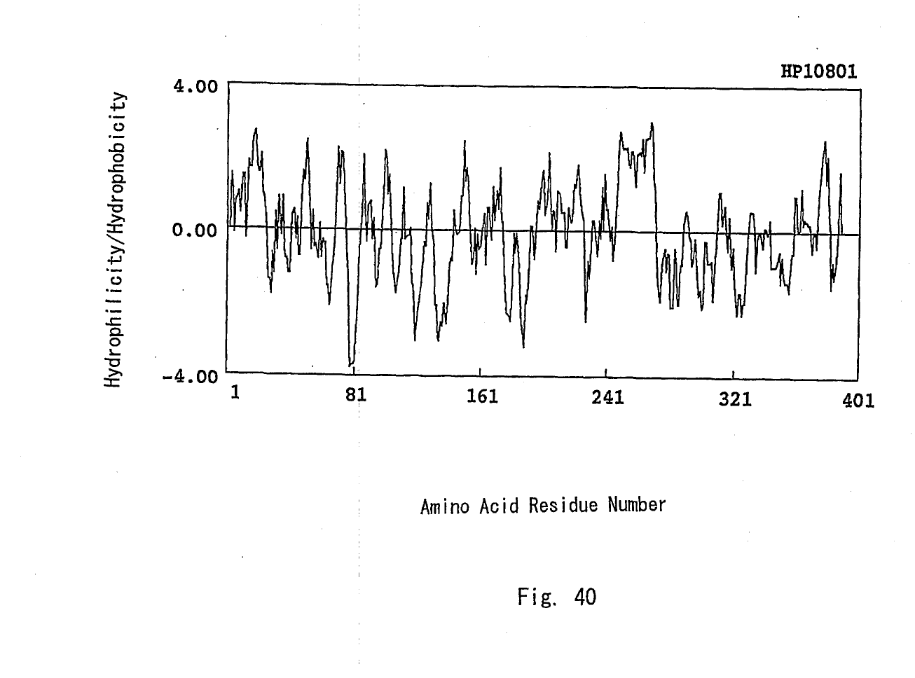

Determination of the whole base sequence of the cDNA insert of clone HP10801 obtained from cDNA library of human kidney revealed the structure consisting of a 133-bp 5′-untranslated region, a 1173-bp ORF, and a 510-bp 3′-untranslated region. The ORF encodes a protein consisting of 390 amino acid residues and there existed a putative secretory signal at the N-terminus and one putative transmembrane domain in the inner portion. FIG. 40 depicts the hydrophobicity/hydrophilicity profile, obtained by the Kyte-Doolittle method, of the present protein. In vitro translation with the addition of microsome resulted in formation of a product of 50 kDa that was larger than the molecular weight of 41,097 predicted from the ORF. In addition, there exists in the amino acid sequence of this protein five sites at which N-glycosylation may occur (Asn-Leu-Ser at position 108, Asn-Val-Thr at position 169, Asn-Leu-Ser at position 213, Asn-Val-Thr at position 236 and Asn-Gly-Thr at position 307). Application of the (-3,-1) rule, a method for predicting the cleavage site of the secretory signal sequence, allows to expect that the mature protein starts from glutamine at position 30.

The search of the protein database using the amino acid sequence of the present protein revealed that the protein was similar to human A33 antigen (Accession No. NP_{—}005805). Table 21 shows the comparison between amino acid sequences of the human protein of the present invention (HP) and human A33 antigen (HA). Therein, the marks of -, *, and . represent a gap, an amino acid residue identical with that of the protein of the present invention, and an amino acid residue similar to that of the protein of the present invention, respectively. The both proteins shared a homology of 28.7% in the intermediate region of 265 amino acod residues.

TABLE 21

| | |
|---|---|
| HP | MISLPGPLVTNLLRFLFLGLSALAPPSRAQLQLHLPANRLQAVEGGEVVLPAWY-TLHGE |
| | . .. *.. *.* .* .*.** * * .. |
| HA | MVGKMWPVLWTLCAVRVTVDAISVETPQDVLRASQGKSVTLPCTYHTSTSS |
| HP | VSSSQPWEVPFVMWFFKQKEKEDQVLSYINGVTTSKPGVSLVYSMPSRNLSLRLEGLQEK |
| | .. .*. .. . .**.* . * **. . .. *. .. * |
| HA | REGLIQWDKLLLTHTERVVIWPFSNKNYIHG-ELYKNRVSISNNAEQSDASITIDQLTMA |
| HP | DSGPYSCSVNVQDKQGKSRGHSIKTLELNVLVPPAPPSCRLQGVPHVGANVTLSCQSPRS |
| | *.*.*.***.. .. *.. . . * *****. *.* ..* . . *.*. *.*** . |
| HA | DNGTYECSVSL---MSDLEGNTKSRVRLLVLVPPSKPECGIEGETIIGNNIQLTCQSKEG |
| HP | KPAVQYQWDRQLPSFQTFFAPALDVIRGSLSLTNLSSSMAGVYVCKAHNEVGTAQCNVTL |
| | .*. ** *.* .. . * . ..**.*.*.. .* *.*.. ** **. **.*. |
| HA | SPTPQYSWKR-YNILNQEQPLAQPASGQPVSLKNISTDTSGYYICTSSNEEGTQFCNITV |
| HP | EV-STGPGAAVVAGAVVGTLVGLGLLAGLVLLYHCRGKALEEPANDIKEDAIAPRTLPWP |
| | .* *.. ..*. .* .**....* ... .. . **** . ... **** . * |
| HA | AVRSPSMNVALYVGIAVGVVAALIIIGIIIYCCCCRGK---DDNTEDKEDARPNREAYEE |
| HP | KSSDTISKNGTLSSVTSARALRPPHGPPRPFALTPTPSLSSQALPSPRLPTTDGAHPQPI |
| HA | PPEQLRELSREREEEDDYRQEEQRSTGRESPDHLDQ |

Furthermore, the search of the GenBank using the base sequences of the present cDNA has revealed the registration of sequences that shared a homology of 90% or more (for example, Accession No. R33685) among ESTS. However, since they are partial sequences, it can not be judged whether or not they encode the same protein as the protein of the present invention.

<HP03696> (SEQ ID NOS: 121, 131, and 141)

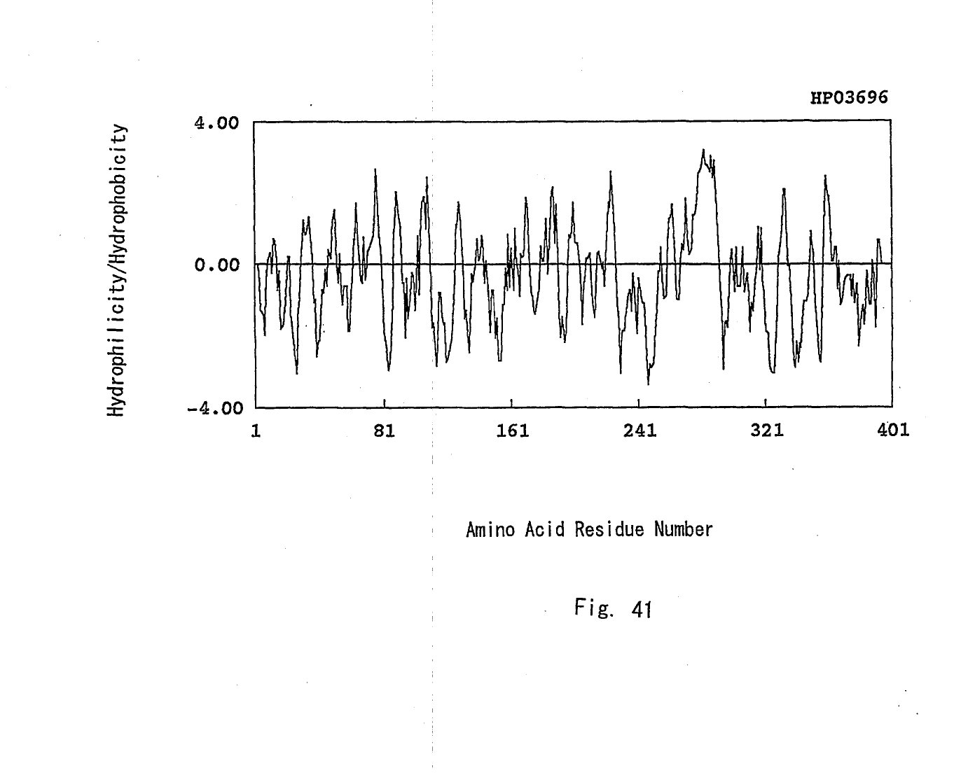

Determination of the whole base sequence of the cDNA insert of clone HP03696 obtained from cDNA library of human umbilical cord blood revealed the structure consisting of a 184-bp 5′-untranslated region, a 1188-bp ORF, and a 589-bp 3′-untranslated region. The ORF encodes a protein consisting of 395 amino acid residues and there existed one putative transmembrane domain. FIG. 41 depicts the hydrophobicity/hydrophilicity profile, obtained by the Kyte-Doolittle method, of the present protein.

The search of the protein database using the amino acid sequence of the present protein revealed that the protein was similar to rat cell surface glycoprotein GP42 (Accession No. P23505). Table 22 shows the comparison between amino acid sequences of the human protein of the present invention (HP) and rat cell surface glycoprotein GP42 (RC). Therein, the marks of -, *, and . represent a gap, an amino acid residue identical with that of the protein of the present invention, and an amino acid residue similar to that of the protein of the present invention, respectively. The both proteins shared a homology of 46.1% in the intermediate region of amino acid residues 62-280.

TABLE 22

| | |
|---|---|
| HP | MSGMEEYTTVSGEVLQRQKIPSFKENQTLSMGAATVQSRGQYSCSGQVMYIPQTFTQTSE |
| RC | MLLWMVLLLC |
| HP | TAMVQVQELFPPPVLSAIPSPEPREGSLVTLRCQTKLHPLRSALRLLFSFHKDGHTLQDR |
| | .*...****. **** . *.*... . *.* **..* ..* *..**.**.*..*.* |
| RC | VSMTEAQELFQDPVLSRLNSSETSD---LLLKCTTKVDPNKPASELFYSFYKDNHIIQNR |
| HP | GPHPELCIPGAKEGDSGLYWCEVAPEGGQVQKQSPQLEVRVQAPVSRPVLTLHHGPADPA |
| | ...* . *..*.*..**** * *....* .**.* *.. . ..**.*****.*.... * |
| RC | SHNPLFFISEANEENSGLYQCVVDAKDGTIQKKSDYLDIDLCTSVSQPVLTLQHEATNLA |
| HP | VGDMVQLLCEAQRGSPPILYSFYLDELIVGNHSAPCGGTTSLLFPVKSEQDAGNYSCEAE |
| | **.*..***.* ** *******.*..*.*.. **.* ..***..**.* .. ****.** |
| RC | EGDKVKFLCETQLGSLPILYSFYMDGEILGEPLAPSGRAASLLISVKAEWSGKNYSCQAE |
| HP | NSVSRERSEPKKLSLKGSQVLFTPASNWLVPWLPAS-LLGLMVIAAALLVYVRSWRKAGP |
| | *.***. *****..* * . ... .* ***.* *.* |
| RC | NKVSRDISEPKKFPLVVSGTASMKSTT-VVIWLPVSCLVGWPWLLRF |

Furthermore, the search of the GenBank using the base sequences of the present cDNA has revealed the registration of sequences that shared a homology of 90% or more (for example, Accession No. AA446524) among ESTs. However, since they are partial sequences, it can not be judged whether or not they encode the same protein as the protein of the present invention.

<HP03882> (SEQ ID NOS: 122, 132, and 142)

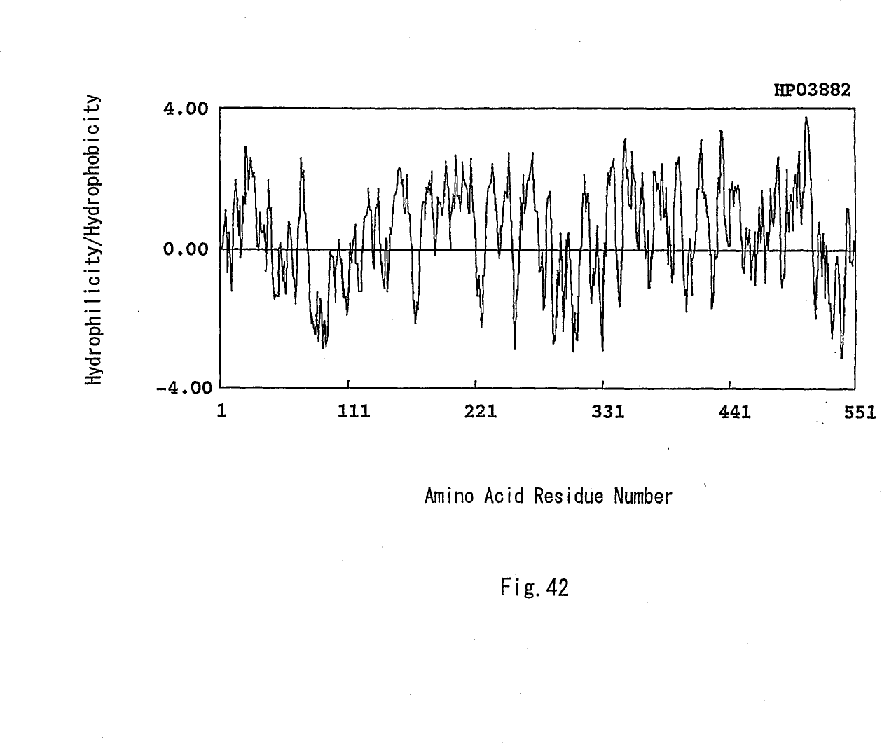

Determination of the whole base sequence of the cDNA insert of clone HP03882 obtained from cDNA library of human kidney revealed the structure consisting of a 57-bp 5 untranslated region, a 1653-bp ORF, and a 484-bp 3′-untranslated region. The ORF encodes a protein consisting of 550 amino acid residues and there existed ten putative transmembrane domains. FIG. 42 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to mouse solute carrier family 22 (cation transporter)-like protein (Accession No. NP_{—}033229). Table 23 shows the comparison between amino acid sequences of the human protein of the present invention (HP) and mouse solute carrier family 22 (cation transporter)-like protein (MS). Therein, the marks of -, *, and . represent a gap, an amino acid residue identical with that of the protein of the present invention, and an amino acid residue similar to that of the protein of the present invention, respectively. The both proteins shared a homology of 48.9% in the entire region.

TABLE 23

| | |
|---|---|
| HP | MAFSKLLEQAGGVGLFQTLQVLTFILPCLMIPSQMLLENFSAAIPGHRCWTHMLDN---G |
| | ***..**...**.* ** .*..... * * ...* .*******.* ****...*** |
| MS | MAFPELLDRVGGLGRFQLFQTVALVTPILWVTTQNMLENFSAAVPHHRCWVPLLDNSTSQ |
| HP | SAVSTNMTPKALLTISIPPGPNQGPHQCRRFRQPQWQLLDPNATATSWSEADTEPCVDGW |
| | ........*..**..******.* **** ********* ..*****.**.*.**** *** |
| MS | ASIPGDLGPDVLLAVSIPPGPDQQPHQCLRFRQPQWQLTESNATATNWSDAATEPCEDGW |
| HP | VYDRSVFTSTIVAKWDLVCSSQGLKPLSQSIFMSGILVGSFIWGLLSYRFGRKPMLSWCC |
| | ***.*.* ****..*****.**.*.*..****..*****. . * * ****...*.*.. |
| MS | VYDHSTFRSTIVTTWDLVCNSQALRPMAQSIFLAGILVGAAVCGHASDRFGRRRVLTWSY |
| HP | LQLAVAGTSTIFAPTFVIYCGLRFVAAFGMAGIFLSSLTLMVEWTTTSRRAVTMTVVGCA |
| | * ..*.**.. * *** .** .**. * ..**..... .*..***.. ....**. . . |
| MS | LLVSVSGTAAAFMPTFPLYCLFRFLLASAVAGVMMNTASLLMEWTSAQGSPLVMTLNALG |
| HP | FSAGQAALGGLAFALRDWRTLQLAASVPFFAISLISWWLPESARWLIIKGKPDQALQELR |
| | ** **. *..*...*.** ****.*.*** . . ************. ** **.****. |
| MS | FSFGQVLTGSVAYGVRSWRMLQLAVSAPFFLFFVYSWWLPESARWLITVGKLDQGLQELQ |
| HP | KVARINGHK-EAKNLTIEVLMSSVKEEVASAKEPRSVLDLFCVPVLRWRSCAMLVVNFSL |
| | .** .* .* *...**.***.*...** ...*. *. .*. .* ** *. .. *.. |
| MS | RVAAVNRRKAEGDTLTMEVLRSAMEEEPSRDKAGASLGTLLHTPGLRHRTIISMLCWFAF |
| HP | LISYYGLVFDLQSLGRDIFLLQALFGAVDFLGRATTALLLSFLGRRTIQAGSQAMAGLAI |
| | ...***..***.**..*******.* *** .... **.* **** *.. ...** * |
| MS | GFTFYGLALDLQALGSNIFLLQALIGIVDFPVKTGSLLLISRLGRRLCQVSFLVLPGLCI |
| HP | LANMLVPQDLQTLRVVFAVLGKGCFGISLTCLTIYKAELFPTPVRMTADGILHTVGRLGA |
| | *.*.***... .** ..**** **.* ..**.**...***** .**** *. ....* ** |
| MS | LSNILVPHGMGVLRSALAVLGLGCLGGAFTCITIFSSELFPTVIRMTAVGLCQVAARGGA |
| HP | MMGPLILMSRQALPLLPPLLYGVISIASSLVVLFFLPETQGLPLPDTIQDLESQKSTAAQ |
| | *.***. . . .* *.***... *.*..* .****..*********...*. . . |
| MS | MLGPLVRLLGVYGSWMPLLVYGVVPVLSGLAAL-LLPETKNLPLPDTIQDIQKQSYKKVT |
| HP | GNRQEAVTVESTSL |
| | . ... .. **.* |
| MS | HDTPDGSILMSTRL |

The search of the GenBank using the base sequences of the present cDNA has revealed the registration of sequences that shared a homology of 90% or more (for example, Accession No. AI242210) among ESTs. However, since they are partial sequences, it can not be judged whether or not they encode the same protein as the protein of the present invention.

<HP03903> (SEQ ID NOS: 123, 133, and 143)

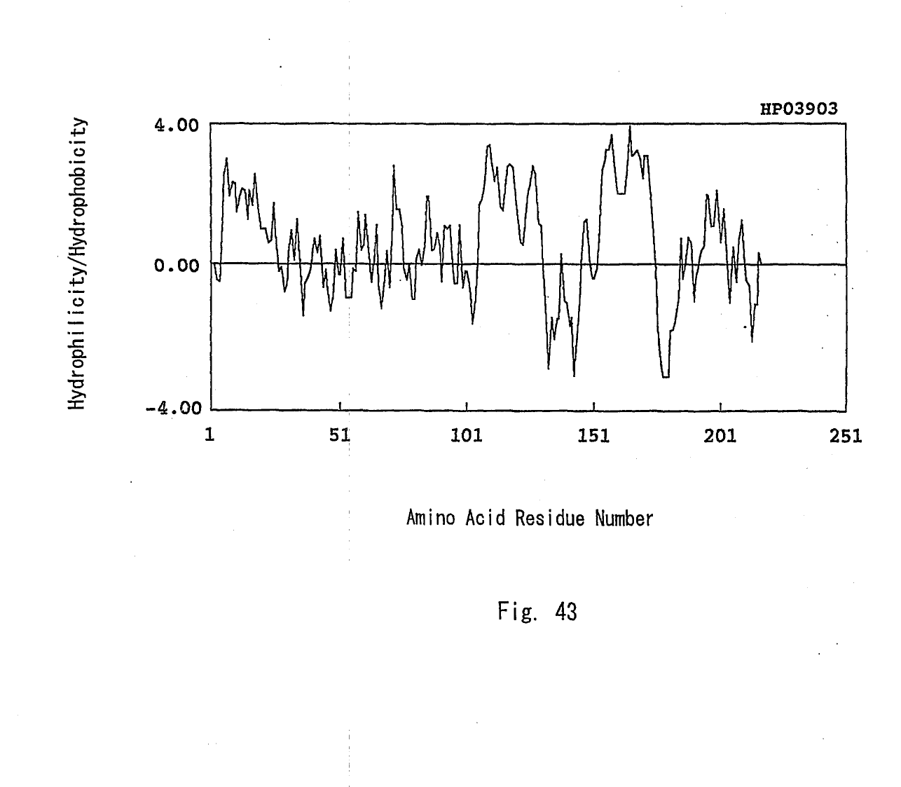

Determination of the whole base sequence of the cDNA insert of clone HP03903 obtained from cDNA library of human kidney revealed the structure consisting of a 108-bp 5′-untranslated region, a 657-bp ORF, and a 1988-bp 3′-untranslated region. The ORF encodes a protein consisting of 218 amino acid residues and there existed three putative transmembrane domains. FIG. 43 depicts the hydrophobicity/hydrophilicity profile, obtained by the Kyte-Doolittle method, of the present protein. In vitro translation resulted in formation of a translation product of 26 kDa that was somewhat larger than the molecular weight of 23,487 predicted from the ORF.

The search of the protein database using the amino acid sequence of the present protein revealed that the protein was similar to mouse prominin (Accession No. NP_{—}032961). Table 24 shows the comparison between amino acid sequences of the human protein of the present invention (HP) and mouse prominin (MP). Therein, the marks of -, *, and . represent a gap, an amino acid residue identical with that of the protein of the present invention, and an amino acid residue similar to that of the protein of the present invention, respectively. The both proteins shared a homology of 27.6% in the region other than the N-terminal and C-terminal regions.

TABLE 24

| | |
|---|---|
| HP | MKHTLALLAPLLGLGLGLALSQLAAGATDCKFLGPAEHLTFTPAARARWLAPRVRAPGLL |
| | .* ... * *.*. ..... * .... .... .. ... .. . ..*.. |
| MP | MALVFSALLLLGLCGKISSEGQPAFHNTPGAMNYELPT-TKYETQDTFNAGIV |
| HP | DSLYGTVRRFLSVVQLNPFPSELVKALL--NELA-SVKVNEVVRYEAGYVVCAVIAGLYL |
| | ..** *. **.*** * ** .*. * *. .... **. .*.. ** * ..**... *.. |
| MP | GPLYKMVHIFLNYVQPNDFPLDLIKKLIQNKNFDISVDSKEIALYEIGVLICAILGLLFI |
| HP | LLVPTAGLCFCCCRCHRRCGGRVKTEHK-ALACERAALMVFLLLTTLLLLIGVVCAFVTN |
| | .*.* .* ** *** ..***.. . * . .* * * . **.. **. L*....**.* |
| MP | ILMPLVGCFFCMCRCCNKCGGEMHQRQKQNAPCRRKCLGLSLLVICLLMSLGIIYGFVAN |
| HP | QRTHEQMGPSIEAMPETLLSLWGLVSDVPQVSTVTPHPHVPL |
| | *.*.... . . ... .. .*....*. |
| MP | QQTRTRIKGTQKLAKSNFRDFQTLLTETPKQIDYVVEQYTNTKNKAFSDLDGIGSVLGGR |

The search of the GenBank using the base sequences of the present cDNA has revealed the registration of sequences that shared a homology of 90% or more (for example, Accession No. AI792608) among ESTs. However, since they are partial sequences, it can not be judged whether or not they encode the same protein as the protein of the present invention.

<HP03974> (SEQ ID NOS: 124, 134, and 144)

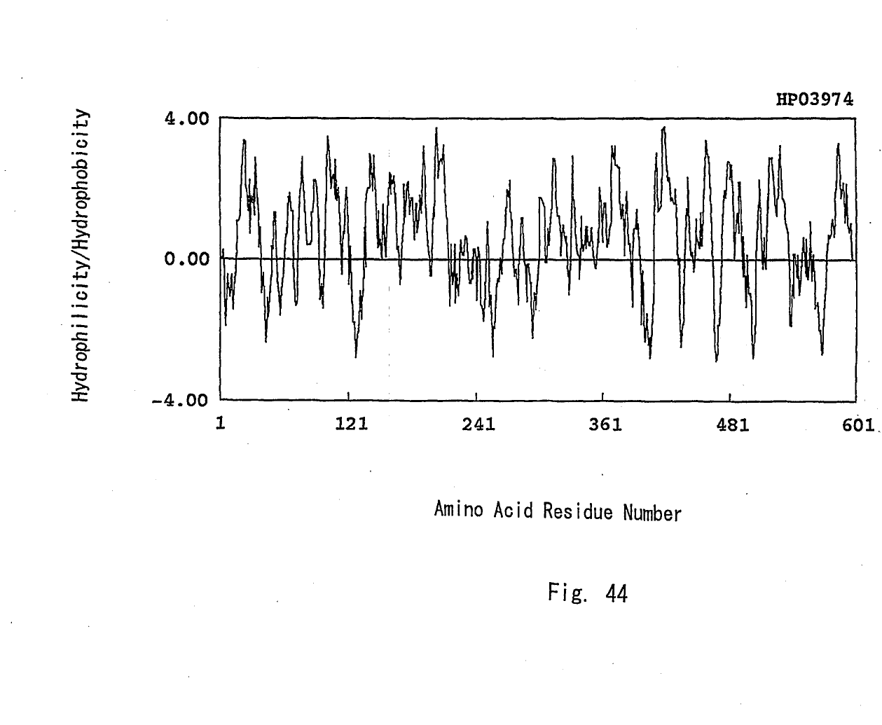

Determination of the whole base sequence of the cDNA insert of clone HP03974 obtained from cDNA library of human kidney revealed the structure consisting of a 41-bp 5′-untranslated region, a 1791-bp ORF, and a 253-bp 3′-untranslated region. The ORF encodes a protein consisting of 596 amino acid residues and there existed twelve putative transmembrane domains. FIG. 44 depicts the hydrophobicity/hydrophilicity profile, obtained by the Kyte-Doolittle method, of the present protein. In vitro translation resulted in formation of a translation product of high molecular weight.

The search of the protein database using the amino acid sequence of the present protein revealed that the protein was similar to rabbit (Oryctolagus cuniculus) sodium/glucose cotransporter protein (Accession No. AAA66065). Table 25 shows the comparison between amino acid sequences of the human protein of the present invention (HP) and rabbit sodium/glucose cotransporter protein (OC). Therein, the marks of -, *, and . represent a gap, an amino acid residue identical with that of the protein of the present invention, and an amino acid residue similar to that of the protein of the present invention, respectively. The both proteins shared a homology of 89.1% in the entire region.

TABLE 25

| | |
|---|---|
| HP | M-AANSTSDLHTPGTQLSVADIIVITVYFALNVAVGIWSSCRASRNTVNGYFLAGRDMTW |
| | * *.***** *.**.****.**.************************.************ |
| OC | MVADNSTSDPHAPGPQLSVTDIVVITVYFALNVAVGIWSSCRASRNTVSGYFLAGRDMTW |
| HP | WPIGASLFASSEGSGLFIGLAGSGAAGGLAVAGFEWNATYVLLALAWVFVPIYISSEIVT |
| | ********.*************************.************** .********* |
| OC | WPIGASLFGSSEGSGLFIGLAGSGAAGGLAVAGFDWNATYVLLALAWVFGAIYISSEIVT |
| HP | LPEYIQKRYGGQRIRMYLSVLSLLLSVFTKISLDLYAGALFVHICLGWNFYLSTILTLGI |
| | *.******.*************************************************.* |
| OC | LAEYIQKRFGGQRIRMYLSVLSLLLSVFTKISLDLYAGALFVHICLGWNFYLSTILTLTI |
| HP | TALYTIAGGLAAVIYTDALQTLIMVVGAVILTIKAFDQIGGYGQLEAAYAQAIPSRTIAN |
| | ******.***.********************.****.**.****.*****.******.** |
| OC | TALYTITGGLVAVIYTDALQTLIMVVGAVILAIKAFHQIDGYGQMEAAYARAIPSRTVAN |
| HP | TTCHLPRTDAMHMFRDPHTGDLPWTGMTFGLTIMATWYWCTDQVIVQRSLSARDLNHAKA |
| | *******.*********.***********************************.****** |
| OC | TTCHLPRADAMHMFRDPYTGDLPWTGMTFGLTIMATWYWCTDQVIVQRSLSARNLNHAKA |
| HP | GSILASYLKMLPMGLIIMPGMISRALFPDDVGCVVPSECLRACGAEVGCSNIAYPKLVME |
| | ***************.*************.****************.************* |
| OC | GSILASYLKMLPMGLMIMPGMISRALFPDEVGCVVPSECLRACGAEIGCSNIAYPKLVME |
| HP | LMPIGLRGLMIAVMLAALMSSLTSIFNSSSTLFTMDIWRRLRPRSGERELLLVGRLVIVA |
| | ***.**********..******.******************** ..*************. |
| OC | LMPVGLRGLMIAVMMPALMSSLSSIFNSSSTLFTMDIWRRLRPCASERELLLVGRLVIVV |
| HP | LIGVSVAWIPVLQDSNSGQLFIYMQSVTSSLAPPVTAVFVLGVFWRRANEQGAFWGLIAG |
| | *************.**.**********************.**.**.***********.** |
| OC | LIGVSVAWIPVLQGSNGGQLFIYMQSVTSSLAPPVTAVFTLGIFWQRANEQGAFWGLLAG |
| HP | LVVGATRLVLEFLNPAPPCGEPDTRPAVLGSIHYLHFAVALFALSGAVVVAGSLLTPPPQ |
| | *.***********.******..*******. .**********.*.***.*.********. |
| OC | LAVGATRLVLEFLHPAPPCGAADTRPAVLSQLHYLHFAVALFVLTGAVAVGGSLLTPPPR |
| HP | SVQIENLTWWTLAQDVPLGTKAGDGQTPQKHAFWARVCGFNAILLMCVNIFFYAYFA |
| | **********..*..**.*********...************************* |
| OC | RHQIENLTWWTLTRDLSLGAKAGDGQTPQRYTFWARVCGFNAILLMCVNIFFYAYFA |

The search of the GenBank using the base sequences of the present cDNA has revealed the registration of sequences that shared a homology of 90% or more (for example, Accession No. AI793336) among ESTs. However, since they are partial sequences, it can not be judged whether or not they encode the same protein as the protein of the present invention.

<HP03978> (SEQ ID NOS: 125, 135, and 145)

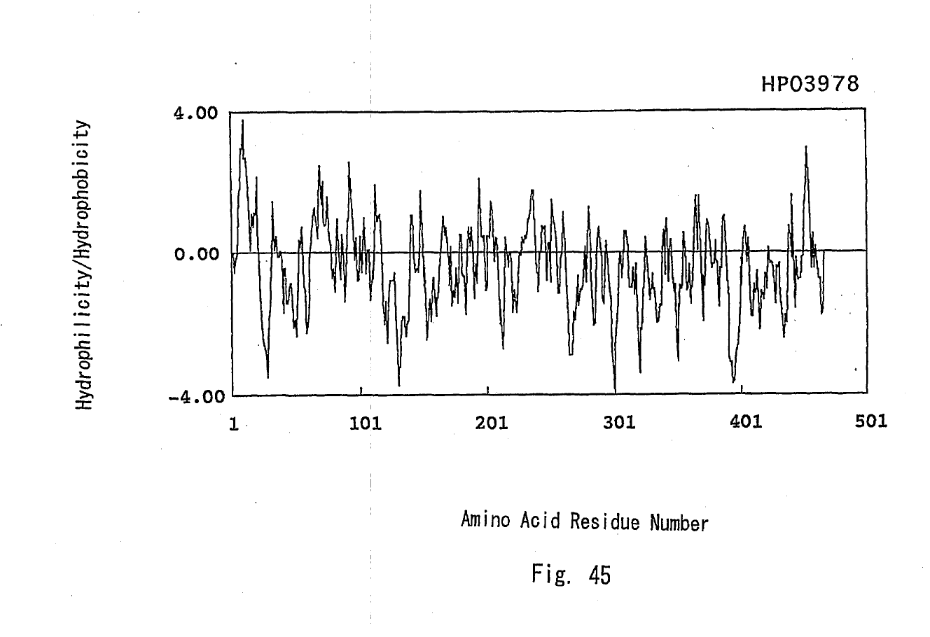

Determination of the whole base sequence of the cDNA insert of clone HP03978 obtained from cDNA library of human kidney revealed the structure consisting of a 99-bp 5′-untranslated region, a 1404-bp ORF, and a 705-bp 3′-untranslated region. The ORF encodes a protein consisting of 467 amino acid residues and there existed a putative secretory signal at the N-terminus. FIG. 45 depicts the hydrophobicity/hydrophilicity profile, obtained by the Kyte-Doolittle method, of the present protein. In vitro translation resulted in formation of a translation product of 55 kDa that was somewhat larger than the molecular weight of 52,352 predicted from the ORF. In this case, the addition of a microsome led to the formation of a product of 57 kDa. In addition, there exists in the amino acid sequence of this protein two sites at which N-glycosylation may occur (Asn-Arg-Thr at position 78 and Asn-His-Ser at position 161). Application of the (-3,-1) rule, a method for predicting the cleavage site of the secretory signal sequence, allows to expect that the mature protein starts from alanine at position 22.

The search of the protein database using the amino acid sequence of the present protein revealed that the protein was similar to human tubulo-interstitial nephritis antigen (Accession No. BAA84949). Table 26 shows the comparison between amino acid sequences of the human protein of the present invention (HP) and human tubulo-interstitial nephritis an gen (TA). Therein, the marks of -, *, and . represent a gap, an amino acid residue identical with that of the protein of the present invention, and an amino acid residue similar to that of the protein of the present invention, respectively. The both proteins shared a homology of 50.0% in the region other than the N-terminal region.

TABLE 26

| | |
|---|---|
| HP | MWRCPLGLLLLLPLAGHLALGAQQGRGRRELAPGLHLRGIRDAGGRYCQEQ |
| | *.**.. |
| TA | MWTGYKILIFSYLTTEIWMEKQYLSQREVDLEAYFTRNHTVLQGTRFKRAIFQGQYCRNF |
| HP | DLCCRGRADDCALP-YLG-AICYCDLFCNRTVSDCCPDFWDFC---LGVPPPFPP--IQG |
| | . ** .*.*.*. * . *.**** **.*. ******. .** . **. .* .* |
| TA | G-CCEDRDDGCVTEFYAANALCYCDKFCDRENSDCCPDYKSFCREEKEWPPHTQPWYPEG |
| HP | CMHGGRIYPVLGTYWDNCNRCTCQENRQWQCDQEPCLVDPDMIKAINQGNYGWQAGNHSA |
| | *...*. * .. .***.*** ...**.*.*. *** *..*...*.*.*** * *.*. |
| TA | CFKDGQHYEEGSVIKENCNSCTC-SGQQWKCSQHVCLVRPELIEQVNKGDYGWTAQNYSQ |
| HP | FWGMTLDEGIRYRLGTIRPSSSVMNMHEIYTVLNPGEVLPTAFEASEKWPNLIHEPLDQG |
| | ******..*...****..** ...*.*. . * ... **. * ** ***. . *.**** |
| TA | FWGMTLEDGFKFRLGTLPPSLMLLSMNEMTASLPATTDLPEFFVASYKWPGWTHGPLDQK |
| HP | NCAGSWAFSTAAVASDRVSIHSLGHMTPVLSPQNLLSCDTHQQQGCRGGRLDGAWWFLRR |
| | ***.*******.**.**..*.* *. *. ******.** .....**..*..* ***.**. |
| TA | NCAASWAFSTASVAADRIAIQSKGRYTANLSPQNLISCCAKNRHGCNSGSIDRAWWYLRK |
| HP | RGVVSDHCYPFSGRERDEAGPAPPCMMHSRAMGRGKRQATAHCPNSYVNNNDIYQVTPVY |
| | **.**. ***. .*..... * * **. *****.** .***. ..* *** .* * |
| TA | RGLVSHACYPLF---KDQNATNNGCAMASRSDGRGKRHATKPCPNNVEKSNRIYQCSPPY |
| HP | RLGSNDKELMKELMENGPVQALMEVHEDFFLYKGGIYSHTPVSLGRPERYRRHGTHSVKI |
| | *..**..*****.*.******.*.****** **.***.*.. . .*.**. **.**. |
| TA | RVSSNETEIMKEIMQNGPVQAIMQVHEDFFHYKTGIYRIWTSTNKESEKYRKLQTHAVKL |
| HP | TGWGEETLPDGRTLKYWTAANSWGPAWGERGHFRIVRGVNECDIESFVLGYWGRVGMEDM |
| | ****. ..*.. *.*.****** .***.*.***.*****.***......**... .* |
| TA | TGWGTLRGAQGQKEKFWIAANSWGKSWGENGYFRILRGVNESDIEKLIIAAWGQLTSSDE |
| HP | GHH |
| TA | P |

The search of the GenBank using the base sequences of the present cDNA has revealed the registration of sequences that shared a homology of 90% or more (for example, Accession No. R48402) among ESTs. However, since they are partial sequences, it can not be judged whether or not they encode the same protein as the protein of the present invention.

<HP10735> (SEQ ID NOS: 126, 136, and 146)

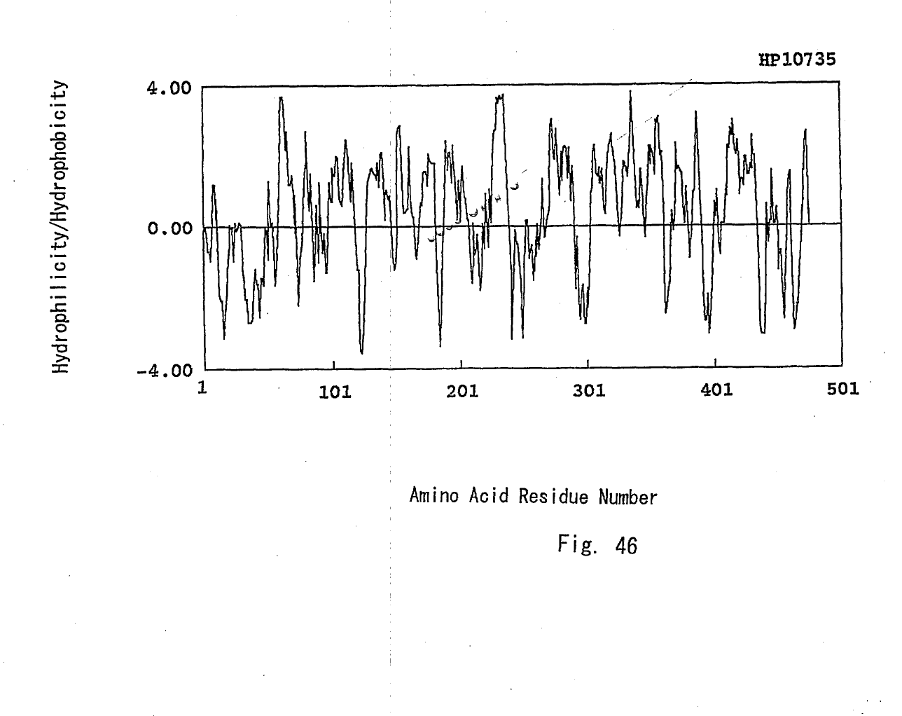

Determination of the whole base sequence of the cDNA insert of clone HP10735 obtained from cDNA library of human umbilical cord blood revealed the structure consisting of a 370-bp 5′-untranslated region, a 1431-bp ORF, and a 243-bp 3′-untranslated region. The ORF encodes a protein consisting of 476 amino acid residues and there existed ten putative transmembrane domains. FIG. 46 depicts the hydrophobicity/hydrophilicity profile, obtained by the Kyte-Doolittle method, of the present protein.

The search of the protein database using the amino acid sequence of the present protein revealed that the protein was similar to Caenorhabditis elegans tetracycline resistance protein-like protein (Accession No. CAA94337). Table 27 shows the comparison between amino acid sequences of the human protein of the present invention (HP) and C. elegans tetracycline resistance protein-like protein (CP). Therein, the marks of -, *, and . represent a gap, an amino acid residue identical with that of the protein of the present invention, and an amino acid residue similar to that of the protein of the present invention, respectively. The both proteins shared a homology of 51.5% in the intermediate region of 196 amino acid residues.

TABLE 27

| | |
|---|---|
| HP | MAGSDTAPFLSQADDPDDGPVPGTPGLPGSTGNPKSEEPEVPDQEGLQRITGLSPGRSAL |
| | ... .. . |
| CP | MVNSQQDYI |
| HP | IVAVLCYINLLNYMDRFTVAGVLPDIEQFFNIGDSSSGLIQTVFISSYMVLAPVFGYLGD |
| | *..* .*****.**.******.... ..**.** .*******. *.**..** ***** |
| CP | SVTALFVVNLLNYVDRYTVAGVLTQVQTYYNISDSLGGLIQTVFLISFMVFSPVCGYLGD |
| HP | RYNRKYLMCGGIAFWSLVTLGSSFIPGEHFWLLLLTRGLVGVGEASYSTIAPTLIADLFV |
| | *.***..* *...* ..*****.*..****.*. *..**.******..**.**.*.* |
| CP | RFNRKWIMIIGVGIWLGAVLGSSFVPANHFWLFLVLRSFVGIGEASYSNVAPSLISDMFN |
| HP | ADQRSRMLSIFYFAIPVGSGLGYIAGSKVKDMAGDWHWALRVTPGLGVVAVLLLFLVVRE |
| | ...** .. *************.*.**.* ...*.*.*..**.. *..... * * . * |
| CP | GQKRSTVFMIFYFAIPVGSGLGFIVGSNVATLTGHWQWGIRVSAIAGLIVMIALVLFTYE |
| HP | PPRGAVERHSDLPPLNPTSWWADLRALARNLIFGLITCLTGVLGVGLGVEISRRLRHSNP |
| | * ***... |
| CP | PERGAADKAMGESKDVVVTTNTTYLEDLVILLKTPTLVACTWGYTALVFVSGTLSWWEPT |
| HP | RADPLVCATGLLGSAPFLFLSLACARGSIVATYIFIFIGETLLSMNWAIVADILLYVVIP |
| CP | VIQHLTAWHQGLNDTKDLASTDKDRVALYFGAITTAGGLIGVIFGSMLSKWLVAGWGPFR |
| HP | TRRSTAEAFQIVLSHLLGDAGSPYLIGLISDRLRRNWPPSFLSEFRALQFSLMLCAFVGA |
| CP | RLQTDRAQPLVAGGGALLAAPFLLIGMIFGDKSLVLLYIMIFFGITFMCFNWGLNIDMLT |
| HP | LGGAAFLGTAIFIEADRRRAQLHVQGLLHEAGSTDDRIVVPQRGRSTRVPVASVLI |
| CP | TVIHPNRRSTAFSYFVLVSHLFGDASGPYLIGLISDAIRHGSTYPKDQYHSLVSATYCCV |

The search of the GenBank using the base sequences of the present cDNA has revealed the registration of sequences that shared a homology of 90% or more (for example, Accession No. AA460778) among ESTs. However, since they are partial sequences, it can not be judged whether or not they encode the same protein as the protein of the present invention. Furthermore, the search has revealed the registration of sequences that shared a homology of 90% or more (Accession No. E12646) in patent data. However, since they are partial sequences, it can not be judged whether or not they encode the same protein as the protein of the present invention.

<HP10750> (SEQ ID NOS: 127, 137, and 147)

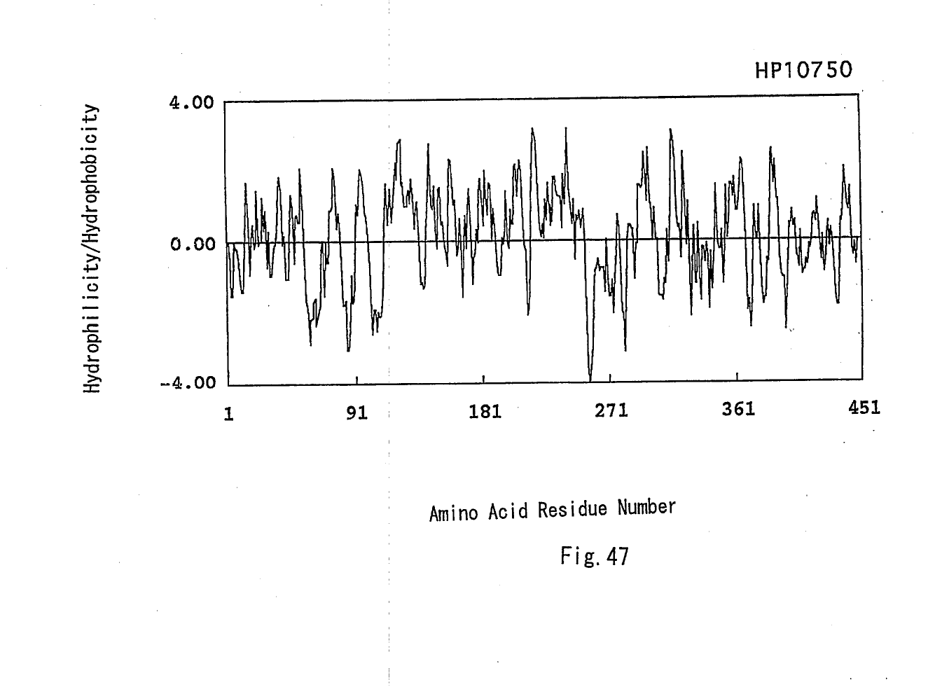

Determination of the whole base sequence of the cDNA insert of clone HP10750 obtained from cDNA library of human umbilical cord blood revealed the structure consisting of a 262-bp 5′-untranslated region, a 1350-bp ORF, and a 564-bp 3′-untranslated region. The ORF encodes a protein consisting of 449 amino acid residues and there existed four putative transmembrane domains. FIG. 47 depicts the hydrophobicity/hydrophilicity profile, obtained by the Kyte-Doolittle method, of the present protein.

The search of the GenBank using the base sequences of the present cDNA has revealed the registration of sequences that shared a homology of 90% or more (for example, Accession No. AW304031) among ESTs. However, since they are partial sequences, it can not be judged whether or not they encode the same protein as the protein of the present invention.

<HP10777> (SEQ ID NOS: 128, 138, and 148)

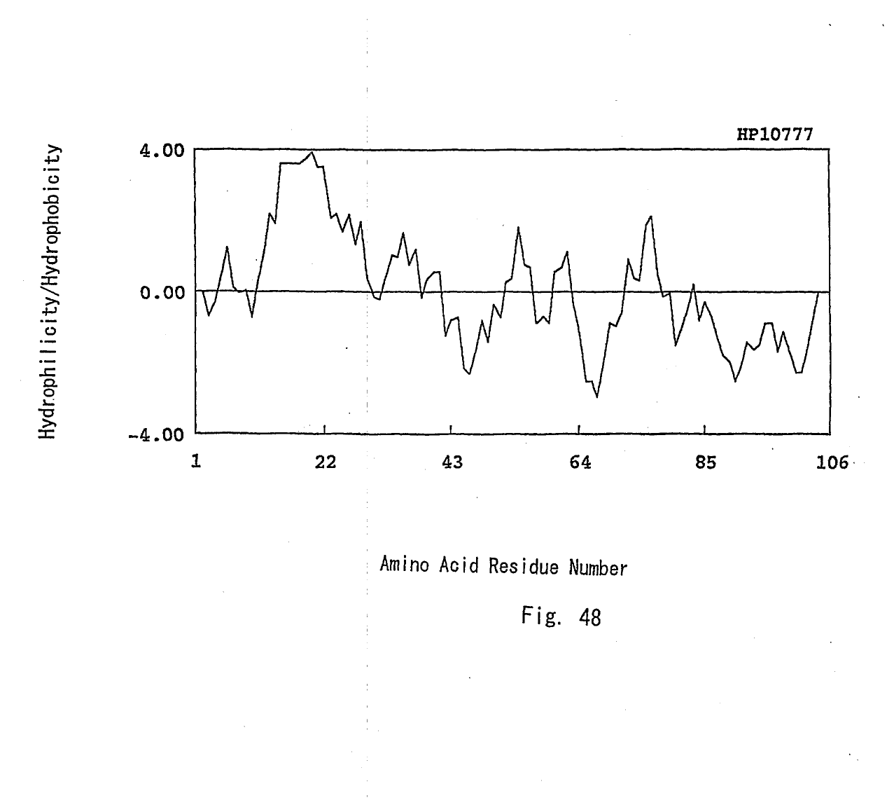

Determination of the whole base sequence of the cDNA insert of clone HP10777 obtained from cDNA library of human kidney revealed the structure consisting of a 15-bp 5′-untranslated region, a 318-bp ORF, and a 1030-bp 3′-untranslated region. The ORF encodes a protein consisting of 105 amino acid residues and there existed a putative secretory signal at the N-terminus. FIG. 48 depicts the hydrophobicity/hydrophilicity profile, obtained by the Kyte-Doolittle method, of the present protein. In vitro translation resulted in formation of a translation product of 14 kDa that was somewhat larger than the molecular weight of 11,603 predicted from the ORF. Application of the (-3,-1) rule, a method for predicting the cleavage site of the secretory signal sequence, allows to expect that the mature protein starts from glycine at position 30.

<HP10780> (SEQ ID NOS: 129, 139, and 149)

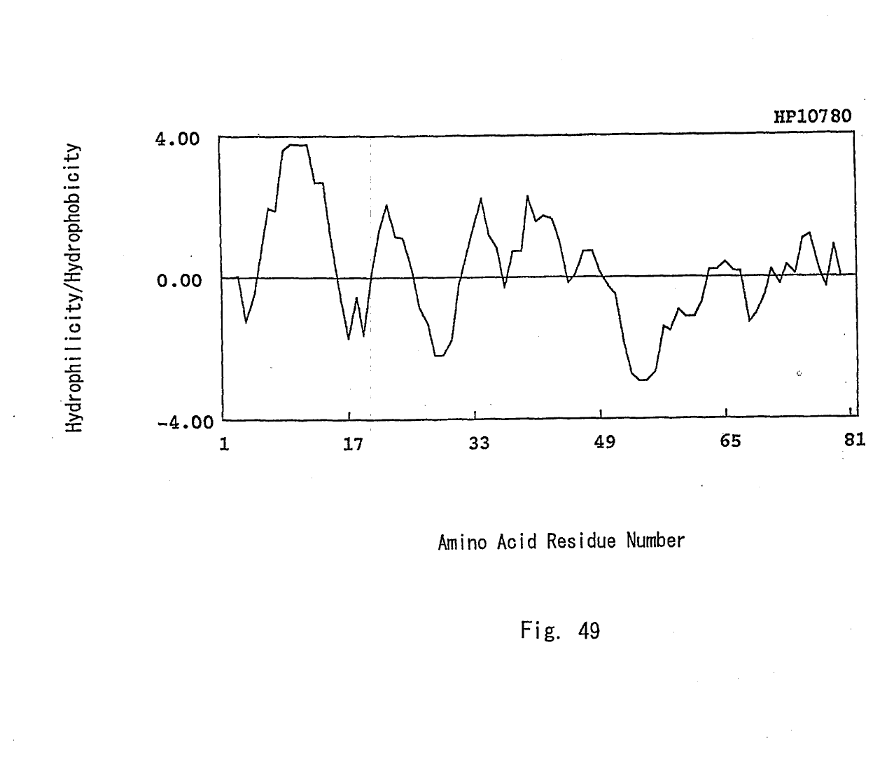

Determination of the whole base sequence of the cDNA insert of clone HP10780 obtained from cDNA library of human kidney revealed the structure consisting of a 226-bp 5′-untranslated region, a 246-bp ORF, and a 571-bp 3′-untranslated region. The ORF encodes a protein consisting of 81 amino acid residues and there existed a putative secretory signal at the N-terminus. FIG. 49 depicts the hydrophobicity/hydrophilicity profile, obtained by the Kyte-Doolittle method, of the present protein. In vitro translation resulted in formation of a translation product of 10 kDa that was somewhat larger than the molecular weight of 8,533 predicted from the ORF. In this case, the addition of a microsome led to the formation of a product of 6 kDa. Application of the (-3,-1) rule, a method for predicting the cleavage site of the secretory signal sequence, allows to expect that the mature protein starts from glycine at position 25.

The search of the GenBank using the base sequences of the present cDNA has revealed the registration of sequences that shared a homology of 90% or more (for example, Accession No. AA658245) among ESTs. However, since they are partial sequences, it can not be judged whether or not they encode the same protein as the protein of the present invention.

<HP10795> (SEQ ID NOS: 130, 140, and 150)

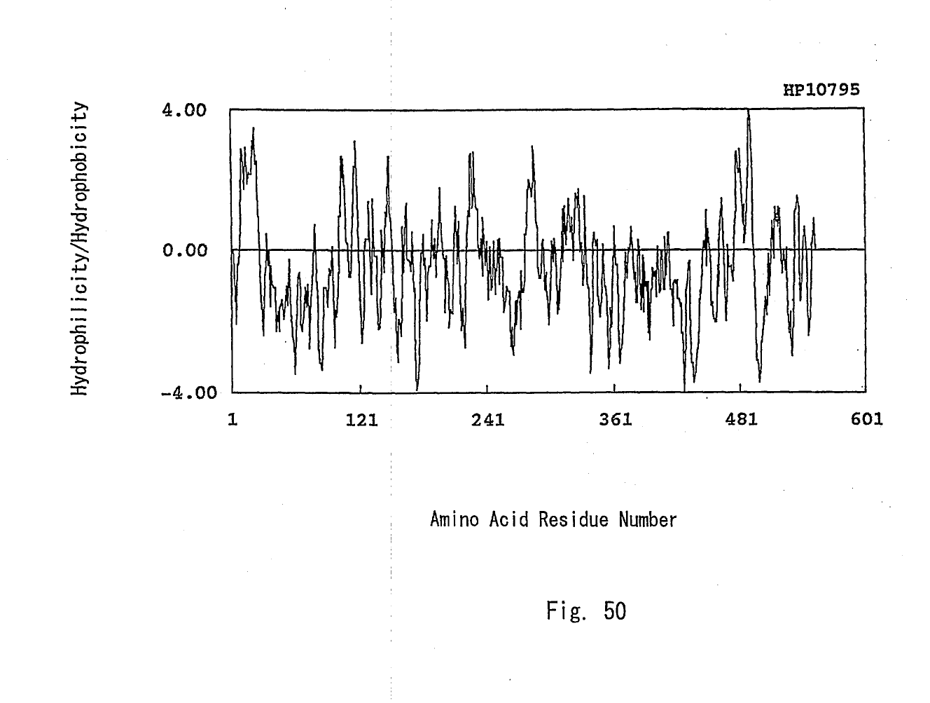

Determination of the whole base sequence of the cDNA insert of clone HP10795 obtained from cDNA library of human kidney revealed the structure consisting of a 356-bp 5′-untranslated region, a 1659-bp ORF, and a 420-bp 3′-untranslated region. The ORF encodes a protein consisting of 552 amino acid residues and there existed one transmembrane domain at the N-terminus. FIG. 50 depicts the hydrophobicity/hydrophilicity profile, obtained by the Kyte-Doolittle method, of the present protein. In vitro translation resulted in formation of a translation product of 65 kDa that was almost identical with the molecular weight of 64,280 predicted from the ORF.

The search of the protein database using the amino acid sequence of the present protein revealed that the protein was similar to human UDP-N-acetyl-α-D-galactosamine:polypeptide N-acetylgalactosaminyltransferase 2 (Accession No. NP_{—}004472). Table 28 shows the comparison between amino acid sequences of the human protein of the present invention (HP) and human UDP-N-acetyl-α-D-galactosamine:polypeptide N-acetylgalactosaminyltransferase 2 (GA). Therein, the marks of -, *, and . represent a gap, an amino acid residue identical with that of the protein of the present invention, and an amino acid residue similar to that of the protein of the present invention, respectively. The both proteins shared a homology of 49.9% in the entire region other than the N-terminal region.

TABLE 28

| | |
|---|---|
| HP | MRRLTRRLVLPVFGVLWITVLLFFWVTKRKLEVPT |
| | ..*..*. .. |
| GA | MRRRSRMLLCFAFLWVLGIAYYMYSGGGSALAGGAGGGAGRKEDWNEIDPIKKKDLHHSN |
| HP | GPEVQTPKPSDADWDDLWDQFDERRYLNAKKWRVGDDPYKLYAFNQRESERISSNRAIPD |
| | * * . .. . . . * .*.. *..... * *.*** *** **.... .***** |
| GA | GEEKAQSMETLPPGKVRWPDFNQEAYVGGTMVRSGQDPYARNKFNQVESDKLRMDRAIPD |
| HP | TRHLRCTLLVYCTDLPPTSIIITFHNEARSTLLRTIRSVLNRTPTHLIREIILVDDPSND |
| | *** .* . .***.**..*********.****. ***...*.***.*******.*** |
| GA | TRHDQCQRKQWRVDLPATSVVITFHNEARSALLRTVVSVLKKSPPHLIKEIILVDDYSND |
| HP | PDDCKQLIKLPKVKCLRNNERQGLVRSRIRGADIAQGTTLTFLDSHCEVNRDWLQPLLHR |
| | *.* * *. **. ***. *.**.***.**** **...********* * .**.***.* |
| GA | PEDGALLGKIEKVRVLRNDRREGLMRSRVRGADAAQAKVLTFLDSHCECNEHWLEPLLER |
| HP | VKEDYTRVVCPVIDIINLDTFTYIESASELRGGFDWSLHFQWEQLSPEQ-KARRLDPTEP |
| | * ** ****.*.**.**.*.* *......*.*****.* *.*. ..*** ..*. .*..* |
| GA | VAEDRTRVVSPIIDVINMDNFQYVGASADLKGGFDWNLVFKWDYMTPEQRRSRQGNPVAP |
| HP | IRTPIIAGGLFVIDKAWFDYLGKYDMDMDIWGGENFEISFRVWMCGGSLEIVPCSRVGHV |
| | *.**.*******.** .*. ****** **.*****.******* *******.******** |
| GA | IKTPMIAGGLFVMDKFYFEELGKYDMMMDVWGGENLEISFRVWQCGGSLEIIPCSRVGHV |
| HP | FRKKHPYVFPDGNANTYIKNTKRTAEVWMDEYKQYYYAARPFALERPFGNVESRIDLRKN |
| | ***.***.**.*..... .**.*.*********..**** * * . *.**..***.***. |
| GA | FRKQHPYTFPGGSGTVFARNTRRAAEVWMDEYKNFYYAAVPSARNVPYGNIQSRLELRKK |
| HP | LRCQSFKWYLENIYPELSIPKESSIQKGNIRQRQKCLESQRQNNQETPNLKLSPCAKVKG |
| | *.*..*******.****..*.. .*. *...* .**.. . .... . . * |
| GA | LSCKPFKWYLENVYPELRVPDHQDIAFGALQQGTNCLDTLGHFADGVVG--VYEC----H |
| HP | EDAKSQVWAFTYTQQILQEELCLSVITLFPGAPVVLVLCKNGDDRQQWTK--TGSHIEHI |
| | ... .* **.* .. . . .***.*.. **. . * *...*.**.*.. ..*.. *. |
| GA | NAGGNQEWALTKEKSVKHMDLCLTVVDRAPGSLIKLQGCRENDSRQKWEQIEGNSKLRHV |
| HP | ASHLCLDTDMFGDGTENGKEIVVNPCESSLMSQHWDMVSS |
| | .*.****. *... .. *. *...* **.* |
| GA | GSNLCLDS---R--TAKSGGLSVEVCGPAL-SQQWKFTLNLQQ |

The search of the GenBank using the base sequences of the present cDNA has revealed the registration of sequences that shared a homology of 90% or more (for example, Accession No. AA160076) among ESTs. However, since they are partial sequences, it can not be judged whether or not they encode the same protein as the protein of the present invention.

Industrial Applicability

The present invention provides human proteins having hydrophobic domains, DNAs encoding these proteins, expression vectors for these DNAs and eukaryotic cells expressing these DNAs. Since all of the proteins of the present invention are secreted or exist in the cell membrane, they are considered to be proteins controlling the proliferation and/or the differentiation of the cells. Accordingly, the proteins of the present invention can be employed as pharmaceuticals such as carcinostatic agents which act to control the proliferation and/or the differentiation of the cells, or as antigens for preparing antibodies against these proteins. The DNAs of the present invention can be utilized as probes for the genetic diagnosis and gene sources for the gene therapy. Furthermore, the DNAs can be utilized for expressing these proteins in large quantities. Cells into which these genes are introduced to express these proteins can be utilized for detection of the corresponding receptors or ligands, screening of novel small molecule pharmaceuticals and the like. The antibody of the present invention can be utilized for the detection, quantification, purification and the like of the protein of the present invention.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information.

Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologs of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide, as determined by those of skill in the art. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encode proteins which are identical, homologous, or related to that encoded by the polynucleotides.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 29

| | Poly- | Hybrid | Hybridization | Wash |
|---|---|---|---|---|
| Stringency | nucleotide | Length | Temperature | Temperature |
| Condition | Hybrid | (bp)^{‡} | and Buffer^{†} | and Buffer^{†} |
| A | DNA:DNA | ≧50 | 65° C.; 1 × SSC | 65° C., |
| | | | -or- 42° C.; | 0.3 × SSC |
| | | | 1 × SSC, 50% | |
| | | | formamide | |
| B | DNA:DNA | <50 | T_{B}*; 1 × SSC | T_{B}*; 1 × SSC |
| C | DNA:RNA | ≧50 | 67° C.; 1 × SSC | 67° C.; |
| | | | -or- 45° C.; | 0.3 × SSC |
| | | | 1 × SSC, 50% | |
| | | | formamide | |
| D | DNA:RNA | <50 | T_{D}*; 1 × SSC | T_{D}*; 1 × SSC |
| E | RNA:RNA | ≧50 | 70° C.; 1 × SSC | 70° C.; |
| | | | -or- 50° C.; | 0.3 × SSC |
| | | | 1 × SSC, 50% | |
| | | | formamide | |
| F | RNA:RNA | <50 | T_{F}*; 1 × SSC | T_{F}*; 1 × SSC |
| G | DNA:DNA | ≧50 | 65° C.; 4 × SSC | 65° C.; 1 × SSC |
| | | | -or- 42° C.; | |
| | | | 4 × SSC, 50% | |
| | | | formamide | |
| H | DNA:DNA | <50 | T_{H}*; 4 × SSC | T_{H}*; 4 × SSC |
| I | DNA:RNA | ≧50 | 67° C.; 4 × SSC | 67° C.; 1 × SSC |
| | | | -or- 45° C.; | |
| | | | 4 × SSC, 50% | |
| | | | formamide | |
| J | DNA:RNA | <50 | T_{J}*; 4 × SSC | T_{J}*; 4 × SSC |
| K | RNA:RNA | ≧50 | 70° C.; 4 × SSC | 67° C.; 1 × SSC |
| | | | -or- 50° C.; | |
| | | | 4 × SSC, 50% | |
| | | | formamide | |
| L | RNA:RNA | <50 | T_{L}*; 2 × SSC | T_{L}*; 2 × SSC |
| M | DNA:DNA | ≧50 | 50° C.; 4 × SSC | 50° C.; 2 × SSC |
| | | | -or- 40° C.; | |
| | | | 6 × SSC, 50% | |
| | | | formamide | |
| N | DNA:DNA | <50 | T_{N}*; 6 × SSC | T_{N}*; 6 × SSC |
| O | DNA:RNA | ≧50 | 55° C.; 4 × SSC | 55° C.; 2 × SSC |
| | | | -or- 42° C.; | |
| | | | 6 × SSC, 50% | |
| | | | formamide | |
| P | DNA:RNA | <50 | T_{P}*; 6 × SSC | T_{P}*; 6 × SSC |
| Q | RNA:RNA | ≧50 | 60° C.; 4 × SSC | 60° C.; 2 × SSC |
| | | | -or- 45° C.; | |
| | | | 6 × SSC, 50% | |
| | | | formamide | |
| R | RNA:RNA | <50 | T_{R}*; 4 × SSC | T_{R}*; 4 × SSC |

≠: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.

†: SSPE (1×SSPE is 0.15M NaCl, 10 mM NaH₂PO₄, and 1.25 mM EDTA, pH7.4) can be substituted for SSC (1×SSC is 0.15M NaCl and 15 mM 'sodium citrate) in the hybridization and wash buffers; washes are performed for 15 minutes after hybridization is complete.

*T_{B}−T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(°C.)=2(#of A+T bases)+4(# of G+C bases). For hybrids between 18 and 49 base pairs in length, Tₘ(°C.)=81.5+16.6(log₁₀[Na⁺])+0.41 (% G+C)−(600/N), where N is the number of bases in the hybrid, and [Na⁺] is the concentration of sodium ions in the hybridization buffer ([Na⁺] for 1×SSC=0.165M).

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

1. A protein comprising any one of amino acid sequences selected from the group consisting of SEQ ID NOS: 1 to 10, 31 to 40, 61 to 70, 91 to 100 and 121 to 130.

2. An isolated DNA encoding the protein according to claim 1.

3. An isolated cDNA comprising any one of base sequences selected from the group consisting of SEQ ID NOS: 11 to 20, 41 to 50, 71 to 80, 101 to 110 and 131 to 140.

4. The cDNA according to claim 3 consisting of any one of base sequences selected from the group consisting of SEQ ID NOS: 21 to 30, 51 to 60, 81 to 90, 111 to 120 and 141 to 150.

5. An expression vector that is capable of expressing the DNA according to any one of claim 2 to claim 4 by in vitro translation or in eukaryotic cells.

6. A transformed eukaryotic cell that is capable of expressing the DNA according to any one of claim 2 to claim 4 and of producing the protein according to claim 1.

7. An antibody directed to the protein according to claim 1.
